(19) United States
(12) Patent Application Publication
(10) Pub. No.: US 2026/0274922 A1
(43) Pub. Date: Sep. 17, 2026

(54) SYNTHETIC ESTROGEN RECEPTOR (ER)-DERIVED PEPTIDE

(71) Applicant: THE TEXAS A&M UNIVERSITY SYSTEM, College Station, TX (US)

(72) Inventors: Wanbao YANG, College Station, TX (US); Shaodong GUO, College Station, TX (US)

(21) Appl. No.: 19/470,254

(22) PCT Filed: Mar. 29, 2024
(86) PCT No.: PCT/US2024/022205

§ 371 Date: Sep. 29, 2025

Related U.S. Application Data
(60) Provisional application No. 63/493,456, filed on Mar. 31, 2023.

(51) Int. Cl. C07K 14/72; A61K 38/00; A61K 49/00; A61P 3/10

(52) U.S. Cl. CPC C07K 14/721; A61K 49/0043; A61K 49/0056; A61P 3/10; A61K 38/00; C07K 2319/33

(57) ABSTRACT

The present disclosure relates to a synthetic estrogen receptor alpha peptide, compositions, and methods thereof for the use of enhancing insulin sensitivity and treating and/or preventing diabetes.

### CROSS-REFERENCE TO RELATED APPLICATIONS

This PCT application claims priority to, and the benefit of, U.S. Provisional Patent Application No. 63/493,456, filed Mar. 31, 2023, which is incorporated by reference herein in its entirety.

### STATEMENT REGARDING FEDERALLY SPONSORED RESEARCH

This invention was made with Government Support under Grant No. R01 DK120968 awarded by the National Institutes of Health. The Government has certain rights in the invention.

### REFERENCE TO SEQUENCE LISTING

The sequence listing submitted on Mar. 29, 2024, as an XML file entitled “11164-017WO1 ST26” created on Mar. 25, 2024, and having a file size of 27,701 bytes is hereby incorporated by reference pursuant to 37 C.F.R. § 1.52 (e)(5).

### FIELD

The present disclosure relates to a synthetic estrogen receptor alpha peptide, compositions, and methods thereof for the use of enhancing insulin sensitivity and treating and/or preventing diabetes.

### BACKGROUND

Commonly diagnosed in over 400 million people worldwide, diabetes remains undiagnosed in almost 200 million people. The majority of these diagnosed and undiagnosed cases are type II diabetes cases presenting with insulin resistance and relative insulin deficiency. Current treatments of type II diabetes require diligent observation and management of patient medications, exercise, and nutrition. In addition, current antidiabetic medications have been developed to target hepatic glucose production (HGP), insulin secretion, and insulin sensitivity. However, in many instances despite proper care and management, patients remain insulin resistant and require further intervention to properly regulate blood glucose levels.

Given limitations of current antidiabetic medications and the complexities of diabetes, there is need to address the aforementioned problems mentioned above by developing therapeutic targets of diabetes that will overcome insulin resistance and improve insulin sensitivity. The peptides, compositions, and methods disclosed herein address these and other needs.

### SUMMARY

The present disclosure provides a synthetic peptide derived from an estrogen receptor alpha (ERα) protein.

In one aspect, disclosed herein is a synthetic peptide comprising: a peptide from an activation function 1 (AF1) domain of an estrogen receptor alpha (ERα) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids.

In some embodiments, the at least 30 amino acids from the AF1 domain of the ERα protein are selected from the first 60 amino acids of the ERα protein.

In some embodiments, the synthetic peptide comprises 90% sequence identity to SEQ ID NO: 1. In some embodiments, the synthetic peptide comprises 99% sequence identity to SEQ ID NO: 1. In some embodiments, the synthetic peptide comprises SEQ ID NO: 1.

In some embodiments, the AF1 domain comprises SEQ ID NO: 2, or a variant or fragment thereof. In some embodiments, the cell penetrating peptide comprises SEQ ID NO: 3, or a variant or fragment thereof.

In some embodiments, the cell penetrating peptide comprises a transactivator of transcription (TAT) peptide.

In some embodiments, the synthetic peptide is further linked to a light emitting molecule. In some embodiments, the light emitting molecule comprises a fluorescent molecule. In some embodiments, the light emitting molecule comprises a fluorescein molecule, or a derivative thereof.

The present disclosure also provides a nucleic acid encoding the synthetic peptide derived from an estrogen receptor alpha (ERα) protein.

In one aspect, disclosed herein is a nucleic acid encoding the synthetic peptide of any preceding aspect.

The present disclosure also provides a pharmaceutical composition comprising the synthetic peptide derived from an estrogen receptor alpha (ERα) protein.

In one aspect, disclosed herein is a pharmaceutical composition for enhancing insulin sensitivity comprising a synthetic peptide comprising a peptide from an activation function 1 (AF1) domain of an estrogen receptor alpha (ER) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids, and a pharmaceutically acceptable carrier selected from an excipient, a diluent, a salt, a buffer, a stabilizer, a solubilizer, a lipid, an emulsion, and nanoparticle.

The present disclosure also provides methods using the synthetic peptide derived from an estrogen receptor alpha (ERα) protein to treat/prevent diabetes.

In one aspect, disclosed herein is a method of treating or preventing *diabetes in a subject in need thereof, the method comprising administering a therapeutically effective amount of a synthetic peptide comprising: a peptide from an activation function 1 (AF1) domain of an estrogen receptor alpha (ERα) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids.

In some embodiments, the synthetic peptide comprises 90% sequence identity to SEQ ID NO: 1. In some embodiments, the synthetic peptide comprises 99% sequence identity to SEQ ID NO: 1. In some embodiments, the synthetic peptide comprises SEQ ID NO: 1.

In some embodiments, the synthetic peptide interacts with an insulin receptor substrate (IRS) to activate insulin signaling in the subject. In some embodiments, the IRS comprises an insulin receptor substrate 1 (IRS1).

In some embodiments, the synthetic peptide enhances insulin sensitivity in the subject.

In some embodiments, the AF1 domain comprises SEQ ID NO: 2, or a variant or fragment thereof.

In some embodiments, the cell penetrating peptide comprises SEQ ID NO: 3, or a variant or fragment thereof.

In some embodiments, the ER is an estrogen receptor α (ERα). In some embodiments, the cell penetrating peptide comprises a transactivator of transcription (TAT) peptide. In some embodiments, the cell penetrating peptide directs the synthetic peptide into a cell. In some embodiments, the cell is a liver cell, a muscle cell, or an adipose cell.

In some embodiments, the synthetic peptide is further linked to a light emitting molecule. In some embodiments, the light emitting label comprises a fluorescent label. In some embodiments, the light emitting label comprises a fluorescein label, or a derivative thereof.

In some embodiments, the type of diabetes is Type II diabetes.

The present disclosure also provides methods using the synthetic peptide derived from an estrogen receptor alpha (ERα) to enhance insulin sensitivity.

In one aspect, disclosed herein is a method of enhancing insulin sensitivity in a subject, the method comprising administering a therapeutically effective amount of a synthetic peptide comprising: a peptide from an activation function 1 (AF1) domain of an estrogen receptor alpha (ERα) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids.

In some embodiments, the synthetic peptide comprises 90% sequence identity to SEQ ID NO: 1. In some embodiments, the synthetic peptide comprises 99% sequence identity to SEQ ID NO: 1. In some embodiments, the synthetic peptide comprises SEQ ID NO: 1.

In some embodiments, the synthetic peptide interacts with an insulin receptor substrate (IRS) to activate insulin signaling and increase insulin sensitivity in the subject. In some embodiments, the IRS comprises an insulin receptor substrate 1 (IRS1).

In some embodiments, the AF1 domain comprises SEQ ID NO: 2, or a variant or fragment thereof.

In some embodiments, the cell penetrating peptide comprises SEQ ID NO: 3, or a variant or fragment thereof.

In some embodiments, the ER is an estrogen receptor α (ERα) protein.

In some embodiments, the cell penetrating peptide comprises a transactivator of transcription (TAT) peptide. In some embodiments, the cell penetrating peptide is located at the amino terminus (N-terminal) end of the synthetic peptide. In some embodiments, the cell penetrating peptide directs the synthetic peptide into a cell. In some embodiments, the cell is a liver cell, a muscle cell, or an adipose cell.

In some embodiments, the synthetic peptide is further linked to a light emitting molecule. In some embodiments, the light emitting molecule is a fluorescent molecule. In some embodiments, the light emitting molecule is a fluorescein molecule, or a derivative thereof.

In some embodiments, the synthetic peptide treats or prevents a type of diabetes in the subject. In some embodiments, the type of diabetes is Type II diabetes.

### BRIEF DESCRIPTION OF FIGURES

The accompanying figures, which are incorporated in and constitute a part of this specification, illustrate several aspects described below.

FIGS. 1A, 1B, 1C, 1D, 1E, 1F, 1G, 1H, 1I, 1J, 1K, and 1L show that the hepatic ERα knockout impairs glucose tolerance and insulin sensitivity in both male and female mice. FIG. 1A shows IRS1, IRS2, and ERα protein levels in the livers of random-feeding WT and db/db mice, n=4 mice/group. FIG. 1B shows the mRNA expression levels of ERα in the livers of random feeding WT and db/db mice, n=4 mice/group. FIG. 1C shows the mRNA expression levels of ERα in the livers of humans with diabetes, n=4-5/group. FIG. 1D shows the Pearson correlation coefficient between the Fasting blood glucose/HbA1C/HOMA-IR/Fasting insulin and the mRNA expression levels of ERα in the livers of humans, n=17-18. FIG. 1E shows the random feeding and 5 h fasting blood glucose in ERα^{F/F} and ERα^{LivKO} male mice, n=6 (ERα^{LivKO}) and 9 (ERα^{F/F}) mice/group. FIG. 1F shows the glucose tolerance test in ERα^{F/F} and ERα^{LivKO} male mice, n=8 (ERα^{LivKO}) and 10 (ERα^{F/F}) mice/group. FIG. 1G shows the insulin tolerance test in ERα^{F/F} and ERα^{LivKO} male mice, n=6 (ERα^{LivKO}) and 7 (ERα^{F/F}) mice/group. FIG. 1H shows that the insulin signaling was detected in livers from ERα^{F/F} and ERα^{LivKO} male mice injected with 2 U insulin for 5 min. Representative results were shown. FIG. 1I shows the random feeding and 5 h fasting blood glucose in ERα^{F/F} and ERα^{LivKO} female mice, n=7 mice/group. j Glucose tolerance test in ERα^{F/F} and ERα^{LivKO} female mice, n=7 mice/group. FIG. 1K shows the insulin tolerance test in ERα^{F/F} and ERα^{LivKO} female mice, n=7 mice/group. FIG. 1L shows the insulin signaling was detected in livers from ERα^{F/F} and ERα^{LivKO} female mice injected with 2 U insulin for 5 min. Representative results were shown. Data are presented as mean±SEM. * p<0.05, ** p<0.01, *** p<0.001, **** p<0.0001, Student's t test (FIGS. 1A-1B, 1E-1G, and 1O-1K) or One-way ANOVA (FIG. 1C).

FIGS. 2A, 2B, 2C, 2D, 2E, 2F, 2G, 2H, 2I, and 2J show the deletion of hepatic ERα impairs glucose tolerance and insulin sensitivity in both DIO male and female mice. FIG. 2A shows the body weight of ERα^{F/F} and ERα^{LivKO} male mice treated with HFD, n=6 (ERα^{F/F}) and 9 (ERα^{LivKO}) mice/group. FIG. 2B shows the body composition of ERα^{F/F} and ERα^{LivKO} male mice treated with HFD, n=6 (ERα^{F/F}) and 9 (ERα^{LivKO}) mice/group. FIG. 2C shows the random feeding and 16 h fasting blood glucose in ERα^{F/F} and ERα^{LivKO} male mice treated with HFD, n=6 (ERα^{F/F}) and 8 (ERα^{LivKO}) mice/group. FIG. 2D shows the glucose tolerance tests in ERα^{F/F} and ERα^{LivKO} male mice treated with HFD, n=6 (ERα^{F/F}) and 8 (ERα^{LivKO}) mice/group. FIG. 2E shows the insulin tolerance tests in ERα^{F/F} and ERα^{LivKO} male mice treated with HFD, n=6 (ERα^{F/F}) and 8 (ERα^{LivKO}) mice/group. FIG. 2F shows the body weight of ERα^{F/F} and ERα^{LivKO} female mice treated with HFD, n=7 (ERα^{LivKO}) and 10 (ERα^{F/F}) mice/group. FIG. 2G shows the body composition of ERF/F and ERα^{LivKO} female mice treated with HFD, n=7 (ERα^{LivKO}) and 10 (ERα^{F/F}) mice/group. FIG. 2H shows the random feeding and 16 h fasting blood glucose in ERα^{F/F} and ERα^{LivKO} female mice treated with HFD, n=7 (ERα^{LivKO}) and 10 (ERα^{F/F}) mice/group. FIG. 2I shows the glucose tolerance tests in ERα^{F/F} and ERα^{LivKO} female mice treated with HFD, n=7 (ERα^{LivKO}) and 10 (ERα^{F/F}) mice/group. FIG. 2J shows the insulin tolerance tests in ERα^{F/F} and ERα^{LivKO} female mice treated with HFD, n=7 (ERα^{LivKO}) and 10 (ERα^{F/F}) mice/group. Data are presented as mean±SEM. * p<0.05, ** p<0.01, *** p<0.001, Student's t test.

FIGS. 3A, 3B, 3C, 3D, 3E, 3F, 3G, 3H, 3I, 3J, 3K, 3L, 3M, 3N, 3O, 3P, 3Q, 3R, 3S, and 3T show that the hepatic ERα regulates glucose homeostasis and insulin sensitivity in an IRS1/2-independent manner. FIG. 3A shows the random feeding and 16 h fasting blood glucose levels in control (CNTR), DKO, and TKO male mice under regular chow diet, n=7 (DKO), 9 (TKO), and 11 (control) mice/group. FIGS. 3B and 3C show the glucose tolerance tests in control, DKO, and TKO male mice under regular chow diet, n=7 (DKO) and 11 (control and TKO) mice/group. FIGS. 3D and 3E show the insulin tolerance tests in control, DKO, and TKO male mice under regular chow diet, n=7 (DKO) and 11 (control and TKO) mice/group. FIG. 3F shows the random feeding and 16 h fasting blood glucose levels in control, DKO, and TKO female mice under regular chow diet, n=6 (DKO and TKO) and 8 (control) mice/group. FIGS. 3G and 3H show the glucose tolerance tests in control, DKO, and TKO female mice under regular chow diet, n=7 (DKO and TKO) and 11 (control) mice/group. FIGS. 3I and 3J show the insulin tolerance tests in control, DKO, and TKO female mice under regular chow diet, n=7 (DKO and TKO) and 11 (control) mice/group. FIG. 3K shows the random feeding and 16 h fasting blood glucose levels in control and DKO male/female mice under regular chow diet, n=5 (Male-DKO and Female-CNTR) and 6 (Male-CNTR and Female-DKO) mice/group. FIGS. 3L and 3M show the glucose tolerance tests in control and DKO male/female mice under regular chow diet, n=5 (Male-DKO and Female-CNTR) and 6 (Male-CNTR and Female-DKO) mice/group. FIGS. 3N and 3O show the insulin tolerance tests in control and DKO male/female mice under regular chow diet, n=5 (Male-DKO and Female-CNTR) and 6 (Male-CNTR and Female-DKO) mice/group. FIG. 3P shows the random feeding and 16 h fasting blood glucose levels in control and DKO male/OVX female mic under regular chow diet e, n=5 (Male-DKO, OVX Female-CNTR, and OVX Female-DKO) and 6 (Male-CNTR) mice/group. FIGS. 3Q and 3R show the glucose tolerance tests in control and DKO male/OVX female mice under regular chow diet, n=5 (Male-DKO, OVX Female-CNTR, and OVX Female-DKO) and 6 (Male-CNTR) mice/group. FIGS. 3S and 3T show the insulin tolerance tests in control and DKO male/OVX female mice under regular chow diet, n=5 mice/group. Data are presented as mean±SEM. * p<0.05, ** p<0.01, *** p<0.001, **** p<0.0001, One-way ANOVA (FIGS. 3A-3J) or Two-way ANOVA (FIGS. 3K-3T).

FIGS. 4A, 4B, 4C, 4D, 4E, 4F, 4G, 4H, 4I, 4J, 4K, 4L, and 4M show ERα increases IRS1 protein stability and promotes hepatic insulin sensitivity. FIG. 4A shows the insulin signaling activity in primary hepatocytes from ERα^{F/F} and ERα^{LivKO} male mice, n=3. FIG. 4B shows the effect of ERα gain-of-function on hepatic insulin sensitivity, representative blots were shown. FIG. 4C shows the effect of ERα deletion on insulin-induced IRS1 and p85 interaction, representative blots were shown. FIG. 4D shows the effect of ERα gain-of-function on insulin-induced IRS1 and p85 interaction. Representative blots were shown. FIG. 4E shows the effect of ERα deletion on mRNA expression of IRS1 and IRS2 in primary mouse hepatocytes, n=4-5. FIG. 4F shows the interaction between ERα and IRS1 or IRS2 in HEK293T cells. FIG. 4G shows the effect of ERα on IRS1 ubiquitination in HEK293T cells. FIG. 4H shows the effect of ERα on IRS2 ubiquitination in HEK293T cells. FIG. 4I shows the effect of ERα deletion on IRS1 ubiquitination in primary mouse hepatocytes. FIG. 4J shows the diagram of IRS1 and its truncated domains. FIG. 4K shows the interaction between ERα and IRS1 domains in HEK293T cells. FIG. 4L shows the effect of ERα deletion on IRS1 phosphorylation in primary mouse hepatocytes, n=3. Representative blots were shown. FIG. 4M shows the effect of ERα gain-of-function on palmitate-induced insulin resistance in primary mouse hepatocytes. Data are presented as mean±SEM. * p<0.05, *** p<0.001, **** p<0.0001, Student's t test (FIGS. 4E and 4L), Two-way ANOVA (FIG. 4A).

FIGS. 5A, 5B, 5C, 5D, 5E, 5F, 5G, 5H, and 5I show the ERα 1-280 domain increases IRS1 protein stability and enhances insulin signaling. FIG. 5A shows the diagram of truncated ERα protein domains. FIG. 5B shows the effect of ERα on insulin sensitivity in HepG2 cells, n=3. Representative blots were shown. FIG. 5C shows the effect of ERα 1-280 on insulin sensitivity in HepG2 cells, n=3. Representative blots were shown. FIG. 5D shows the effect of ERα DBD+AF2 on insulin sensitivity in HepG2 cells, n=3. Representative blots were shown. e Interaction between ERα 1-280 and IRS1 protein domains. Representative blots were shown. FIG. 5F shows the effect of ERα 1-280 on IRS1 phosphorylation at S302 in HepG2 cells, n=3. Representative blots were shown. FIG. 5G shows the effect of ERα on IRS1 phosphorylation at S302 in HepG2 cells, n=3. Representative blots were shown. FIG. 5H shows the effect of ERα 1-280 on IRS1 ubiquitination in HEK293T cells. Representative blots were shown. FIG. 5I shows the effect of ERα 1-280 on IRS2 ubiquitination in HEK293T cells. Representative blots were shown. Data are presented as mean±SEM. * p<0.05, ** 0.01, *** p<0.001, **** p<0.0001, Student's t test (FIGS. 5F and 5G), Two-way ANOVA (FIGS. 5B, 5C, and 5D).

FIGS. 6A, 6B, 6C, 6D, 6E, 6F, 6G, 6H, 6I, 6J, and 6K show the ERα-derived AF1 peptide increases hepatic insulin sensitivity through IRS1. FIG. 6A shows the SVM score of interaction between IRS1 and ERα domains. FIG. 6B shows the effect of ERα 1-60 domain and AF1 peptide (34 aa) on insulin sensitivity in HepG2 cells. FIG. 6C shows the effect of ERα 60-280 domain on insulin sensitivity in HepG2 cells. FIG. 6D shows the immunofluorescence staining of AF1 peptide (34 aa) and ERα 1-60 domain in HEK293T cells. FIG. 6E shows the amino acid sequence of FITC labelled AF1 peptide conjugated with TAT. FIG. 6F shows the interaction between AF1 peptide and IRS1 protein domains. Representative results were shown. FIG. 6G shows the effect of AF1 peptide on IRS1 phosphorylation at S302 in primary mouse hepatocytes, n=3. Representative blots were shown. FIG. 6H shows the effect of AF1 peptide on IRS1 ubiquitination in HEK293T cells. Representative blots were shown. FIG. 6I shows the effect of AF1 peptide on insulin-induced suppression of HGP in primary hepatocytes upon glucagon treatment, n=4. Representative results were shown. FIG. 6J shows the effect of AF1 peptide on insulin sensitivity in primary mouse hepatocytes, n=3. Representative blots were shown. FIG. 6K shows the effect of AF1 peptide on insulin sensitivity in control and ERα deficient primary mouse hepatocytes, n=3. Representative blots were shown. Data are presented as mean±SEM. * p<0.05, *** p<0.001, **** p<0.0001, Student's t test (FIGS. 6I and 6G), One-way ANOVA (FIGS. 6B, 6I, and 6K), Two-way ANOVA (FIGS. 6C and 6J).

FIGS. 7A, 7B, 7C, 7D, 7E, 7F, 7G, 7H, 7I, 7J, and 7K show that the AF1 peptide improves glucose tolerance and insulin sensitivity in diabetic mice. FIG. 7A shows the body weight of db/db mice treated with control and AF1 peptide for 5 weeks, n=5 (AF1 peptide) and 6 (control) mice/group. FIG. 7B shows the random feeding and 16 h fasting blood glucose in db/db mice treated with control and AF1 peptide for 5 weeks, n=6 mice/group. FIG. 7C shows the glucose tolerance test in db/db mice treated with control and AF1 peptide for 5 weeks, n=6 mice/group. FIG. 7D shows the insulin tolerance test in db/db mice treated with control and AF1 peptide for 5 weeks, n=6 mice/group. FIG. 7E shows the H&E staining of livers from db/db mice treated with control and AF1 peptide. Scale: 200 μm. Liver fat content was calculated, n=5 mice/group. Representative results were shown. FIG. 7F shows the liver insulin sensitivity in db/db mice treated with control and AF1 peptide, n=5 mice/group. FIG. 7G shows the phosphorylation of AKT in epididymal fat and skeleton muscle from db/db mice treated with control and AF1 peptide, n=4 mice/group. FIG. 7H shows the volcano plots of differentially expressed genes (DEGs) in livers from db/db mice treated with control and AF1 peptide. FIG. 7I shows the KEGG pathway analysis of DEGs in livers from db/db mice treated with control and AF1 peptide. FIG. 7J shows the heatmap of representative DEGs in livers from db/db mice treated with control and AF1 peptide. FIG. 7K shows the liver triglyceride, serum triglyceride, AST, cholesterol, LDL, and NEFA levels in db/db mice treated with control and AF1 peptide, n=4-6 mice/group. Data are presented as mean±SEM. * p<0.05, ** p<0.01, Student's t test.

FIGS. 8A and 8B show the hyperinsulinemia decreases ERα mRNA expression in mouse primary hepatocytes. FIG. 8A shows the fasting insulin levels in healthy and diabetic patients, n=3-5. FIG. 8B shows the mRNA expression levels of ERα in mouse primary hepatocytes treated with 100 nM and 200 nM insulin for 12 hours, n=3. Data are presented as mean±SEM. * p<0.05, One-way ANOVA.

FIGS. 9A, 9B, 9C, 9D, 9E, 9F, 9G, 9H, 9I, 9J, 9K, 9L, 9M, 9N, and 9O show the effect of hepatic ERβ on glucose homeostasis in show diet feeding mice. FIG. 9A shows the quantification of FIG. 1H, n=3-4 mice/group. FIG. 9B shows the quantification of FIG. 1L, n=3-4 mice/group. FIG. 9C shows the serum estradiol levels in intact and OVX female mice, n=5 mice/group. FIG. 9D shows the random feeding and 5 h fasting blood glucose in ERαF/F and ERαLivKO OVX female mice, n=6 mice/group. FIG. 9E shows the glucose tolerance tests in ERαF/F and ERαLivKO OVX female mice, n=6 mice/group. FIG. 9F shows the insulin tolerance tests in ERαF/F and ERαLivKO OVX female mice, n=6 mice/group. FIGS. 9G and 9H show the insulin signaling was detected in the livers of ERαF/F and ERαLivKO OVX female mice injected with 2 U insulin for 5 min, n=3 mice/group. Representative results were shown. FIG. 9I shows the random feeding and 16 h fasting blood glucose in ERβF/F and ERβLivKO male mice, n=6 (ERβLivKO) and 8 (ERβF/F) mice/group. FIG. 9J shows the glucose tolerance tests in ERβF/F and ERβLivKO male mice, n=7 (ERβLivKO) and 8 (ERβF/F) mice/group. FIG. 9K shows the insulin tolerance tests in ERβF/F and ERβLivKO male mice, n=7 (ERβLivKO) and 8 (ERβF/F) mice/group. FIG. 9L shows the random feeding and 16 h fasting blood glucose in ERβF/F and ERβLivKO female mice, n=7 (ERβF/F) and 11 (ERβLivKO) mice/group. FIGS. 9M and 9N show the glucose tolerance tests in ERβF/F and ERβLivKO female mice, n=7 (ERβF/F) and 11 (ERβLivKO) mice/group. FIG. 9O shows the insulin tolerance tests in ERβF/F and ERβLivKO female mice, n=7 (ERβF/F) and 11 (ERβLivKO) mice/group. Data are presented as mean±SEM. * p<0.05, ** p<0.01, *** p<0.001, **** p<0.0001, Student's t test (FIGS. 9D, 9F, and 9I-9O), One-way ANOVA (FIG. 9C), or Two-way ANOVA (FIGS. 9A, 9B, and 9H).

FIGS. 10A, 10B, 10C, 10D, 10E, 10F, 10G, 10H, 10I, 10J, 10K, 10L, and 10M show the hepatic insulin sensitivity in control, DKO, and TKO male/female mice. FIG. 10A shows the effect of ERα on hepatic insulin sensitivity in control and DKO male mice injected with 2 U insulin for 5 min, n=3 mice/group. Representative blots were shown. FIG. 10B shows the mRNA expression levels of gluconeogenic genes in the livers of control and DKO male mice under random feeding, n=5 (control and TKO) and 6 (DKO) mice/group. FIG. 10C shows the effect of ERα on hepatic insulin sensitivity in control and DKO female mice injected with 2 U insulin for 5 min, n=3 mice/group. Representative blots were shown. FIG. 10D shows the mRNA expression levels of gluconeogenic genes in the livers of control and DKO female mice under random feeding, n=5 mice/group. FIG. 10E shows the hepatic insulin sensitivity in control and DKO male/female mice injected with 2 U insulin for 5 min, n=3 mice/group. Representative blots were shown. FIG. 10F shows the mRNA expression levels of gluconeogenic genes in the livers of control and DKO male/female mice under random feeding, n=5 mice/group. FIG. 10G shows the hepatic insulin sensitivity in control and DKO male/OVX female mice injected with 2 U insulin for 5 min, n=3 mice/group. Representative blots were shown. FIG. 10H shows the mRNA expression levels of gluconeogenic genes in the livers of control, DKO male, DKO OVX female, and DKO OVX-E2 supplemented mice under random feeding, n=5 mice/group. FIGS. 10I and 10J show the glucose tolerance tests in OVX control, OVX female, and OVX female-E2 supplemented mice, n=5 (OVX female and OVX female-E2) and 6 (OVX control) mice/group. FIGS. 10K and 10L show the insulin tolerance tests in OVX control, OVX female, and OVX female-E2 supplemented mice, n=5 (OVX female and OVX female-E2) and 6 (OVX control) mice/group. FIG. 10M shows the hepatic insulin sensitivity in OVX control, OVX female, and OVX female-E2 supplemented mice injected with 2 U insulin for 5 min, n=3 mice/group. Representative blots were shown. Data are presented as mean±SEM. * p<0.05, ** p<0.01, *** p<0.001, **** p<0.0001, One-way ANOVA (FIGS. 10A-10D and 10I-10M), Two-way ANOVA (FIGS. 10E-10H).

FIGS. 11A and 11B show the ERα interacts with IRS1 and regulates IRS1 protein stability. FIG. 11A shows the IRS1 protein levels in control and ERα deficient mouse primary hepatocytes treated with 10 μM MG132 for 3 hours, n=3. Representative blots are shown. FIG. 11B shows the interaction between ERα and IRS1 in HEK293 cells treated with 100 nM E₂ for 1 hour. HA-ERα and Flag-IRS1 plasmids were transfected into HEK293 cells for 30 hours, followed by 100 nM E₂ treatment for 1 hour. Co-immunoprecipitation was performed. Data are presented as mean±SEM. ** p<0.01, *** p<0.001, Two-way ANOVA.

FIGS. 12A, 12B, 12C, 12D, 12E, 12F, 12G, 12H, 12I, 12J, 12K, 12L, 12M, 12N, 12O, 12P, 12Q, 12R, 12S, and 12T show that ERα and ERα 1-280 improves glucose tolerance and insulin sensitivity in mice. FIG. 12A shows the random feeding blood glucose in male mice infected with Ad-GFP and Ad-ERα, n=5 mice/group. FIGS. 12B and 12C show the glucose tolerance tests in male mice infected with Ad-GFP and Ad-ERα, n=5 mice/group. FIGS. 12D and 12E shows the insulin tolerance tests in male mice infected with Ad-GFP and Ad-ERα, n=5 mice/group. FIGS. 12F and 12G show the effect of ERα gain-of-function on hepatic insulin sensitivity in male mice treated with 2 U insulin for 5 min, n=3 mice/group. Representative blots were shown. FIG. 12H shows the random feeding and 16 h fasting blood glucose in male mice infected with Ad-GFP and Ad-ERα 1-280, n=5 (Ad-ERα 1-280) and 7 (Ad-GFP) mice/group. FIGS. 12I and 12J shows the glucose tolerance tests in male mice infected with Ad-GFP and Ad-ERα 1-280, n=5 (Ad-ERα 1-280) and 7 (Ad-GFP) mice/group. FIGS. 12K and 12L show the insulin tolerance tests in male mice infected with Ad-GFP and Ad-ERα 1-280, n=5 (Ad-ERα 1-280) and 7 (Ad-GFP) mice/group. FIGS. 12M and 12N show the effect of ERα 1-280 gain-of-function on hepatic insulin sensitivity in male mice treated with 2 U insulin for 5 min, n=3 mice/group. Representative blots were shown. FIG. 12O show the random feeding and 16 h fasting blood glucose in female mice infected with Ad-GFP and Ad-ERα 1-280, n=5 (Ad-GFP) and 6 (Ad-ERα 1-280) mice/group. FIGS. 12P and 12Q shows the glucose tolerance tests in female mice infected with Ad-GFP and Ad-ERα 1-280, n=5 (Ad-GFP) and 6 (Ad-ERα 1-280) mice/group. FIG. 12R shows the insulin tolerance tests in female mice infected with Ad-GFP and Ad-ERα 1-280, n=5 (Ad-GFP) and 6 (Ad-ERα 1-280) mice/group. FIGS. 12S and 12T show the effect of ERα 1-280 gain-of-function on hepatic insulin sensitivity in female mice treated with 2 U insulin for 5 min, n=3 mice/group. Representative blots were shown. Data are presented as mean±SEM. * p<0.05, ** p<0.01, *** p<0.001, **** p<0.0001, Students' t test (FIGS. 12A-12E, 12H-12L, and 12O-12R), Two-way ANOVA (FIGS. 12F-12G, 12M-12N, and 12S-12T).

FIGS. 13A and 13B show the optimization of AF1 peptide. FIG. 13A shows the SVM interaction score between ERα domains and IRS1. FIG. 1B shows the AF1 peptide sequence similarity among different species.

FIGS. 14A, 14B, 14C, 14D, 14E, 14F, 14G, 14H, 14I, 14J, 14K, 14L, 14M, and 14N show that the AF1 peptide improves glucose homeostasis in HFD-induced obesity mice. FIG. 14A shows the body weight, serum insulin, ALP, ALT, and HDL levels in db/db mice treated with control and AF1 peptide, n=5-6 mice/group. FIG. 14B shows the Oil Red O staining in the livers of db/db mice treated with control and AF1 peptide. Representative images were shown. FIG. 14C shows the body weight of HFD-induced obesity mice treated with control and AF1 peptide, n=5 (AF1 peptide) and 6 (control) mice/group. FIG. 14D shows the random feeding and 16 h fasting blood glucose in HFD-induced obesity mice treated with control and AF1 peptide, n=5 (AF1 peptide) and 6 (control) mice/group. FIGS. 14E and 14F show the glucose tolerance tests in HFD-induced obesity mice treated with control and AF1 peptide, n=5 (AF1 peptide) and 6 (control) mice/group. FIGS. 14G and 14H show the glucose tolerance tests in HFD-induced obesity mice treated with control and AF1 peptide, n=5 (AF1 peptide) and 6 (control) mice/group. FIG. 14I shows the H&E staining of livers from HFD-induced obesity mice treated with control and AF1 peptide. Scale: 200 μm. Representative images were shown. FIGS. 14J and 14K show the effect of AF1 peptide on hepatic insulin sensitivity in HFD-induced obesity mice, n=3 mice/group. Representative blots were shown. FIG. 14L shows the q-PCR analysis of gene expression in livers from HFD-induced obesity mice treated with control and AF1 peptide, n=4 (AF1 peptide) and 5 (control) mice/group. FIGS. 14M and 14N show the Serum insulin, ALP, ALT, AST, cholesterol, HDL, LDL, triglyceride, and NEFA levels in HFD-induced obesity mice treated with control and AF1 peptide, n=4-5 mice/group. Data are presented as mean±SEM. *p<0.05, ** p<0.01, Students' t test.

FIGS. 15A, 15B, 15C, 15D, 15E, 15F, 15G, 15H, and 15I show that the AF1 peptide improves glucose homeostasis in DKO male and control aging female mice. FIGS. 15A and 15B show the glucose tolerance tests in DKO male mice treated with control and AF1 peptide, n=4 (CNTR) and 6 (DKO-CNTR and DKO-AF1 peptide) mice/group. FIG. 15C shows the insulin tolerance test in DKO male mice treated with control and AF1 peptide, n=4 (CNTR) and 6 (DKO-CNTR and DKO-AF1 peptide) mice/group. * DKO-CNTR vs IRS1/IRS2F/F-CNTR, #DKO-AF1 peptide vs DKO-CNTR. FIG. 15D shows the effect of AF1 peptide on insulin sensitivity in livers from DKO male mice treated with control and AF1 peptide, n=3 mice/group. Representative blots were shown. FIG. 15E shows the effect of AF1 peptide on insulin sensitivity in eWAT from DKO male mice treated with control and AF1 peptide, n=3 mice/group. Representative blots were shown. FIG. 15F shows the effect of AF1 peptide on insulin sensitivity in skeleton muscle from DKO male mice treated with control and AF1 peptide, n=3 mice/group. Representative blots were shown. FIGS. 15G and 15H show the glucose tolerance tests in aging female mice (12-month-old) treated with AF1 peptide for 5 weeks, n=5 mice/group. FIG. 15I show the insulin tolerance tests in aging female mice (12-month-old) treated with AF1 peptide for 5 weeks, n=5 mice/group. Data are presented as mean±SEM. * p<0.05, ** p<0.01, *** p<0.001, **** p<0.0001; #p<0.05, Students' t test (FIGS. 15G-15I), One-way ANOVA (FIGS. 15A-15F).

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

Reference also is made herein to peptides, polypeptides, proteins and compositions comprising peptides, polypeptides, and proteins. As used herein, a polypeptide and/or protein is defined as a polymer of amino acids, typically of length>100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110). The terms peptide or polypeptide can be used interchangeably to refer to a polymer of amino acids, of a length typically of 20-100 amino acids.

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

The term “variant” means a polypeptide derived from a parent polypeptide or protein by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X’, the insertion may be at position ‘X+1’ (‘downstream’) or at position ‘X−1’ (‘upstream’).

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant polypeptide may have substantially the same functional activity as a reference polypeptide. For example, a variant polypeptide may exhibit one or more biological activities associated with binding a ligand and/or binding DNA at a specific binding site.

Variants comprising a fragment of a reference amino acid sequence are contemplated herein. A “fragment” is a portion of an amino acid sequence which is identical in sequence to but shorter in length than the reference sequence. A fragment may comprise up to the entire length of the reference sequence, minus at least one amino acid residue. For example, a fragment may comprise from 5 to 1000 contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous amino acid residues of a reference polynucleotide or reference polypeptide, respectively. Fragments may be preferentially selected from certain regions of a molecule, for example the N-terminal region and/or the C-terminal region of a polypeptide. The term “at least a fragment” encompasses the full-length polypeptide.

The term “administer”, “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The terms “prevent,” “preventing,” “prevention,” and grammatical variations thereof as used herein, refer to a method of partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

As used herein, by a “subject” means an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating or impeding one or more causes of a disorder or condition. Treatments according to the disclosure may be applied preventively, prophylactically, palliatively, or remedially. Treatments are administered to a subject prior to onset (e.g., before obvious signs of disease), during early onset (e.g., upon initial signs and symptoms of disease), or after an established development of disease. Prophylactic administration can occur for several days to years prior to the manifestation of symptoms of an infection.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

A “nucleic acid” is a chemical compound that serves as the primary information-carrying molecules in cells and makes up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The term “therapeutically effective” refers to the amount of the composition used is of sufficient quantity to ameliorate one or more causes or symptoms of a disease or disorder. Such amelioration only requires a reduction or alteration, not necessarily elimination.

A “therapeutic composition” refers to at least one substance, molecule, or compound suitable for administering to a subject, wherein the composition further includes a pharmaceutical carrier. A non-limiting example includes a therapeutic composition comprises a synthetic estrogen receptor peptide and a sterile water-based solution.

### Estrogen Receptor Synthetic Peptides and Compositions

Diabetes mellitus, specifically diabetes mellitus type 2, is a systemic disease characterized by an imbalance in energy metabolism, primarily caused by insulin dysfunction. The estrogen receptors, estrogen receptor alpha (ERα) and estrogen receptor beta (ERβ), have been found to regulate energy metabolism specifically in insulin-sensitive tissues, including, but not limited to muscle, liver, and adipose tissue. Current treatments to manage and/or treat Type II diabetes often lead to insulin resistance and worsening of the disease. Therefore, improved compositions and treatments are needed to overcome insulin resistance in subjects with Type II diabetes.

In one aspect, disclosed herein is a synthetic peptide comprising a peptide from an activation function 1 (AF1) domain of an estrogen receptor alpha (ERα) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids. In some embodiments, the peptide from the AF1 domain of the ERα protein comprises 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more amino acids.

In some embodiments, the at least 30 amino acids from the AF1 domain of the ERα protein are selected from the first 60 amino acids of the ERα protein. In some embodiments, the 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 amino acids from the AF1 domain of the ERα protein are selected from the first 60 amino acids of the ERα protein. In some embodiments, the at least 30 amino acids from the AF1 domain of the ERα protein are selected from the first 60 amino acids of SEQ ID NO: 7. In some embodiments, the at least 30 amino acids from the AF1 domain of the ERα protein are selected from the first 60 amino acids of SEQ ID NO: 9.

In some embodiments, the synthetic peptide comprises at least 70%, at least 75%, at least 80%, at least 85%, at least 90%, at least 95%, at least 99% or at least 100% identity to SEQ ID NO: 1. In some embodiments, the synthetic peptide comprises at least 70% identity to SEQ ID NO: 1. In some embodiments, the synthetic peptide comprises at least 75% identity to SEQ ID NO: 1. In some embodiments, the synthetic peptide comprises at least 80% identity to SEQ ID NO: 1. In some embodiments, the synthetic peptide comprises at least 85% identity to SEQ ID NO: 1. In some embodiments, the synthetic peptide comprises at least 90% sequence identity to SEQ ID NO: 1. In some embodiments, the synthetic peptide comprises at least 95% identity to SEQ ID NO: 1. In some embodiments, the synthetic peptide comprises at least 99% sequence identity to SEQ ID NO: 1. In some embodiments, the synthetic peptide comprises SEQ ID NO: 1.

In some embodiments, the AF1 domain of the ERα protein comprises SEQ ID NO: 2, or a variant or fragment thereof. In some embodiments, the cell penetrating peptide comprises SEQ ID NO: 3, or a variant or fragment thereof.

In some embodiments, the AF1 domain of the ERα protein comprises at least 70%, at least 75%, at least 80%, at least 85%, at least 90%, at least 95%, at least 99% or at least 100% identity to SEQ ID NO: 2. In some embodiments, the AF1 domain of the ERα protein comprises at least 70% identity to SEQ ID NO: 2. In some embodiments, the AF1 domain of the ERα protein comprises at least 75% identity to SEQ ID NO: 2. In some embodiments, the AF1 domain of the ERα protein comprises at least 80% identity to SEQ ID NO: 2. In some embodiments, the AF1 domain of the ERα protein comprises at least 85% identity to SEQ ID NO: 2. In some embodiments, the AF1 domain of the ERα protein comprises at least 90% sequence identity to SEQ ID NO: 2. In some embodiments, the AF1 domain of the ERα protein comprises at least 95% identity to SEQ ID NO: 2. In some embodiments, the AF1 domain of the ERα protein comprises at least 99% sequence identity to SEQ ID NO: 2. In some embodiments, the AF1 domain of the ERα protein comprises SEQ ID NO: 2.

In some embodiments, the cell penetrating peptide comprises at least 70%, at least 75%, at least 80%, at least 85%, at least 90%, at least 95%, at least 99% or at least 100% identity to SEQ ID NO: 3. In some embodiments, the cell penetrating peptide comprises at least 70% identity to SEQ ID NO: 3. In some embodiments, the cell penetrating peptide comprises at least 75% identity to SEQ ID NO: 3. In some embodiments, the cell penetrating peptide comprises at least 80% identity to SEQ ID NO: 3. In some embodiments, the cell penetrating peptide comprises at least 85% identity to SEQ ID NO: 3. In some embodiments, the cell penetrating peptide comprises at least 90% sequence identity to SEQ ID NO: 3. In some embodiments, the cell penetrating peptide comprises at least 95% identity to SEQ ID NO: 3. In some embodiments, the cell penetrating peptide comprises at least 99% sequence identity to SEQ ID NO: 3. In some embodiments, the cell penetrating peptide comprises SEQ ID NO: 3.

In some embodiments, the cell penetrating peptide comprises a transactivator of transcription (TAT) peptide.

In some embodiments, the synthetic peptide is further linked to a light emitting molecule. In some embodiments, the light emitting molecule comprises a fluorescent molecule. In some embodiments, the light emitting molecule comprises a fluorescein molecule, or a derivative thereof. In some embodiments, the light emitting molecule comprises fluorescein isothiocyanate (FITC). In some embodiments, the light emitting molecule comprises a succinimidyl ester modified fluorescein or NHS-fluorescein. In some embodiments, the light emitting molecule comprises a carboxyfluorescein. In some embodiments, the light emitting molecule comprises a carboxyfluorescein succinimidyl ester. In some embodiments, the light emitting molecule comprises a pentafluorophenyl esters (PFP). In some embodiments, the light emitting molecule comprises a tetrafluorophenyl esters (TFP).

In one aspect, disclosed herein is a pharmaceutical composition for enhancing insulin sensitivity comprising a synthetic peptide comprising a peptide from an activation function 1 (AF1) domain of an estrogen receptor alpha (ERα) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids, and a pharmaceutically acceptable carrier.

In one aspect, disclosed herein is a pharmaceutical composition for enhancing insulin sensitivity comprising the synthetic peptide of any preceding aspect, wherein the peptide comprises a pharmaceutically acceptable carrier.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

In some embodiments, the peptide from the AF1 domain of the ERα protein comprises 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more amino acids.

The composition may be administered in such amounts, time, and route deemed necessary in order to achieve the desired result. The exact amount of the composition will vary from subject to subject, depending on the species, age, and general condition of the subject, the severity of the disease, the particular composition, its mode of administration, its mode of activity, and the like. The composition is preferably formulated in dosage unit form for ease of administration and uniformity of dosage. It will be understood, however, that the total daily usage of the composition will be decided by the attending physician within the scope of sound medical judgment. The specific therapeutically effective dose level for any particular subject will depend upon a variety of factors including the severity of the disease; the activity of the composition employed; the specific composition employed; the age, body weight, general health, sex and diet of the patient; the time of administration, route of administration, and rate of excretion of the specific composition employed; the duration of the treatment; drugs used in combination or coincidental with the specific composition employed; and like factors well known in the medical arts.

The composition may be administered by any route. In some embodiments, the composition is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the composition (e.g., its stability in the environment of the gastrointestinal tract), the condition of the subject (e.g., whether the subject is able to tolerate oral administration), etc.

The exact amount of a composition required to achieve a therapeutically effective amount will vary from subject to subject, depending on species, age, and general condition of a subject, severity of the side effects or disease, identity of the particular compound(s), mode of administration, and the like. The amount to be administered to, for example, a child or an adolescent can be determined by a medical practitioner or person skilled in the art and can be lower or the same as that administered to an adult.

### Synthetic Peptide Vectors

In one aspect, disclosed herein is a nucleic acid encoding the synthetic peptide of any preceding aspect.

In one aspect, disclosed herein is a vector comprising a nucleic acid encoding the synthetic peptide of any preceding aspect.

As used herein, “vector” refers to a nucleic acid construct of a plasmid or a virus or viral vector. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector encoding the synthetic peptide is a naked DNA or is comprised in a nanoparticle (e.g., liposomal vesicle, porous silicon nanoparticle, gold-DNA conjugate particle, polyethyleneimine polymer particle, cationic peptides, etc.).

### Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

### Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson, Curr Opin Mol Ther 5:633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

In some embodiments, the vector comprises one or more nucleic acid sequences encoding the ERα synthetic peptide, an AF1 domain peptide, cell penetrating peptide, or any combination thereof. In some embodiments, the vector comprises SEQ ID NO: 4, SEQ ID NO: 5, SEQ ID NO: 6, SEQ ID NO: 8, SEQ ID NO: 10, or any combination thereof.

In some embodiments, the vector of any preceding aspect comprises at least 70% sequence identity to SEQ ID NO: 4. In some embodiments, the vector of any preceding aspect comprises at least 75% sequence identity to SEQ ID NO: 4. In some embodiments, the vector of any preceding aspect comprises at least 80% sequence identity to SEQ ID NO: 4. In some embodiments, the vector of any preceding aspect comprises at least 85% sequence identity to SEQ ID NO: 4. In some embodiments, the vector of any preceding aspect comprises at least 90% sequence identity to SEQ ID NO: 4. In some embodiments, the vector of any preceding aspect comprises SEQ ID NO: 4.

In some embodiments, the vector of any preceding aspect comprises at least 70% sequence identity to SEQ ID NO: 5. In some embodiments, the vector of any preceding aspect comprises at least 75% sequence identity to SEQ ID NO: 5. In some embodiments, the vector of any preceding aspect comprises at least 80% sequence identity to SEQ ID NO: 5. In some embodiments, the vector of any preceding aspect comprises at least 85% sequence identity to SEQ ID NO: 5. In some embodiments, the vector of any preceding aspect comprises at least 90% sequence identity to SEQ ID NO: 5. In some embodiments, the vector of any preceding aspect comprises SEQ ID NO: 5.

In some embodiments, the vector of any preceding aspect comprises at least 70% sequence identity to SEQ ID NO: 6. In some embodiments, the vector of any preceding aspect comprises at least 75% sequence identity to SEQ ID NO: 6. In some embodiments, the vector of any preceding aspect comprises at least 80% sequence identity to SEQ ID NO: 6. In some embodiments, the vector of any preceding aspect comprises at least 85% sequence identity to SEQ ID NO: 6. In some embodiments, the vector of any preceding aspect comprises at least 90% sequence identity to SEQ ID NO: 6. In some embodiments, the vector of any preceding aspect comprises SEQ ID NO: 6.

In some embodiments, the vector of any preceding aspect comprises at least 70% sequence identity to SEQ ID NO: 8. In some embodiments, the vector of any preceding aspect comprises at least 75% sequence identity to SEQ ID NO: 8. In some embodiments, the vector of any preceding aspect comprises at least 80% sequence identity to SEQ ID NO: 8. In some embodiments, the vector of any preceding aspect comprises at least 85% sequence identity to SEQ ID NO: 8. In some embodiments, the vector of any preceding aspect comprises at least 90% sequence identity to SEQ ID NO: 8. In some embodiments, the vector of any preceding aspect comprises SEQ ID NO: 8.

In some embodiments, the vector of any preceding aspect comprises at least 70% sequence identity to SEQ ID NO: 10. In some embodiments, the vector of any preceding aspect comprises at least 75% sequence identity to SEQ ID NO: 10. In some embodiments, the vector of any preceding aspect comprises at least 80% sequence identity to SEQ ID NO: 10. In some embodiments, the vector of any preceding aspect comprises at least 85% sequence identity to SEQ ID NO: 10. In some embodiments, the vector of any preceding aspect comprises at least 90% sequence identity to SEQ ID NO: 10. In some embodiments, the vector of any preceding aspect comprises SEQ ID NO: 10.

In some embodiments, the vector of any preceding aspect further comprises a nucleic acid sequence encoding a light-emitting molecule. In some embodiments, vector is linked or fused to the light-emitting molecule. In some embodiments, the light emitting molecule comprises a fluorescent molecule, including but not limited to a green fluorescent protein, a yellow fluorescent protein, or any variants thereof. In some embodiments, the light emitting molecule comprises a fluorescein molecule, or a derivative thereof. In some embodiments, the light emitting molecule comprises fluorescein isothiocyanate (FITC). In some embodiments, the light emitting molecule comprises a succinimidyl ester modified fluorescein or NHS-fluorescein. In some embodiments, the light emitting molecule comprises a carboxyfluorescein. In some embodiments, the light emitting molecule comprises a carboxyfluorescein succinimidyl ester. In some embodiments, the light emitting molecule comprises pentafluorophenyl esters (PFP). In some embodiments, the light emitting molecule comprises tetrafluorophenyl esters (TFP).

### Methods

The present disclosure also provides methods using the synthetic peptide derived from an estrogen receptor alpha (ERα) protein to treat/prevent diabetes.

In one aspect, disclosed herein is a method of treating or preventing diabetes in a subject in need thereof, the method comprising administering a therapeutically effective amount of a synthetic peptide comprising: a peptide from an activation function 1 (AF1) domain of an estrogen receptor alpha (ERα) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids. In some embodiments, the peptide from the AF1 domain of the ERα protein comprises 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more amino acids.

In some embodiments, the synthetic peptide comprises at least 70%, at least 75%, at least 80%, at least 85%, at least 90%, at least 95%, at least 99% or at least 100% identity to SEQ ID NO: 1. In some embodiments, the synthetic peptide comprises at least 70% identity to SEQ ID NO: 1. In some embodiments, the synthetic peptide comprises at least 75% identity to SEQ ID NO: 1. In some embodiments, the synthetic peptide comprises at least 80% identity to SEQ ID NO: 1. In some embodiments, the synthetic peptide comprises at least 85% identity to SEQ ID NO: 1. In some embodiments, the synthetic peptide comprises at least 90% sequence identity to SEQ ID NO: 1. In some embodiments, the synthetic peptide comprises at least 95% identity to SEQ ID NO: 1. In some embodiments, the synthetic peptide comprises at least 99% sequence identity to SEQ ID NO: 1. In some embodiments, the synthetic peptide comprises SEQ ID NO: 1.

In some embodiments, the synthetic peptide interacts with an insulin receptor substrate (IRS) to activate insulin signaling in the subject. In some embodiments, the IRS comprises an insulin receptor substrate 1 (IRS1).

In some embodiments, the synthetic peptide enhances insulin sensitivity in the subject.

In some embodiments, the AF1 domain comprises SEQ ID NO: 2, or a variant or fragment thereof.

In some embodiments, the cell penetrating peptide comprises SEQ ID NO: 3, or a variant or fragment thereof.

In some embodiments, the ER is an estrogen receptor α (ERα) protein. In some embodiments, the cell penetrating peptide comprises a transactivator of transcription (TAT) peptide. In some embodiments, the cell penetrating peptide directs the synthetic peptide into a cell. In some embodiments, the cell is an insulin-sensitive cell. In some embodiments, the cell is a liver cell, a muscle cell, or an adipose cell.

In some embodiments, the synthetic peptide is further linked to a light emitting molecule. In some embodiments, the light emitting molecule comprises a fluorescent molecule. In some embodiments, the light emitting molecule comprises a fluorescein molecule, or a derivative thereof. In some embodiments, the light emitting molecule comprises fluorescein isothiocyanate (FITC). In some embodiments, the light emitting molecule comprises a succinimidyl ester modified fluorescein or NHS-fluorescein. In some embodiments, the light emitting molecule comprises a carboxyfluorescein. In some embodiments, the light emitting molecule comprises a carboxyfluorescein succinimidyl ester. In some embodiments, the light emitting molecule comprises pentafluorophenyl esters (PFP). In some embodiments, the light emitting molecule comprises tetrafluorophenyl esters (TFP).

In one aspect, disclosed herein is a method of enhancing, increasing, and/or improving insulin sensitivity in a subject, the method comprising administering a therapeutically effective amount of a synthetic peptide comprising: a peptide from an activation function 1 (AF1) domain of an estrogen receptor alpha (ERα) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids. In some embodiments, the peptide from the AF1 domain of the ERα protein comprises 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more amino acids.

In some embodiments, the synthetic peptide comprises at least 70%, at least 75%, at least 80%, at least 85%, at least 90%, at least 95%, at least 99% or at least 100% identity to SEQ ID NO: 1. In some embodiments, the synthetic peptide comprises at least 70% identity to SEQ ID NO: 1. In some embodiments, the synthetic peptide comprises at least 75% identity to SEQ ID NO: 1. In some embodiments, the synthetic peptide comprises at least 80% identity to SEQ ID NO: 1. In some embodiments, the synthetic peptide comprises at least 85% identity to SEQ ID NO: 1. In some embodiments, the synthetic peptide comprises at least 90% sequence identity to SEQ ID NO: 1. In some embodiments, the synthetic peptide comprises at least 95% identity to SEQ ID NO: 1. In some embodiments, the synthetic peptide comprises at least 99% sequence identity to SEQ ID NO: 1. In some embodiments, the synthetic peptide comprises SEQ ID NO: 1.

In some embodiments, the synthetic peptide interacts with an insulin receptor substrate (IRS) to activate insulin signaling and increase insulin sensitivity in the subject. In some embodiments, the IRS comprises an insulin receptor substrate 1 (IRS1).

In some embodiments, the AF1 domain comprises SEQ ID NO: 2, or a variant or fragment thereof.

In some embodiments, the cell penetrating peptide comprises SEQ ID NO: 3, or a variant or fragment thereof.

In some embodiments, the ER is an estrogen receptor α (ERα) protein.

In some embodiments, the cell penetrating peptide comprises a transactivator of transcription (TAT) peptide. In some embodiments, the cell penetrating peptide is located at the amino terminus (N-terminal) end of the synthetic peptide. In some embodiments, the cell penetrating peptide directs the synthetic peptide into a cell. In some embodiments, the cell is an insulin-sensitive cell. In some embodiments, the cell is a liver cell, a muscle cell, or an adipose cell.

In some embodiments, the synthetic peptide is further linked to a light emitting molecule. In some embodiments, the light emitting molecule comprises a fluorescent molecule. In some embodiments, the light emitting molecule comprises a fluorescein molecule, or a derivative thereof. In some embodiments, the light emitting molecule comprises fluorescein isothiocyanate (FITC). In some embodiments, the light emitting molecule comprises a succinimidyl ester modified fluorescein or NHS-fluorescein. In some embodiments, the light emitting molecule comprises a carboxyfluorescein. In some embodiments, the light emitting molecule comprises a carboxyfluorescein succinimidyl ester. In some embodiments, the light emitting molecule comprises pentafluorophenyl esters (PFP). In some embodiments, the light emitting molecule comprises tetrafluorophenyl esters (TFP).

In some embodiments, the synthetic peptide treats or prevents a type of diabetes in the subject.

In some embodiments, the type of diabetes is Type II diabetes.

In one aspect, disclosed herein is a method of preventing, decreasing, ameliorating, inhibiting, and/or treating insulin resistance in a subject, the method comprising administering a therapeutically effective amount of a synthetic peptide comprising at least 30 amino acids from an activation function (AF) domain of an estrogen receptor α (ERα) protein linked to a cell penetrating peptide, wherein the cell penetrating peptide is linked to the amino terminus (N-terminal) end of the synthetic peptide.

In one aspect, disclosed herein is a method of treating or preventing diet-induced obesity in a subject in need thereof, the method comprising administering a therapeutically effective amount of a synthetic peptide comprising at least 30 amino acids from an activation function (AF) domain of an estrogen receptor α (ERα) protein linked to a cell penetrating peptide, wherein the cell penetrating peptide is linked to the amino terminus (N-terminal) end of the synthetic peptide.

In one aspect, disclosed herein is a method of treating or preventing hepatic steatosis in a subject in need thereof, the method comprising administering a therapeutically effective amount of a synthetic peptide comprising at least 30 amino acids from an activation function (AF) domain of an estrogen receptor α (ERα) protein linked to a cell penetrating peptide, wherein the cell penetrating peptide is linked to the amino terminus (N-terminal) end of the synthetic peptide.

In some embodiments, the method of any preceding aspect comprises administering a therapeutically effective amount of a synthetic peptide comprising 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more amino acids from an activation function (AF) domain of an estrogen receptor α (ERα) protein linked to a cell penetrating peptide, wherein the cell penetrating peptide is linked to the amino terminus (N-terminal) end of the synthetic peptide.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: An Estrogen Receptor α-Derived Peptide Improves Glucose Homeostasis During Obesity

Estrogen improves insulin sensitivity and increases energy expenditure, contributing to sexual dimorphism regarding type 2 diabetes mellitus (T2DM) susceptibility. Estrogen receptor α (ERα) plays a crucial role in mediating estrogen action on glucose and energy homeostasis. However, the underlying mechanisms remain incompletely understood. Here, a ligand-independent effect of ERα on the regulation of glucose homeostasis was found and an ERα-derived peptide was identified as a insulin sensitizer. Deficiency of ERα but not ERβ in the liver impaired glucose homeostasis in male, female, and ovariectomized (OVX) female mice. Mechanistic studies revealed that ERα promoted hepatic insulin sensitivity by suppressing ubiquitination-induced IRS1 degradation. The ERα 1-280 domain mediated the ligand-independent effect of ERα on insulin sensitivity. Furthermore, the present disclosure provides a peptide designed based on ERα 1-280 domain and the present disclosure describes the finding that ERα-derived peptide interacted with IRS1, increased IRS1 stability through suppressing its ubiquitination, and enhanced insulin sensitivity. Importantly, administration of ERα-derived peptide into obese mice significantly increased insulin sensitivity, attenuated glucose intolerance, and improved serum lipid profiles. These findings pave the way for the therapeutic intervention of T2DM by targeting the ligand-independent effect of ERα and indicate that ERα-derived peptide is a potential insulin sensitizer for the treatment of T2DM.

Approximately 415 million people worldwide have diabetes with an estimated 193 million people having undiagnosed diabetes. More than 90% of diabetic patients have type 2 diabetes mellitus (T2DM) that is characterized by insulin resistance and relative insulin deficiency. Insulin resistance is known as an impaired insulin action in target organs, which eventually leads to a relative insulin deficiency. The underlying mechanisms of insulin resistance are complicated, including direct consequence of toxic metabolic by-products accumulation, dysregulation of peptide hormones and inflammatory molecules, as well as activation of intracellular stress response pathways. The antidiabetic medications have been developed by targeting hepatic glucose production (HGP), insulin secretion, and insulin sensitivity during the past several decades. Considering the complexity of diabetes and side-effects of current antidiabetic medicines, it is important to develop the novel therapeutic target of T2DM.

Estradiol-17β (E₂) plays a critical role in the regulation of energy balance and glucose homeostasis, which explain the fact that diabetes is more prevalent in young men than young women. The function of E₂ is mainly mediated through estrogen receptors (ER). Ablation of ERα, but not ERβ, leads to metabolic dysregulation in mice, including increased body weight, enhanced adiposity, and impaired glucose homeostasis. Although the role of E₂-ERα signaling pathway in energy balance and glucose homeostasis is well-documented, its underlying mechanisms are less known. E₂-ERα signaling pathway functions through a genomic mechanism, where E₂ activates ERα to bind to the estrogen response element (ERE) motif and regulate the transcription of target genes. ERα activated by E₂ also triggers downstream signaling pathway through a non-genomic mechanism, including interaction with cell membrane receptors and activation of protein kinases. A previous study has shown that the rescue of ERα non-genomic function normalizes the dysregulation of energy balance and glucose homeostasis in obese ERα knockout female mice; this suggests that the non-genomic action of E2-ERα signaling plays a pivotal role in the regulation of glucose and energy homeostasis. However, the underlying mechanism of E2-ERα non-genomic action is not fully understood.

It was shown that E₂ suppressed HGP and increased insulin sensitivity through the activation of AKT-FOXO1 signaling pathway. Other study revealed that ERα interacted with and activated p85α regulatory subunit of phosphatidylinositol-3-OH kinase (PI3K) in response to E₂ in endothelial cells. Thus, E₂-ERα-PI3K-AKT-FOXO1 signaling pathway partially mediates the non-genomic effect of E2-ERα on energy balance and glucose homeostasis. In addition, overexpression of ERα significantly promoted insulin sensitivity in absence of E₂; this shows that ERα enhances insulin sensitivity in a ligand-independent manner. Insulin receptor substrate (IRS) 1 and 2 are the key targets of insulin receptor to control glucose homeostasis in response to insulin. Hepatic ablation of IRS1 and IRS2 leads to insulin resistance and hyperglycemia in mice. In breast cancer cells, ERα interacts with IRS1 and IRS2 independent of E2, thereby protecting against ubiquitination-induced degradation. Therefore, we hypothesized that ERα binds to the IRS proteins in a ligand-independent manner, thereby regulating insulin sensitivity and glucose homeostasis. Herein, it was found that hepatic ERα deletion resulted in glucose intolerance in male, female, and ovariectomized (OVX) female mice. Mechanistically, ERα increased IRS1 protein stability through inhibiting the phosphorylation of IRS1 at S302 and ubiquitin-induced degradation. Furthermore, ERα 1-280 mediated the ligand-independent effect of ERα on insulin sensitivity and glucose homeostasis. Finally, a peptide (AF1 peptide) was designed based on amino acid sequence of ERα 1-280 domain and demonstrated that AF1 peptide was an insulin sensitizer to increase insulin sensitivity and improve glucose homeostasis in obese mice.

### Results

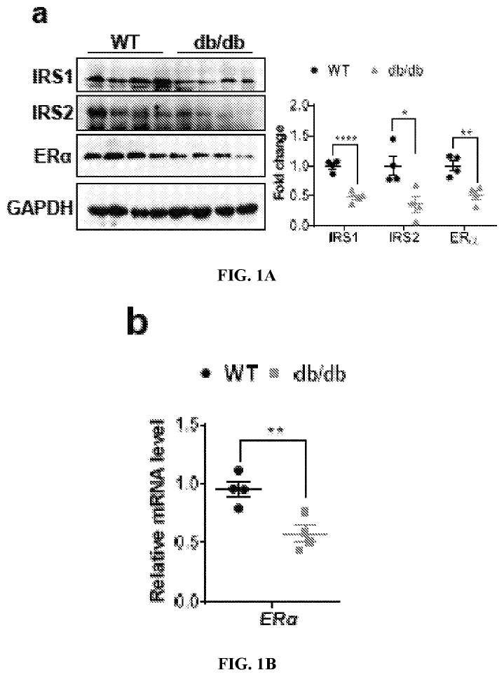

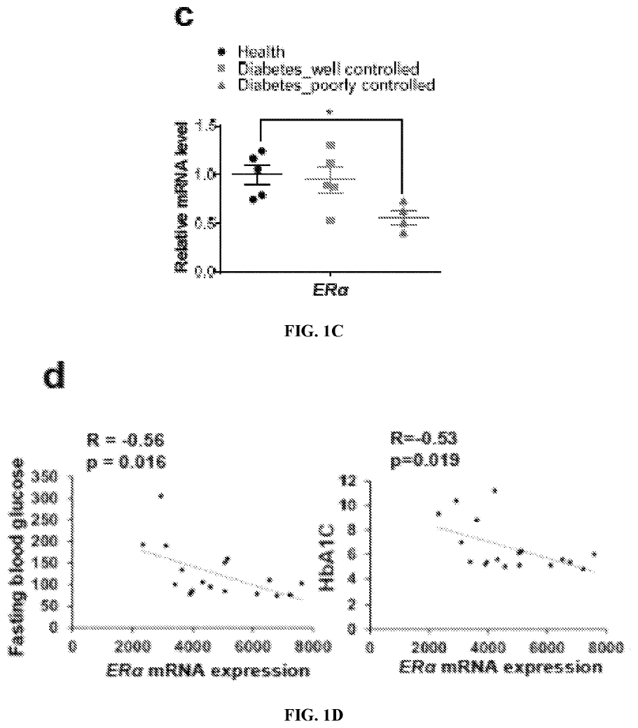

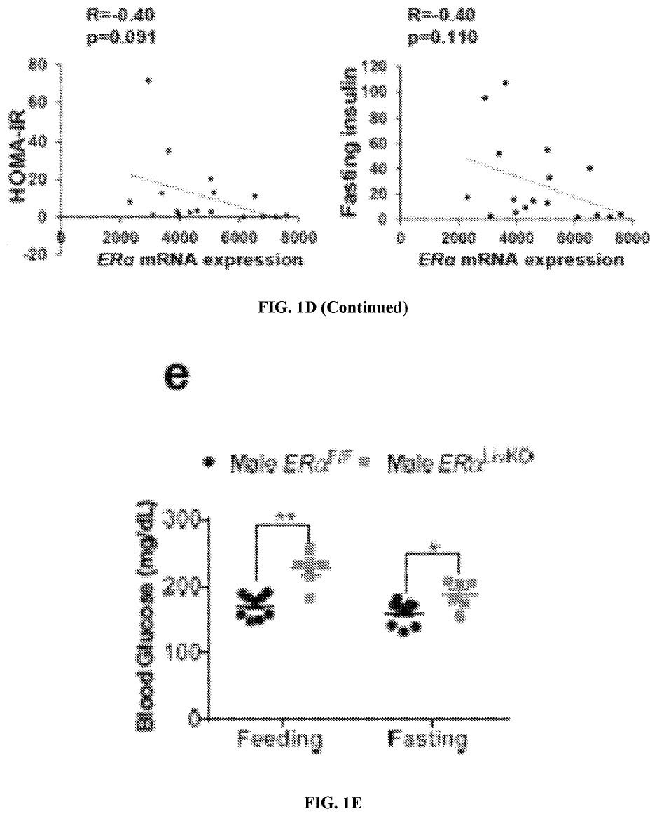

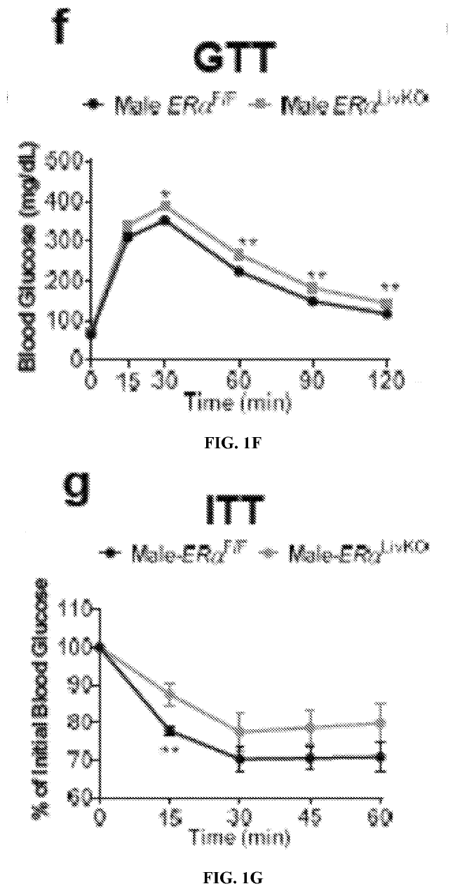

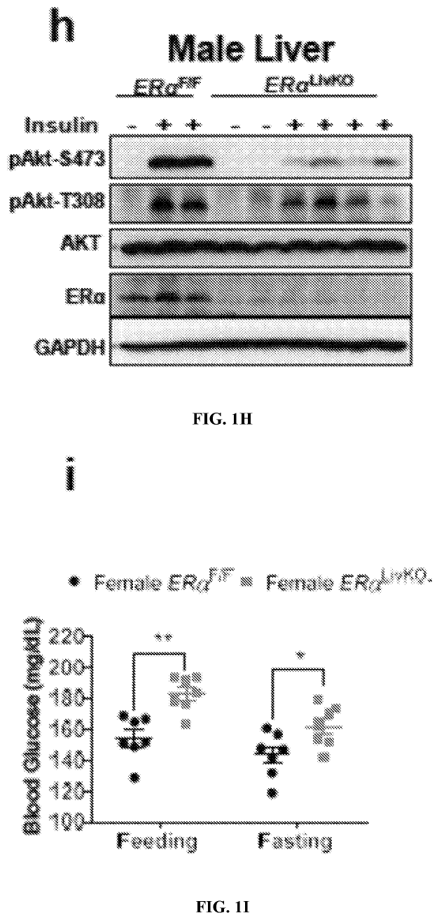

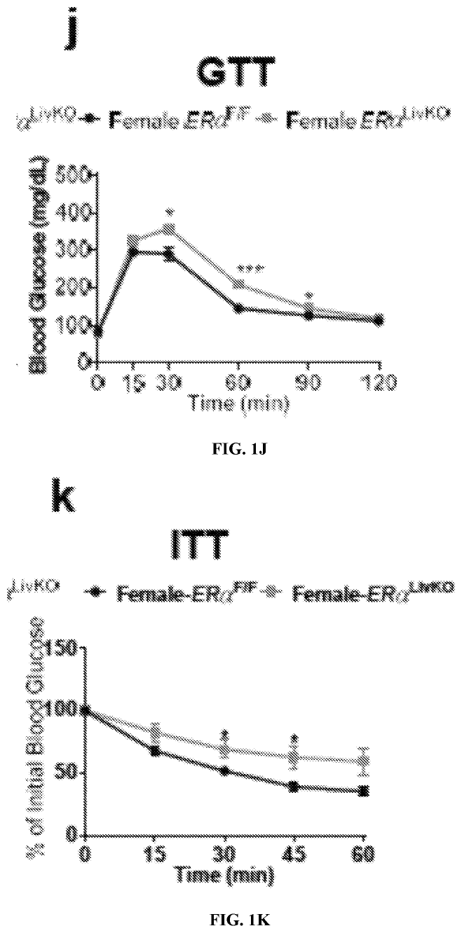

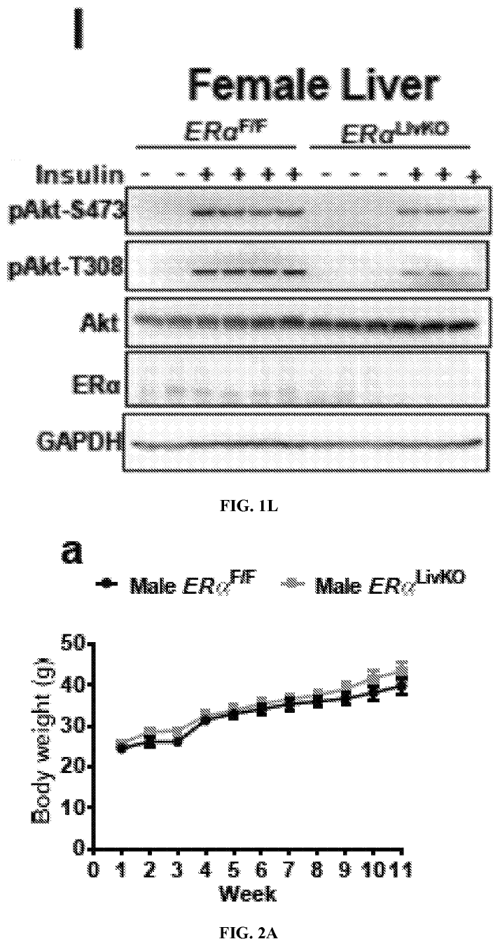

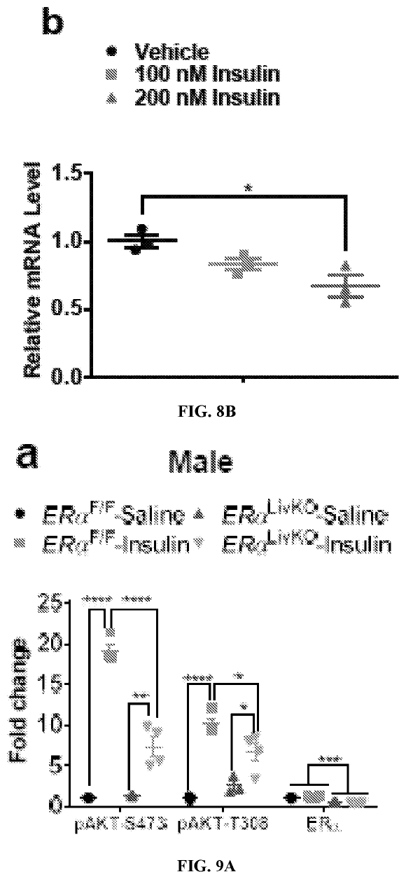

Deletion of hepatic ERα impairs glucose tolerance and insulin sensitivity in both male and female mice. The expression levels of IRS1, IRS2, and ERα were detected in the liver of obese db/db male mice. Compared to wild-type (WT) mice, IRS1, IRS2, and ERα protein levels were significantly decreased by 53%, 64%, and 49%, respectively, in the liver of db/db male mice (FIG. 1A). In addition, the mRNA expression levels of ERα were significantly decreased in the liver of diabetic mice (FIG. 1B). The mRNA expression levels of hepatic ERα were further analyzed in diabetic patients. Interestingly, hepatic ERα mRNA expression levels were significantly decreased in humans with poorly controlled diabetes but not well controlled diabetes (FIG. 1C). Compared to well controlled diabetic individuals, poorly controlled diabetic individuals showed a higher fasting insulin level (FIG. 8A). High dose of insulin treatment (200 nM) significantly decreased mRNA expression levels of ERα in primary hepatocytes (FIG. 8B), showing that decreased hepatic ERα mRNA expression in diabetic patients may be caused by hyperinsulinemia. The gene profiles in the livers of humans were analyzed and hepatic ERα expression levels were negatively associated with fasting blood glucose, HbA1C, HOMA-IR, and fasting insulin (FIG. 1D). These results indicate that hepatic ERα plays an important role in regulating pathogenesis of T2DM.

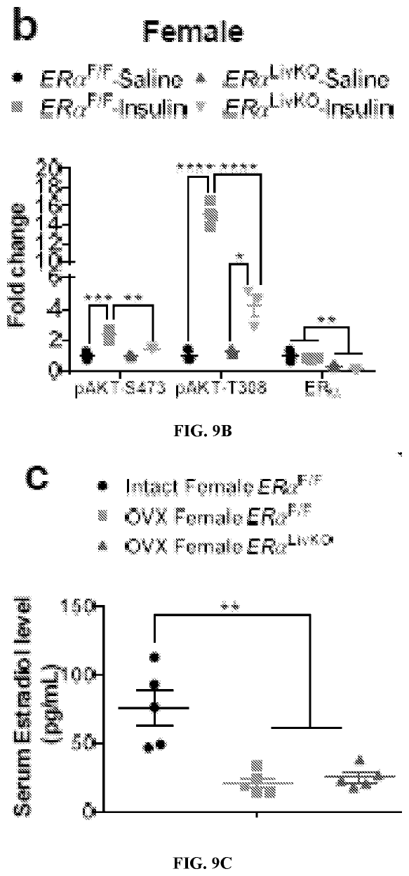

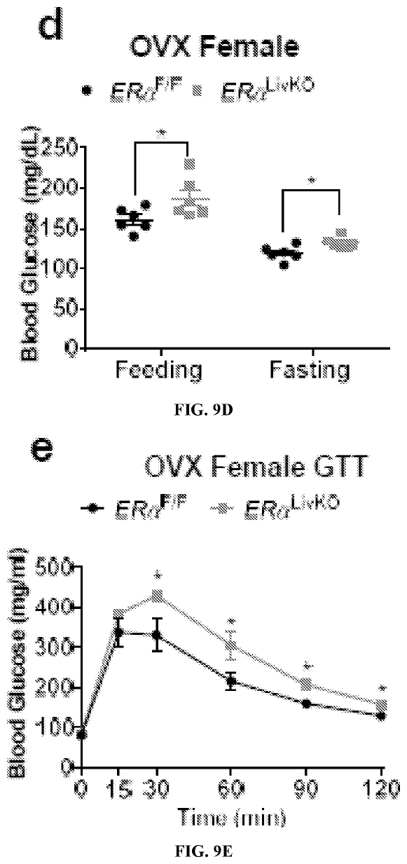

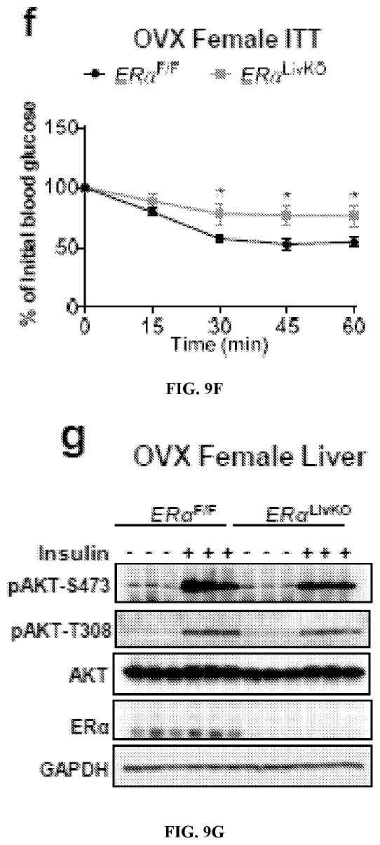

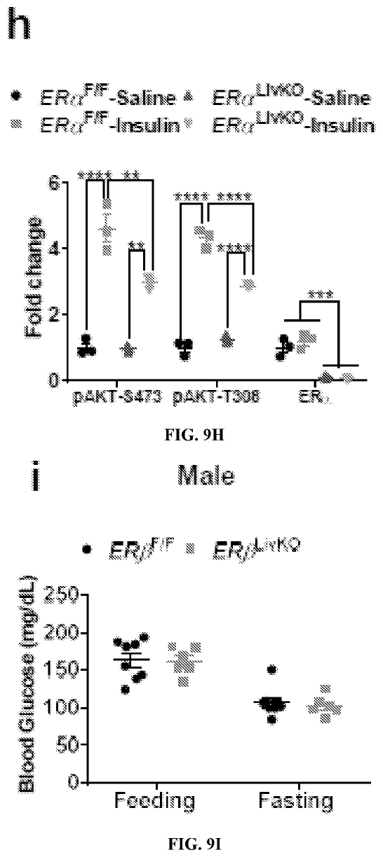

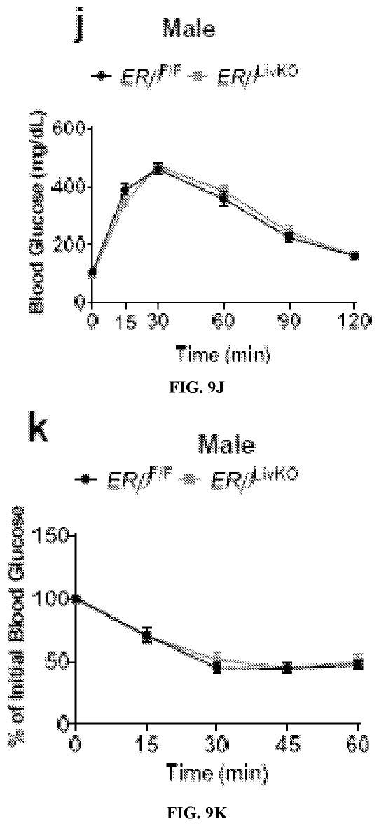

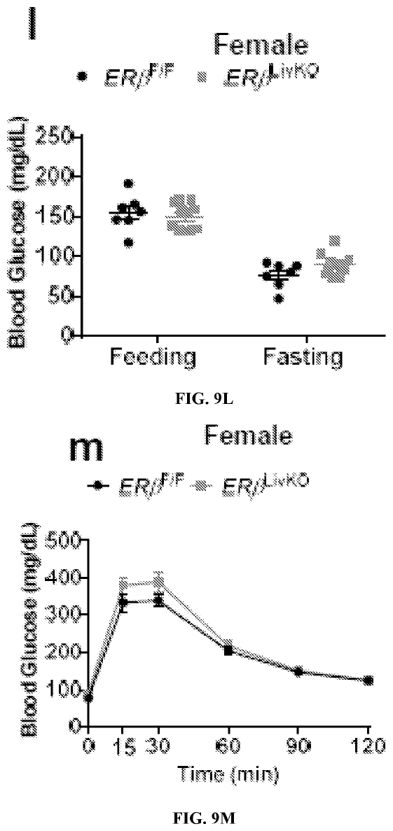

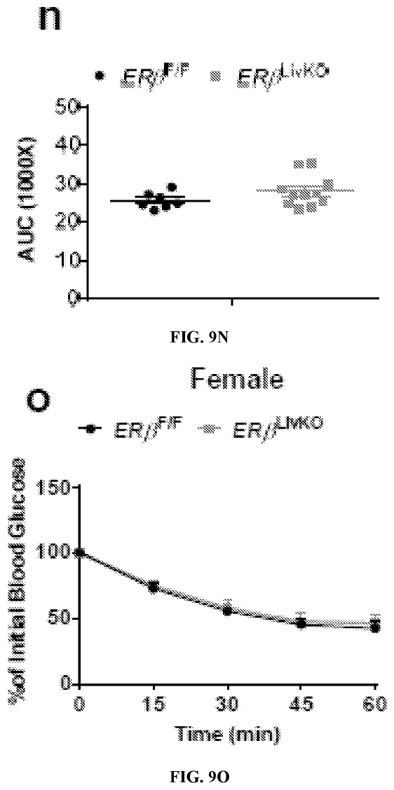

To further detect the effect of hepatic ERα on glucose homeostasis, a liver-specific ERα knockout (ERα^{LivKO}) mice was generated. Glucose tolerance and insulin sensitivity were analyzed in both male and female ERα^{LivKO} mice. Compared to ERα-floxed (ERα^{F/F}) male mice, ERα^{LivKO} male mice showed a 32% increase in random feeding blood glucose (ERα^{F/F}: 170.9±5.7 vs. ERα^{LivKO}; 226.0±10.2 mg/dL, p<0.01) and a 19% increase in 5 h fasting blood glucose (ERα^{F/F}: 160.3±6.0 vs. ERα^{LivKO}: 187±8.4 mg/dL, p<0.05, FIG. 1e). Additionally, glucose tolerance and insulin sensitivity were significantly impaired in ERα^{LivKO} male mice (FIGS. 1F and 1G). Hepatic insulin signaling was significantly impaired in ERα^{LivKO} male mice, indicated by a 62% (p<0.0001) and a 34% (p<0.05) decrease in insulin-induced phosphorylated AKT at S473 (pAKT-S473) and at T308 (pATK-T308), respectively. ERα protein levels were significantly decreased by 60% in the liver of ERα^{LivKO} male mice (p<0.001, FIG. 1H and FIG. 9A). In female mice, hepatic ERα deletion led to a 10% increase in random feeding blood glucose (ERα^{F/F}: 154.8±5.3 vs. ERα^{LivKO}: 183.4±4.2 mg/dL, p<0.01) and a 13% increase in 5 h fasting blood glucose (ERα^{F/F}: 143.7±5.5 vs. ERα^{LivKO}: 162.1±5.1 mg/dL, p<0.05, FIG. 1i). Compared to ERα^{F/F} female mice, glucose tolerance and insulin sensitivity were impaired in ERα^{LivKO} female mice (FIGS. 1J and 1K). Similar to the male mice, ERα^{F/F} female mice showed a significant decrease in insulin-induced AKT phosphorylation at S473 by 37% (p<0.01) and T308 by 72% (p<0.0001) in livers. ERα protein levels were downregulated by 72% (p<0.01) in the liver of ERα^{LivKO} female mice (FIG. 1L and FIG. 9B). An ovariectomy (OVX) was performed in ERα^{F/F} and ERα^{LivKO} female mice. Compared to intact female mice, OVX female mice showed around 70% decrease in circulating E2 levels (FIG. 9C). OVX ERα^{LivKO} female mice showed a 17% increase in random feeding blood glucose (p<0.05) and an 11% increase in 5 h fasting blood glucose (p<0.05, FIG. 9D). Compared to OVX ERα^{F/F} female mice, glucose tolerance and insulin sensitivity were significantly impaired in OVX ERα^{LivKO} female mice (FIGS. 9E and 9F). Hepatic insulin sensitivity was significantly attenuated in OVX ERα^{LivKO} female mice, indicated by a 51% decrease in pAKT-S473 (p<0.01) and 46% decrease in pAKT-T308 (p<0.0001). Hepatic ERα protein levels were significantly decreased by 94% (p<0.001, FIGS. 9G and 9H). Considering the effect of hepatic ERα in male and OVX female mice that have a low circulating estrogen level, the present disclosure contemplates that hepatic ERα regulates glucose homeostasis and insulin sensitivity in male mice through a ligand-independent mechanism.

In addition to ERα, ERβ also mediates the function of estrogen. A hepatic ERβ knockout (ERβ^{LivKO}) mice was generated and its role evaluated in the regulation of glucose homeostasis. Hepatic ERβ knockout had no significant effect on random feeding and overnight fasting blood glucose in both male and female mice (FIGS. 9I and 9L). Compared to control mice, glucose tolerance and insulin sensitivity were not significantly impaired in both ERβ^{LivKO} male and female mice (FIGS. 9J, 9K, 9M, 9N, and 9O). Taken together, these results indicate that hepatic ERα regulates glucose tolerance and insulin sensitivity in a ligand-independent manner in male mice.

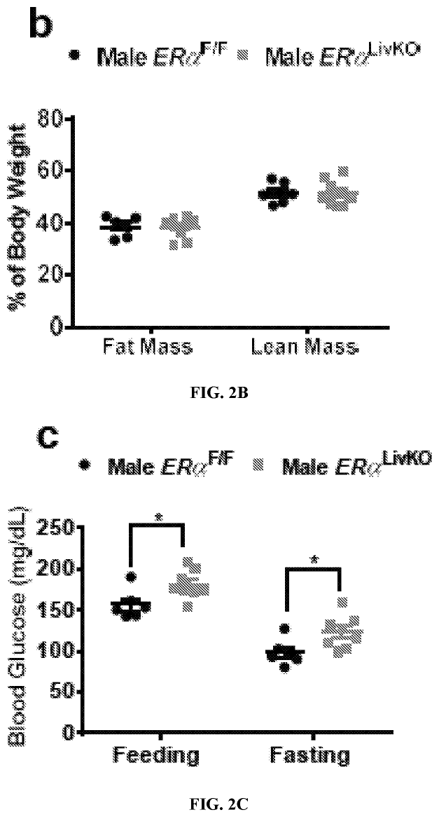

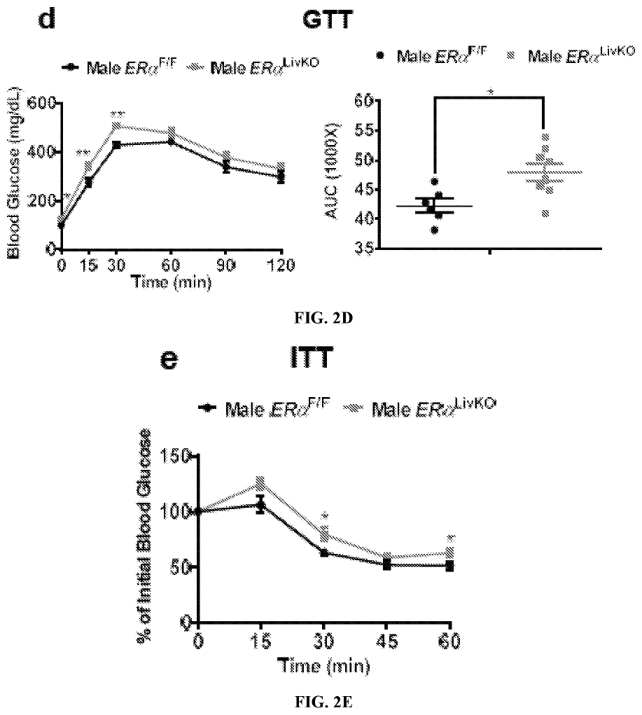

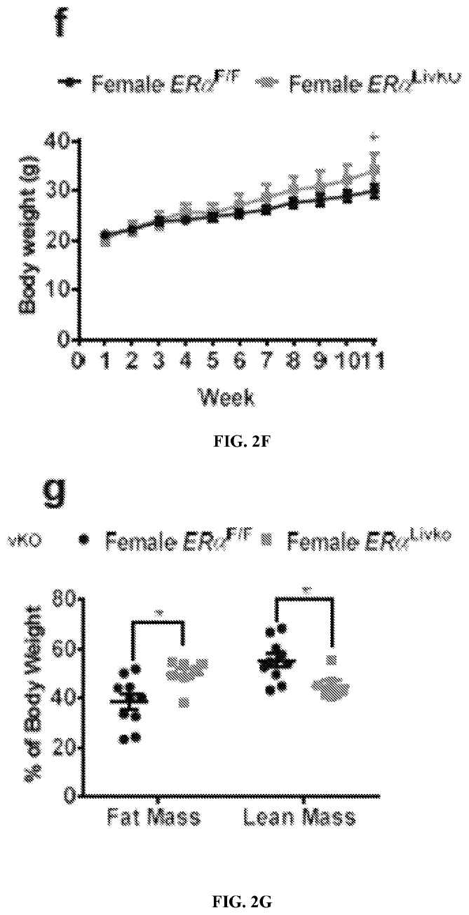

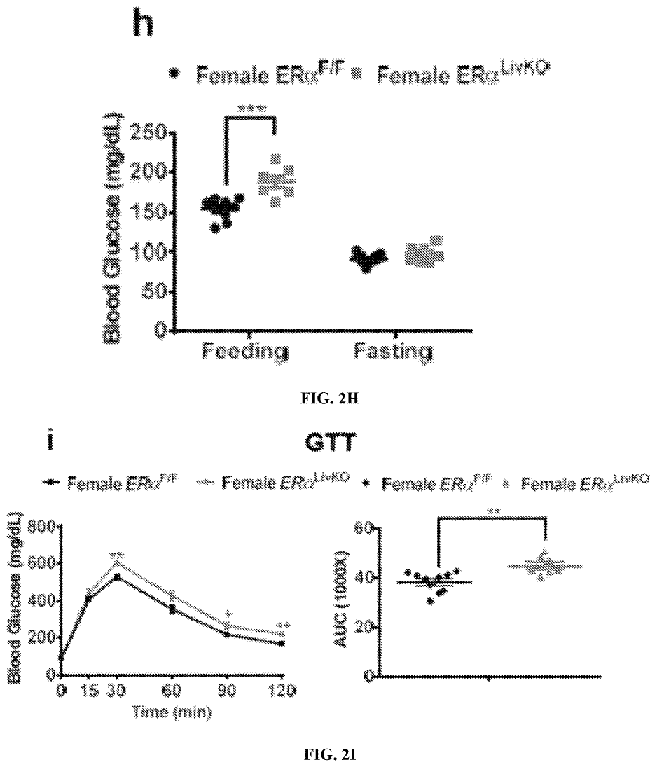

Deletion of hepatic ERα impairs glucose tolerance and insulin sensitivity in both DIO male and female mice. To further detect the role of hepatic ERα in the pathogenesis of diabetes, male and female ERα^{F/F} and ERα^{LivKO} mice were fed a high-fat diet (HFD) for 11 weeks. In diet-induced obese (DIO) male mice, hepatic ERα deficiency did not change body weight and body mass (FIGS. 2A and 2B). Compared to ERα^{F/F} DIO male mice, ERα^{LivKO} DIO male mice showed a 12% increase in random feeding blood glucose and a 25% increase in overnight fasting blood glucose (p<0.05, FIG. 2C). Additionally, glucose tolerance and insulin sensitivity were significantly impaired in ERα^{LivKO} DIO male mice (FIGS. 2D and 2E). In DIO female mice, hepatic ERα deficiency led to a significant increase in body weight at the 11th week of HFD treatment (FIG. 2F). ERα^{LivKO} DIO female mice showed a 30% increase in fat mass and a 19% decrease in lean mass (p<0.05, FIG. 2G). In female DIO mice, hepatic ERα deficiency led to a 22% increase in random feeding blood glucose (p<0.001) and a 6% increase in overnight fasting blood glucose (FIG. 2H). Compared to ERα^{F/F} DIO female mice, glucose tolerance and insulin sensitivity were significantly attenuated in ERα^{LivKO} DIO female mice (FIGS. 2I and 2J). These results indicate that hepatic ERα plays a protective role in diet-induced glucose dysregulation and insulin resistance.

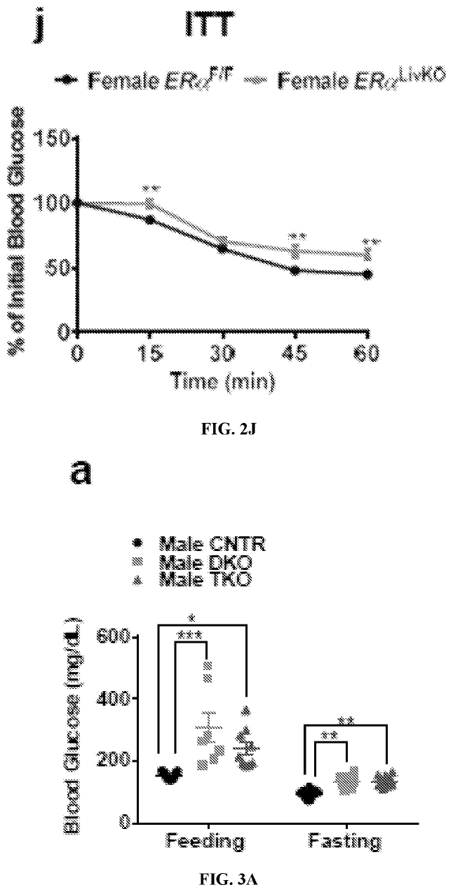

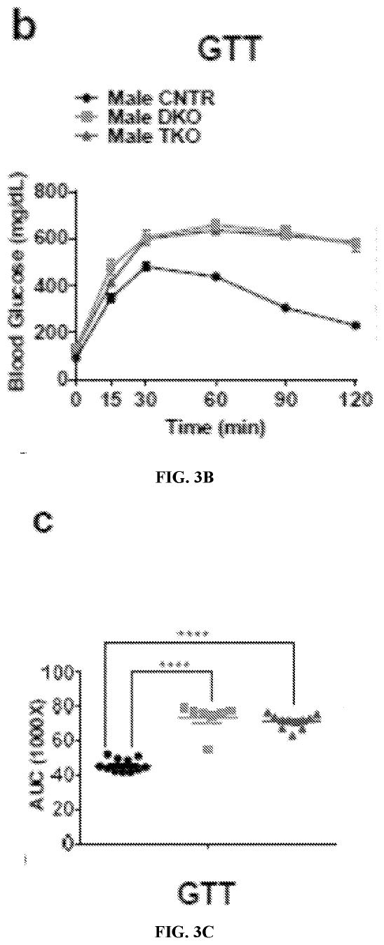

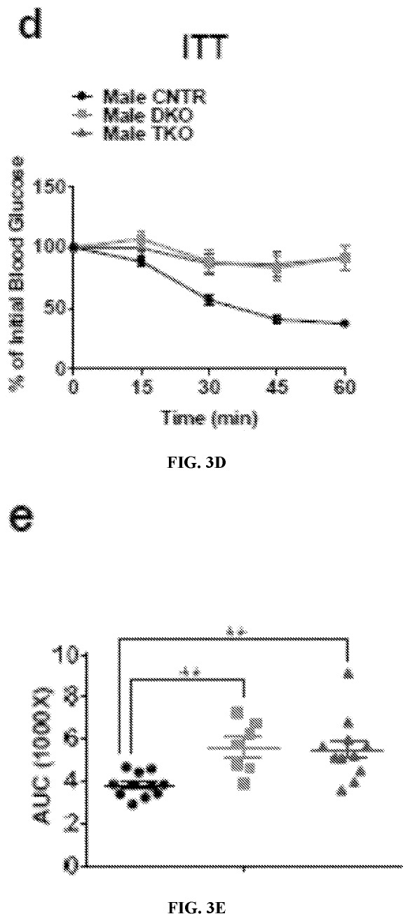

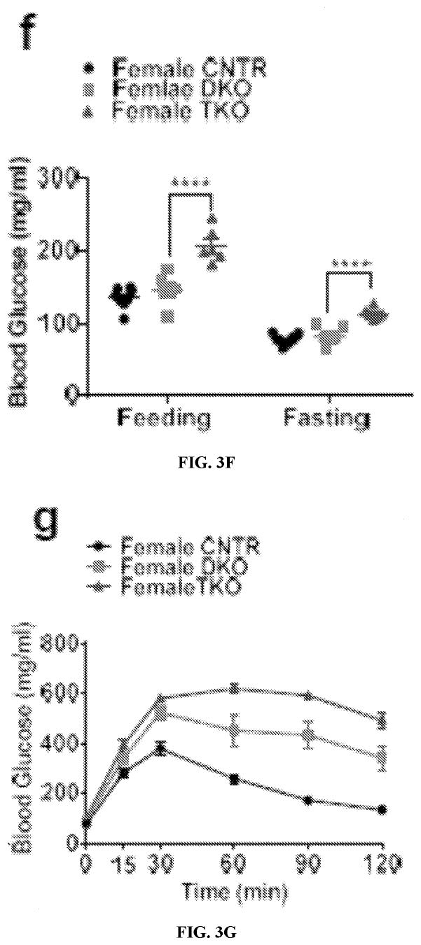

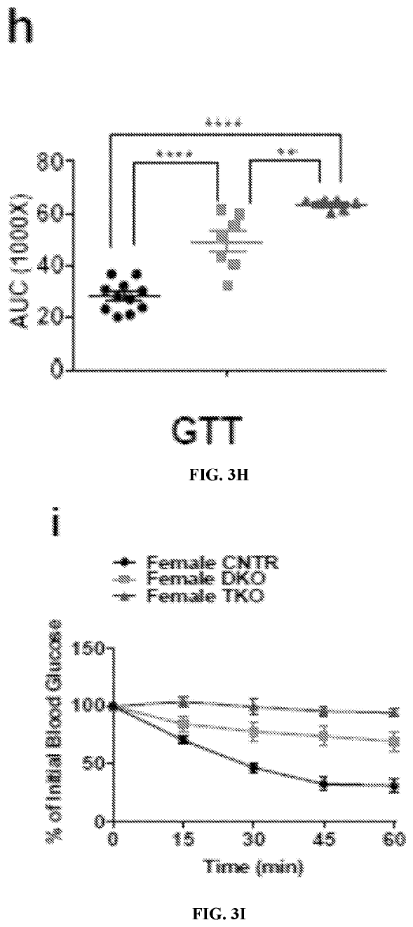

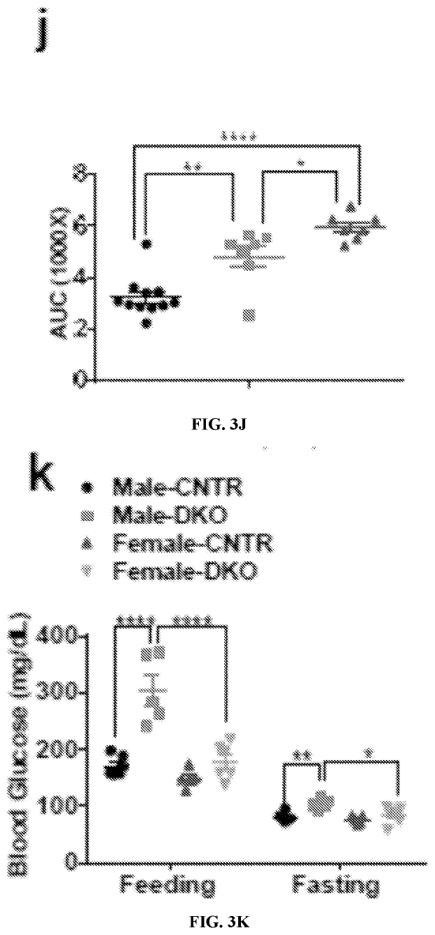

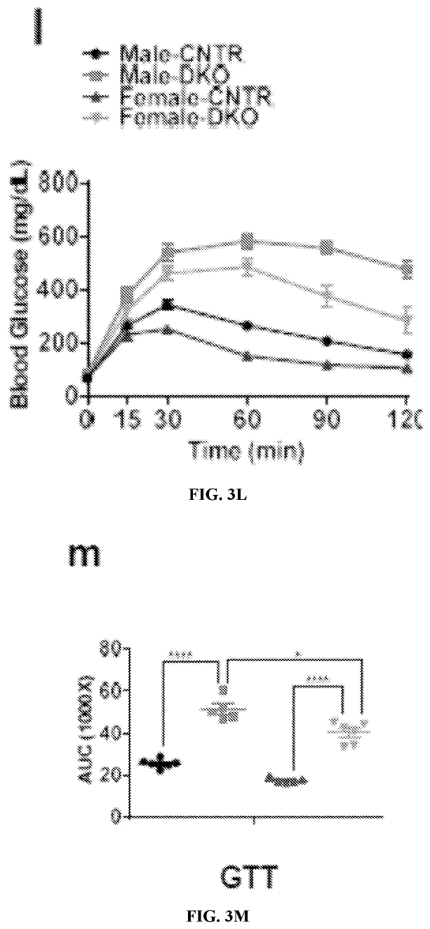

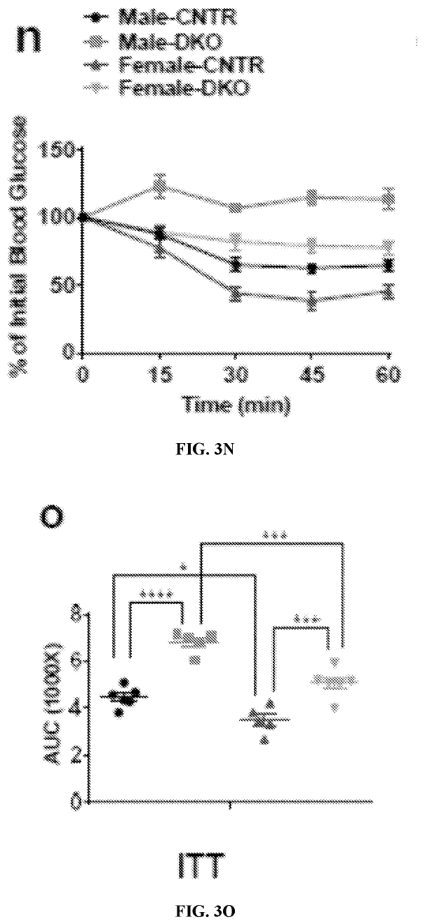

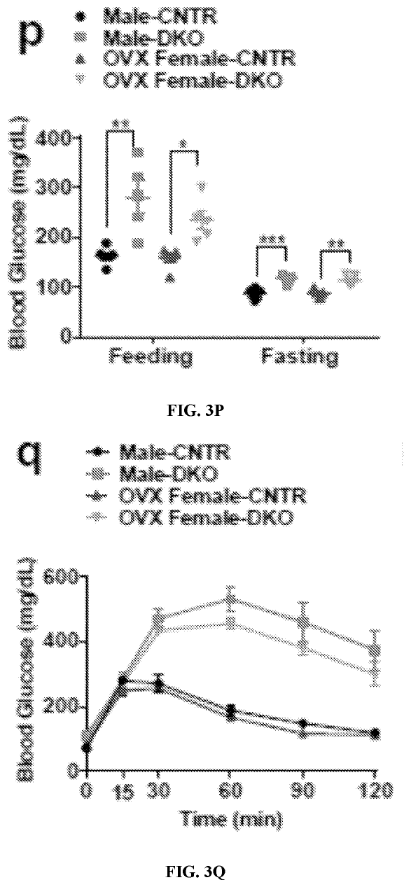

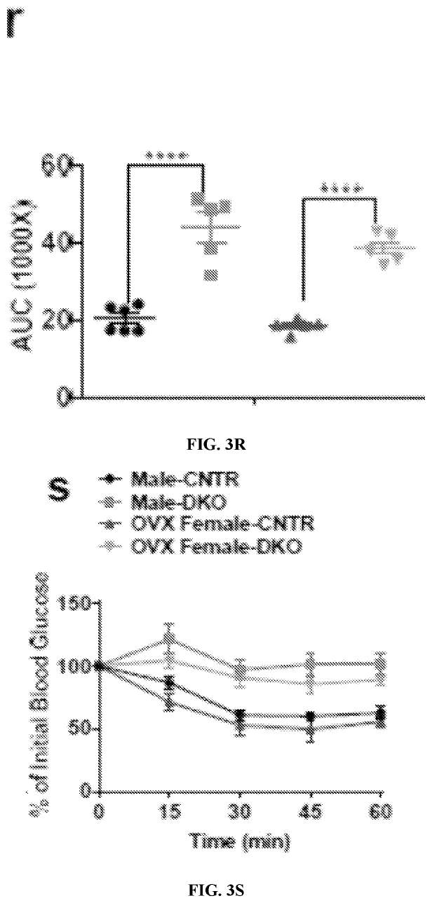

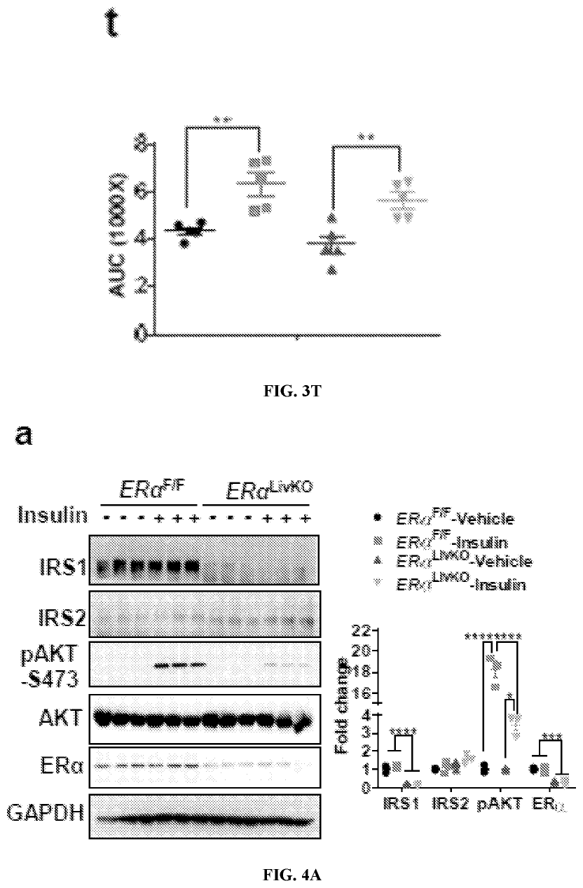

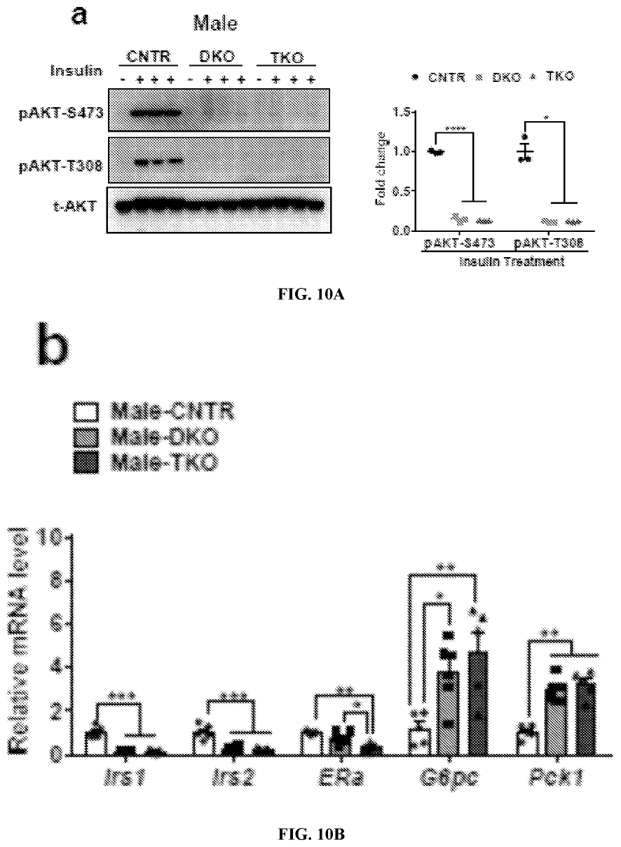

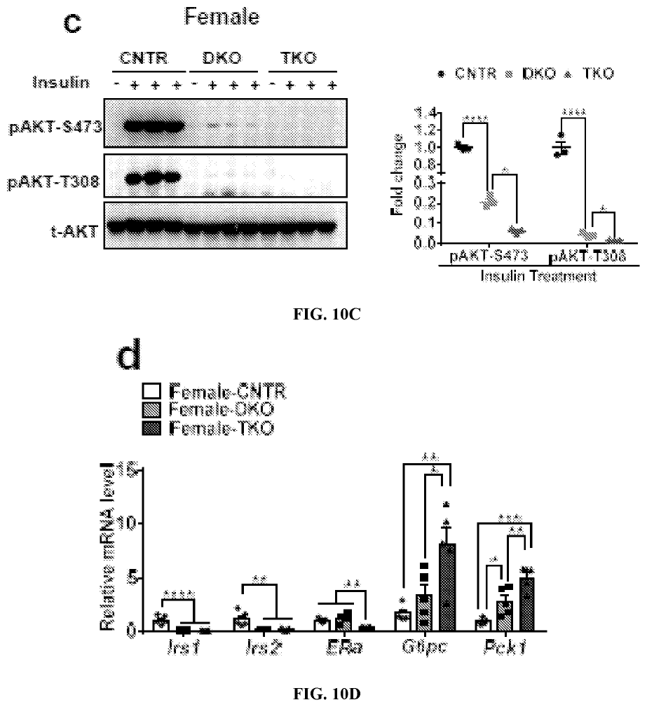

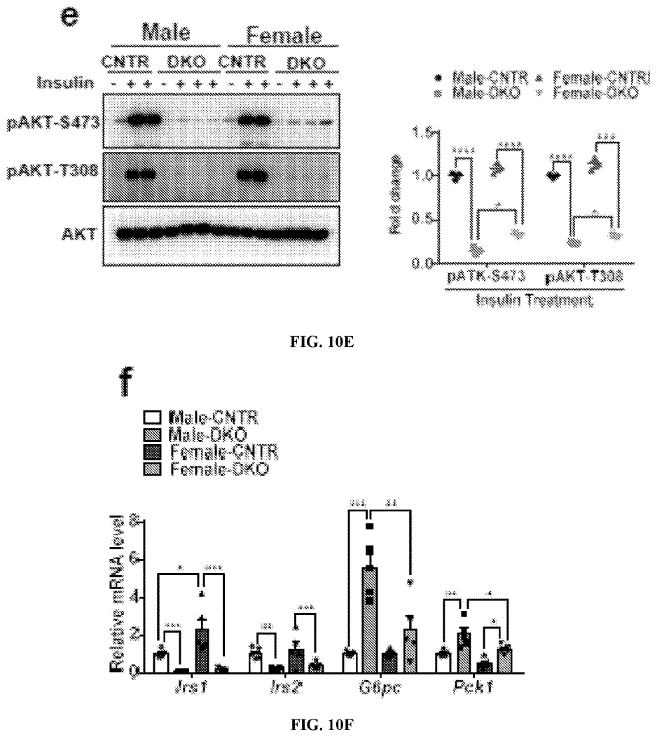

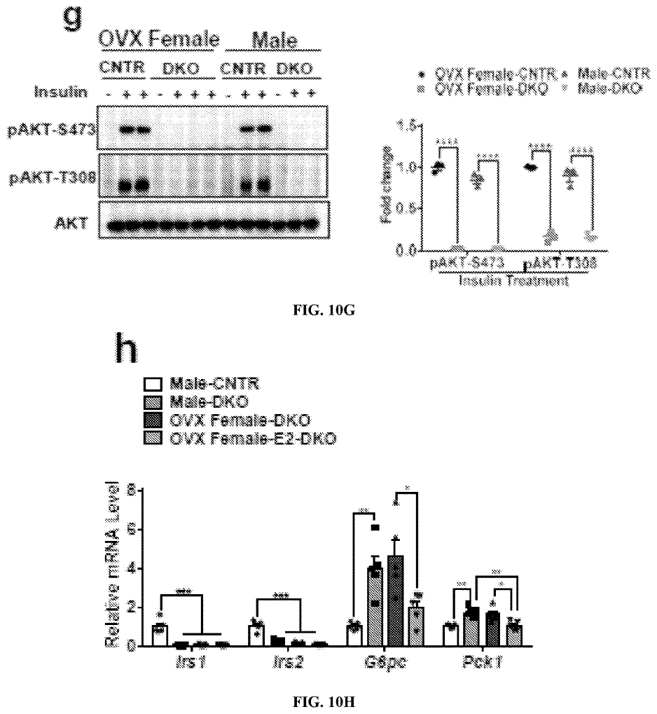

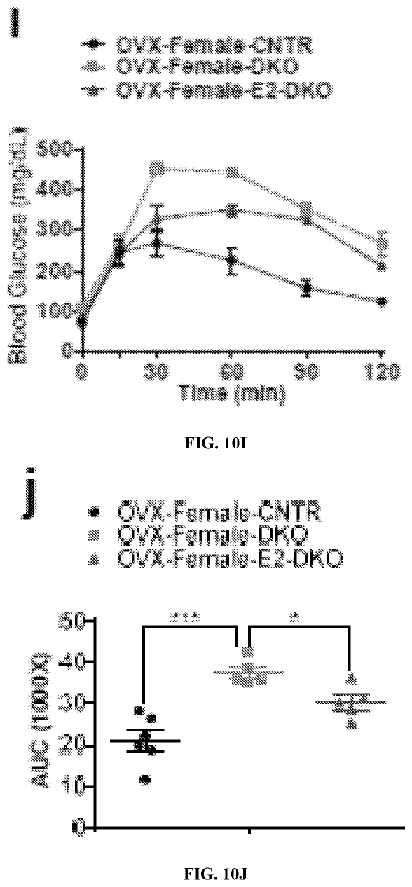

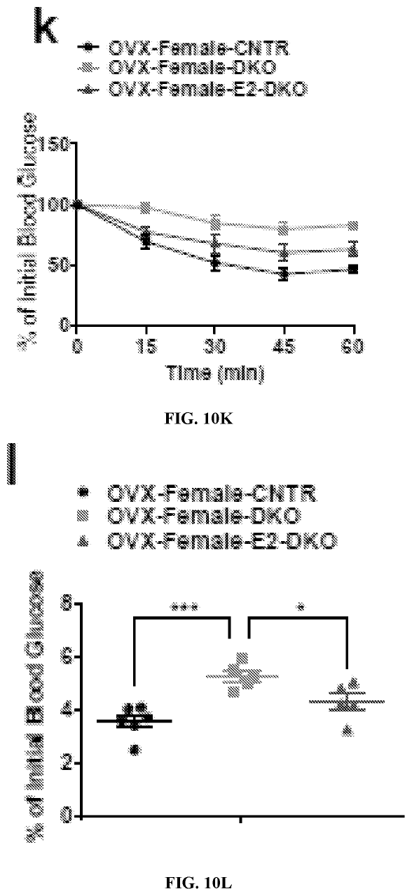

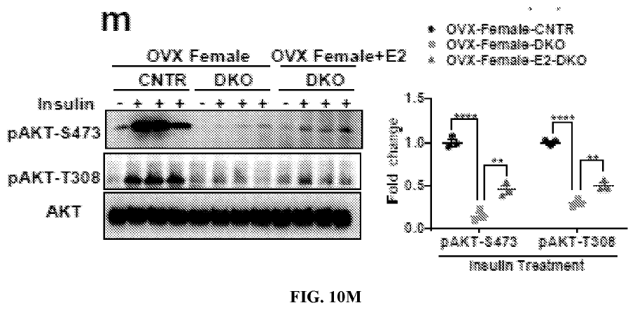

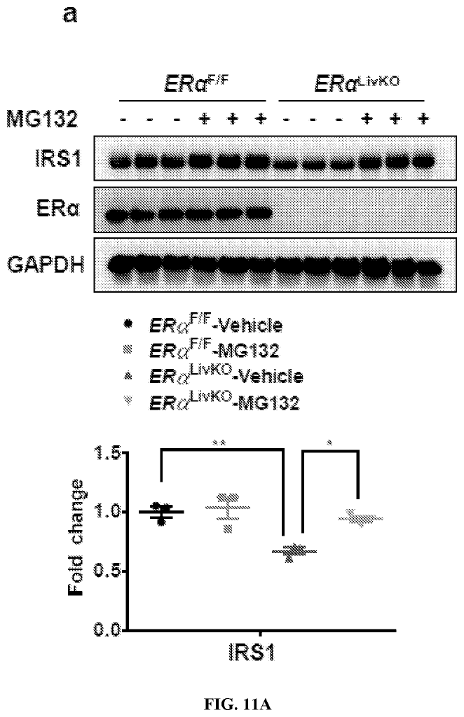

Hepatic ERα regulates glucose tolerance and insulin sensitivity in an IRS1/2-dependent manner in male mice. A previous study showed that ERα protected IRS1 and IRS2 against ubiquitination-induced degradation independent of E₂. Thus, it was reasoned that the ligand-independent effect of ERα on glucose homeostasis may be mediated by IRS1 and IRS2. A liver-specific IRS1 and IRS2 double knockout (DKO) and a liver-specific IRS1, IRS2, and ERα triple knockout (TKO) mice were generated. In male mice, compared to control (CNTR) mice liver IRS1 and ISR2 deletion led to a 102% increase in random feeding blood glucose (CNTR: 151.5±3.5 vs. DKO: 305.6±48.3 mg/dL, p<0.001) and a 33% increase in overnight fasting blood glucose (CNTR: 95.7±3.4 vs. DKO: 127.0±11.6 mg/dL, p<0.01). Hepatic ERα deletion did not further significantly increase random feeding and overnight fasting blood glucose in DKO male mice (FIG. 3A). In addition, DKO male mice showed severe glucose intolerance (p<0.0001) and insulin resistance (p<0.01), as compared to CNTR male mice. Hepatic ERα deletion in DKO male mice had no significant effect on glucose tolerance and insulin sensitivity compared to DKO mice (FIGS. 3B, 3C, 3D, and 3E). Hepatic insulin sensitivity was significantly attenuated in DKO and TKO male mice, indicated by ~86% decreases in insulin-induced AKT phosphorylation at S473 (p<0.0001) and T308 (p<0.05). Compared to DKO male mice, hepatic insulin sensitivity was not significantly affected in TKO male mice (FIG. 10A). Gene expression analysis showed that compared to CNTR male mice, mRNA expression levels of gluconeogenic genes (G6pc and Pck1) were significantly increased by 4.2- and 2.9-fold, respectively, in the livers of DKO and TKO male mice (p<0.01). TKO male mice did not show significant differences in mRNA expression levels of G6pc and Pck1 in liver, as compared to DKO male mice. Expression levels of Irs1, Irs2, and ERα were significantly decreased in DKO and TKO male mouse livers (FIG. 10B). In female mice, liver IRS1 and IRS2 deletion had no significant effects on random feeding (CNTR: 135.3±5.0 vs. DKO: 146.3±9.1 mg/dL) and overnight fasting (CNTR: 77.6±2.5 vs. DKO: 83.2±6.0 mg/dL) blood glucose. However, TKO female mice showed a 45% increase in random feeding blood glucose (TKO: 207.0±9.6 mg/dL, p<0.0001) and 39% increase in overnight fasting blood glucose (TKO: 113.3±3.1 mg/dL, p<0.0001), as compared to DKO female mice (FIG. 3F). Glucose tolerance and insulin sensitivity were significantly impaired in DKO female mice, compared to CNTR female mice. Notably, liver IRS1, IRS2, and ERα triple knockout led to a significant decrease in glucose tolerance (p<0.01) and insulin sensitivity (p<0.05), as compared to DKO female mice (FIGS. 3G, 3H, 3I, and 3J). Additionally, DKO female mice showed a 79% and 95% reduction in insulin-induced AKT phosphorylation at S473 and T308, respectively (p<0.0001). Liver ERα deletion in DKO mice further decreased insulin-induced pAKT-S473 and pAKT-T308 by 70% and 62% (p<0.05), respectively, as compared to DKO female mice (FIG. 10C). DKO female mice showed an increasing trend in the mRNA expression levels of G6pc and Pck1 in the livers, as compared to CNTR female mice. Hepatic ERα deletion significantly increased mRNA expression levels of G6pc by 1.7-fold (p<0.05) and Pck1 by 1.1-fold (p<0.01) in the livers of DKO female mice. The expression levels of Irs1, Irs2, and ERα were significantly decreased in the livers of DKO and TKO female mice (FIG. 10D).

To further investigate the sex difference in DKO mice, glucose homeostasis was analyzed in male and female DKO mice. Consistently, DKO male mice showed significant increases in random feeding (p<0.0001) and overnight fasting (p<0.01) blood glucose compared to CNTR male mice. On the other hand, compared to CNTR female mice, DKO female mice did not show significant increases in blood glucose under both random feeding and overnight fasting conditions. Compared to DKO male mice, DKO female mice showed a 41% decrease in random feeding blood glucose (Male DKO: 306.2±26.9 vs. Female DKO: 178.7±11.3 mg/dL, p<0.0001) and 18% decrease in overnight fasting blood glucose (Male DKO: 103.8±4.5 vs. Female DKO: 83.3±6.1 mg/dL, p<0.05, FIG. 3K). CNTR female mice showed better glucose tolerance and insulin sensitivity than CNTR male mice. However, glucose tolerance and insulin resistance were significantly impaired in both DKO male and female mice. Compared to DKO male mice, glucose tolerance and insulin sensitivity were significantly improved by 21% (p<0.05) and 25% (p<0.001), respectively, in DKO female mice (FIGS. 3L, 3M, 3N, and 3O). In the liver, insulin sensitivity was significantly impaired in both DKO male and female mice. DKO female mice showed an improvement in hepatic insulin sensitivity compared to that in DKO male mice, indicated by a 123% and a 35% increase in insulin-induced pAKT-S473 and pAKT-T308, respectively (p<0.05, FIG. 10E). Consistently, the mRNA expression levels of G6pc and Pck1 were significantly increased in the livers of DKO male mice compared to CNTR male mice. The DKO female mice showed a significant decrease in mRNA expression levels of G6pc by 54% (p<0.01) and Pck1 by 39% (p<0.05), as compared to DKO male mice (FIG. 10F). To test whether the sex difference in glucose homeostasis in DKO mice is mediated by ovary-secreted hormones, such as estrogen, we performed ovariectomy (OVX) in DKO female mice. Consistent with results from DKO male mice, DKO OVX female mice showed significant increases in both random feeding (CNTR OVX: 157.2±9.6 vs. DKO OVX: 234.2±18.8 mg/dL, p<0.05) and overnight fasting (CNTR OVX: 86.2±3.9 vs. DKO OVX: 114.4±4.4 mg/dL, p<0.01) blood glucose (FIG. 3P). Both DKO male and OVX female mice exhibited severe glucose intolerance (p<0.0001) and insulin resistance (p<0.01) compared to CNTR mice. However, there were no significant differences in both glucose tolerance and insulin sensitivity between DKO male and OVX female mice (FIGS. 3Q, 3R, 3S, and 3T). Consistently, hepatic insulin signaling was diminished in both DKO male and OVX female mice (p<0.0001) and significant differences were not observed in insulin-stimulated ATK phosphorylation between DKO male and OVX female mouse livers (FIG. 10G). Consistently, DKO OVX female mice showed comparable mRNA expression levels of G6pc and Pck1 in the livers, as compared to DKO male mice (FIG. 10H). Of note, E2 supplement significantly improved glucose tolerance (p<0.05) and insulin sensitivity (p<0.05) in the DKO OVX female mice (FIGS. 10I, 10J, 10K, and 10L). Consistently, hepatic insulin signaling was significantly enhanced by E2 supplement in the DKO OVX female mice (FIG. 10M). The mRNA expression levels of G6pc (p<0.05) and Pck1 (p<0.05) were significantly decreased by E2 supplement in the livers of DKO OVX female mice (FIG. 10H). These results indicate that the effect of hepatic ERα on insulin sensitivity is mediated through IRS1 and IRS2 in male mice, while in female mice, ovary hormone estrogen regulates insulin sensitivity independent of IRS1 and IRS2. Taken together, IRS1 and IRS2 are required by the ligand-independent effect of ERα on insulin sensitivity in male mice.

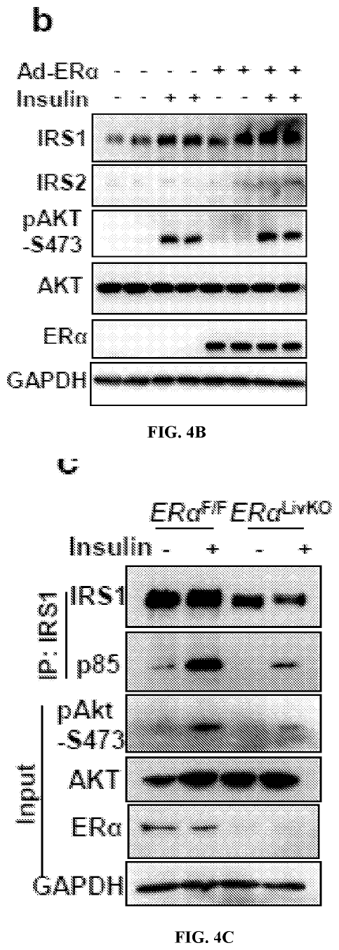

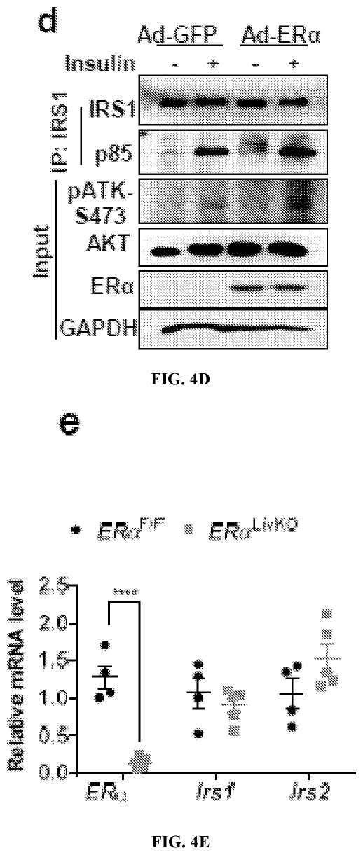

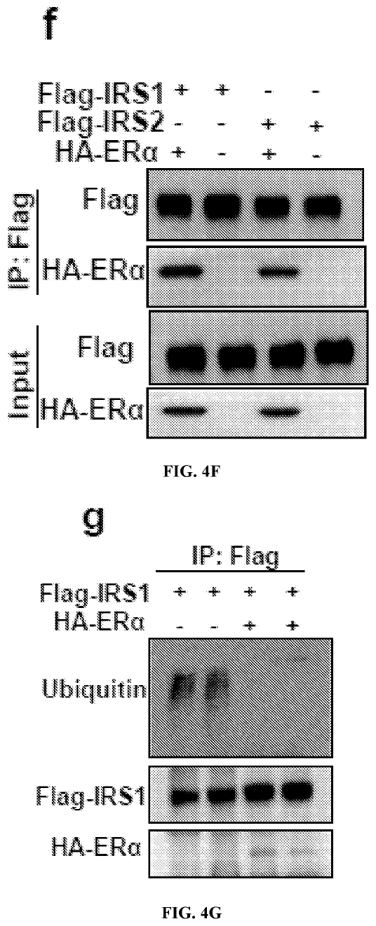

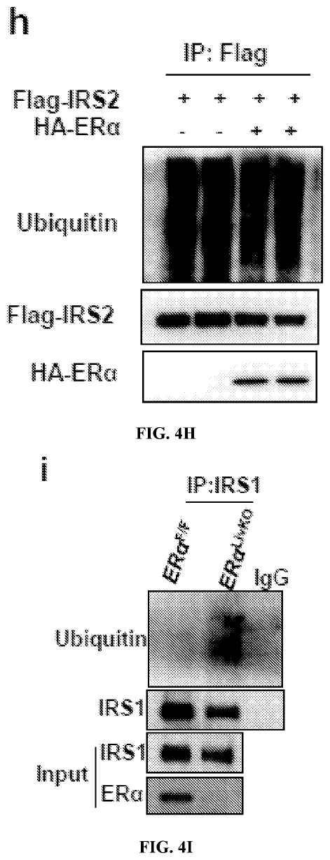

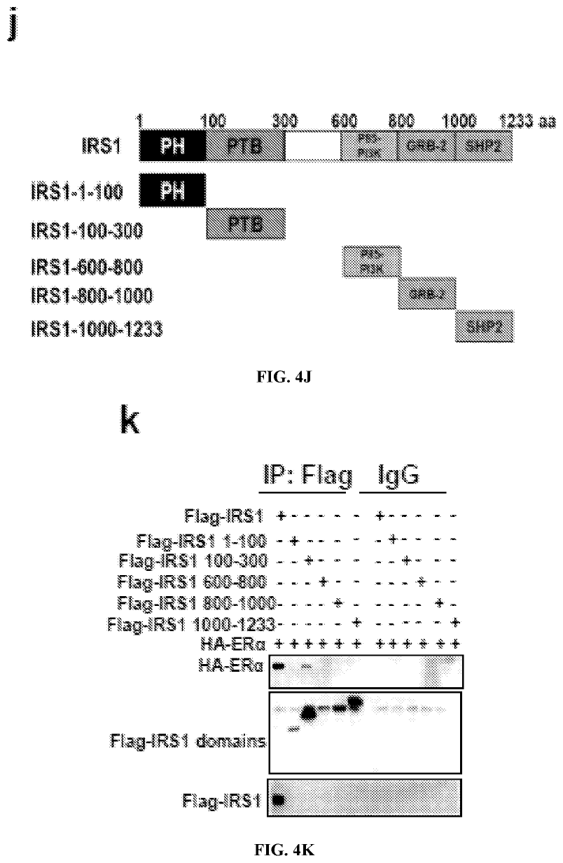

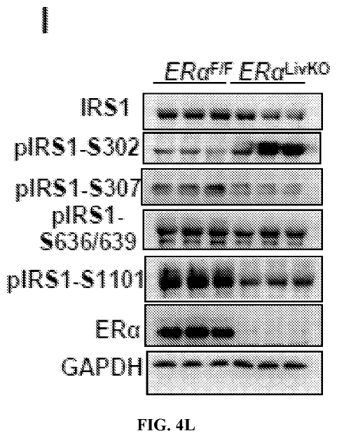

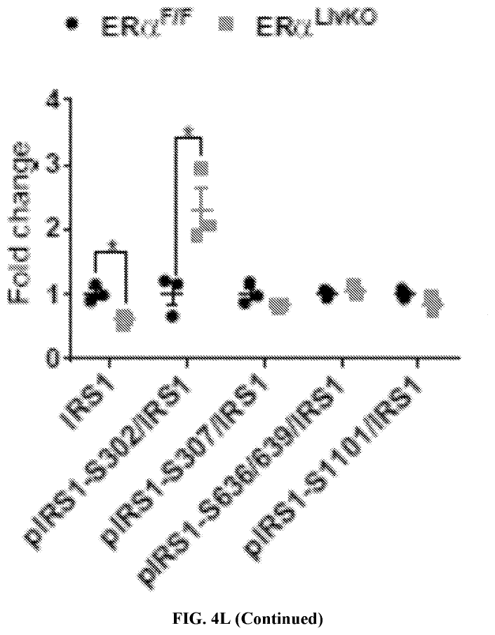

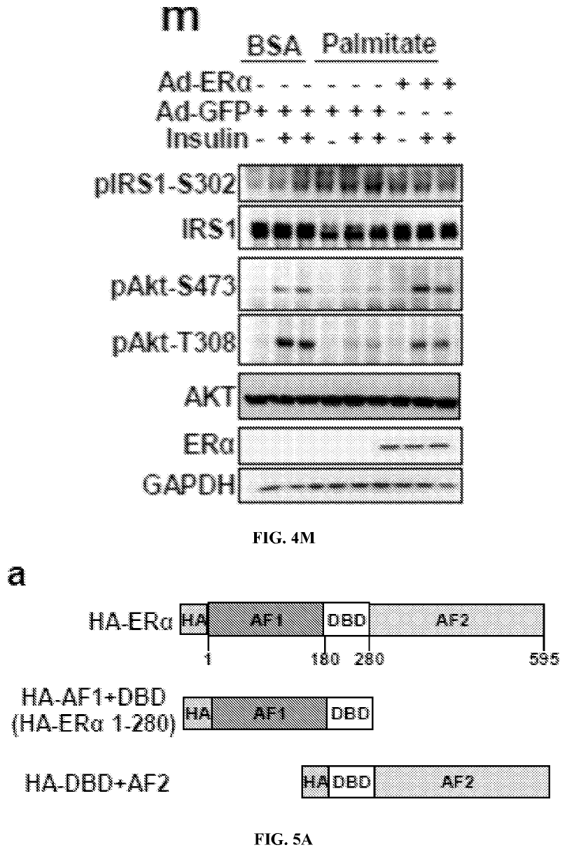

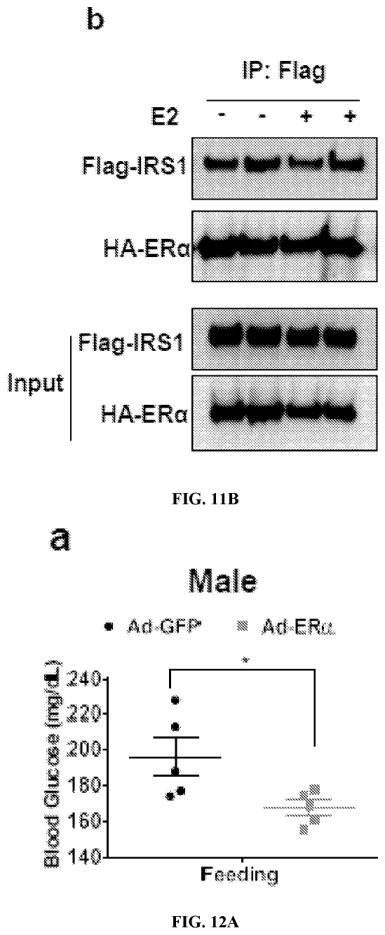

ERα stimulates hepatic insulin signaling through increasing IRS1 protein stability. ERα regulation of the expression of IRS1 and IRS2 to improve hepatic insulin sensitivity was also explored. In mouse primary hepatocytes, ERα deletion decreased IRS1 protein levels by 80% (p<0.0001) but had no significant effect on IRS2 protein abundance. Insulin-stimulated AKT phosphorylation at S473 was diminished by 81% (P<0.0001) in ERα^{LivKO} hepatocytes (FIG. 4A). ERα gain-of-function increased IRS1 and pAKT-S473 protein levels but had a limited effect on IRS2 protein levels in control primary hepatocytes (FIG. 4B). Insulin-induced interaction between IRS1 and p85 was attenuated in ERα^{LivKO} hepatocytes (FIG. 4C). ERα overexpression in control hepatocytes enhanced the interaction between IRS1 and p85 (FIG. 4D). Compared to control hepatocytes, Irs1 and Irs2 mRNA levels were not significantly affected in ERα^{LivKO} hepatocytes (FIG. 4E), which indicates that ERα regulates IRS1 protein expression through a post-translational modification. To test this, control and ERα^{LivKO} hepatocytes were treated with MG132, a proteasome inhibitor and it was found that ERα deficiency significantly decreased IRS1 protein levels by 30% (p<0.01) and MG132 treatment significantly increased IRS1 proteins levels (p<0.05) in ERα^{LivKO} hepatocytes (FIG. 11A). ERα interacted with both IRS1 and IRS2 (FIG. 4F). ERα largely attenuated the ubiquitination of IRS1 but had a limited effect on IRS2 ubiquitination (FIGS. 4G and 4H). Consistently, ERα deletion increased IRS1 ubiquitination in primary hepatocytes (FIG. 4I). E2 treatment had no effect on the interaction between IRS1 and ERα (FIG. 11B), showing that ERα interacts with IRS1 in a ligand-independent manner. To further investigate how ERα regulates IRS1 ubiquitination, plasmids of truncated IRS1 domains were constructed based on its functional unit (FIG. 4J). ERα interacted with the IRS1 100-300 domain (FIG. 4K). Furthermore, the effect of ERα on the serine phosphorylation of IRS1 leads to ubiquitin-mediated degradation. ERα deletion decreased IRS1 protein levels by 39% (p<0.05) and stimulated IRS1-S302 phosphorylation by 130% (p<0.05) in hepatocytes. However, IRS1 phosphorylation at S307, S636/639, and S1101 were not significantly affected in ERα^{LivKO} hepatocytes (FIG. 4L). Palmitate treatment stimulated IRS1 phosphorylation at S302 and reduced IRS1 protein levels and insulin-induced AKT phosphorylation. ERα overexpression improved palmitate-induced insulin resistance, indicated by a decrease in pIRS1-S302 and increases in IRS1, pAKT-S473, and pAKT-T308 protein levels (FIG. 4M). Taken together, these results indicate that ERα interacts with IRS1 and increases IRS1 protein levels, thereby stimulating insulin sensitivity.

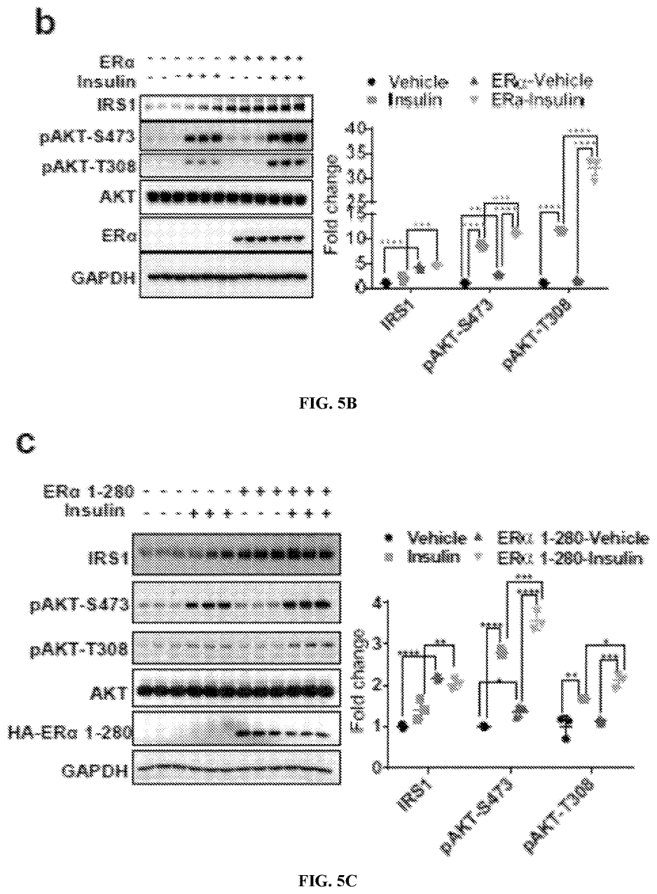

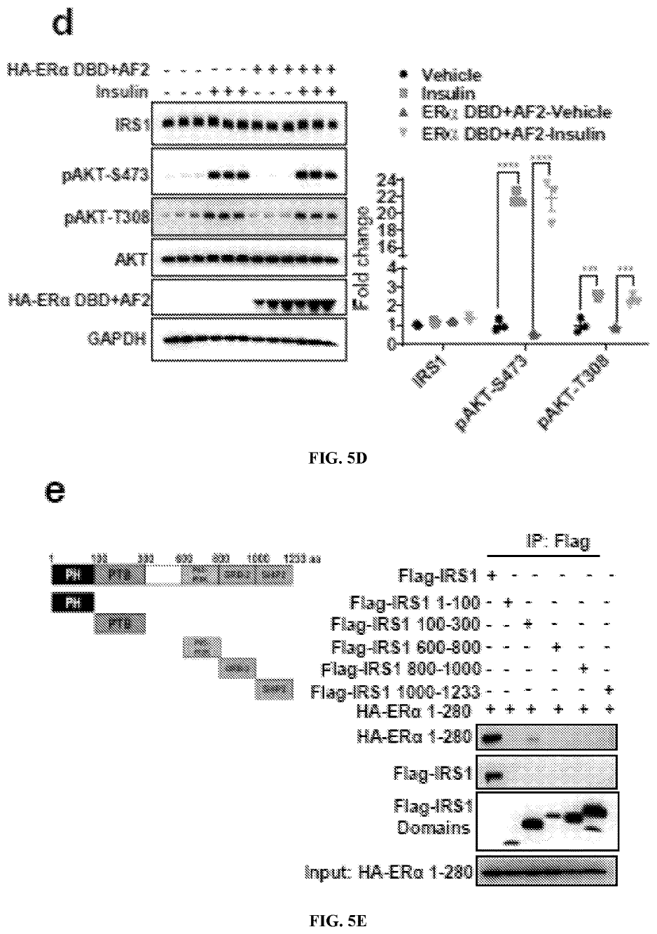

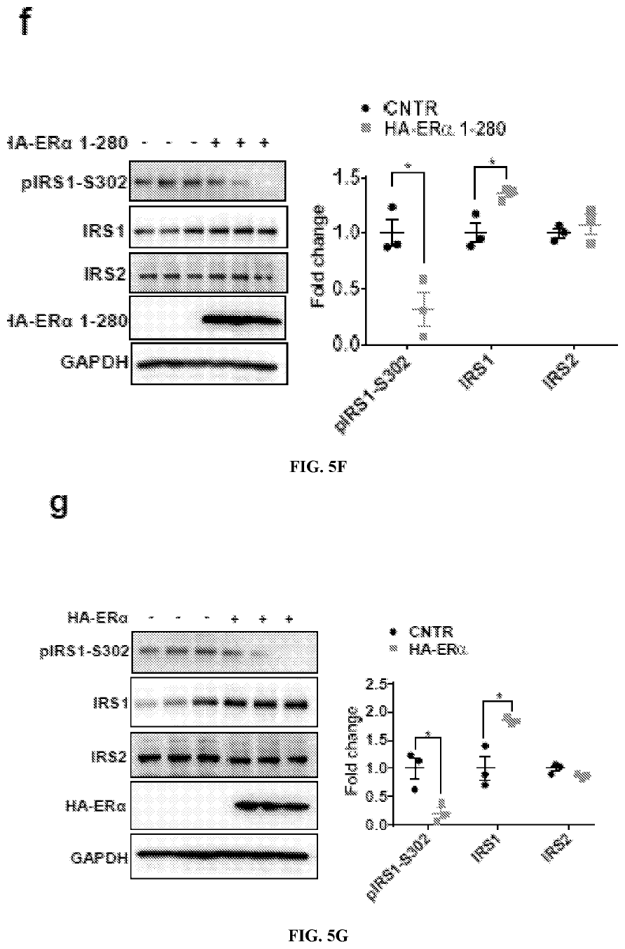

ERα-1-280 domain increases insulin sensitivity through enhancing IRS1 protein stability. ERα is composed of different function domains, including AF (activation function) 1, DBD (DNA binding domain), and AF2. To further investigate how ERα enhances IRS1 protein stability, different ERα domain plasmids were generated and their effects on hepatic insulin sensitivity were tested (FIG. 5A). Consistent with the results in primary hepatocytes, ERα overexpression significantly increased insulin sensitivity in HepG2 cells, indicated by significant increases in IRS1 protein levels and insulin-induced AKT phosphorylation (p<0.0001; FIG. 5B). ERα AF1+DBD (1-280) overexpression significantly increased IRS1 protein levels by 43% (p<0.01) and insulin-induced AKT phosphorylation by 26% (p<0.05) in HepG2 cells (FIG. 5C). However, overexpression of ERα DBD+AF2 did not significantly stimulate insulin sensitivity (FIG. 5D). In line with ERα, ERα 1-280 showed interaction with the IRS1 100-300 domain (FIG. 5E). Gain-of-function of ERα 1-280 decreased pIRS1-S302 by 62% (p<0.05) and increased IRS1 protein abundance by 36% (p<0.05), but had no effect on IRS2 protein abundance in HepG2 cells (FIG. 5F). Consistently, HepG2 cells with ERα overexpression showed a 79% decrease in pIRS1-S302 (p<0.05), 85% increase in IRS1 protein levels (p<0.05), and no significant effect on IRS2 levels (FIG. 5G). The ubiquitination of IRS1 but not IRS2 was attenuated by ERα 1-280 (FIGS. 5H and 5I).

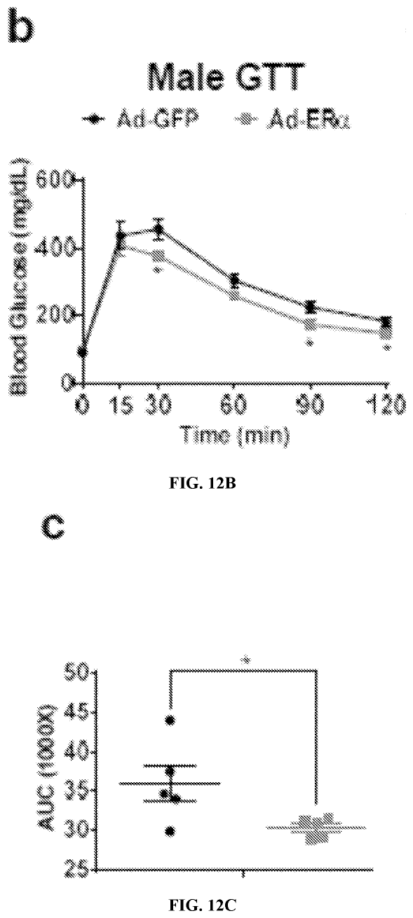

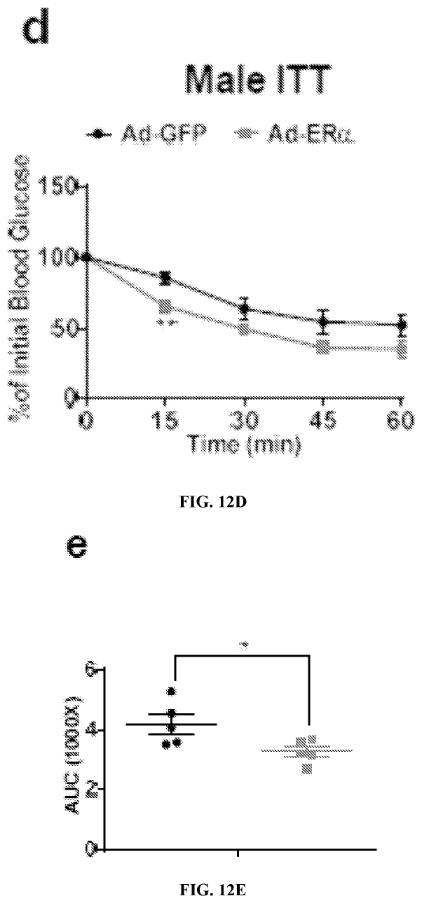

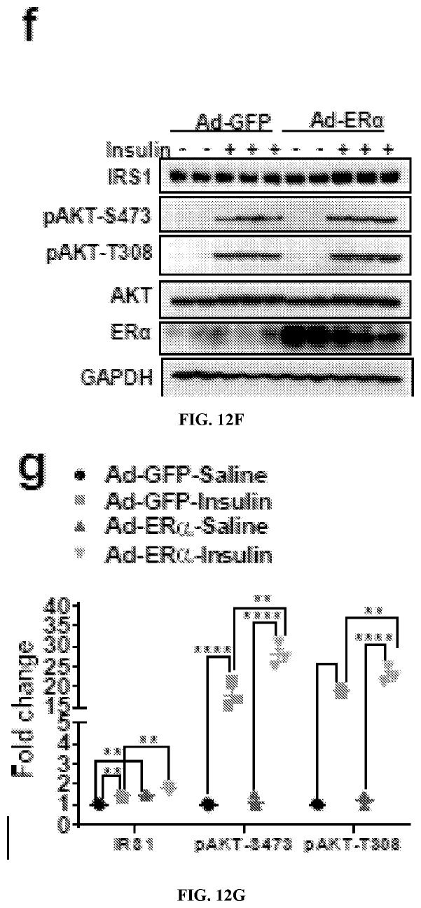

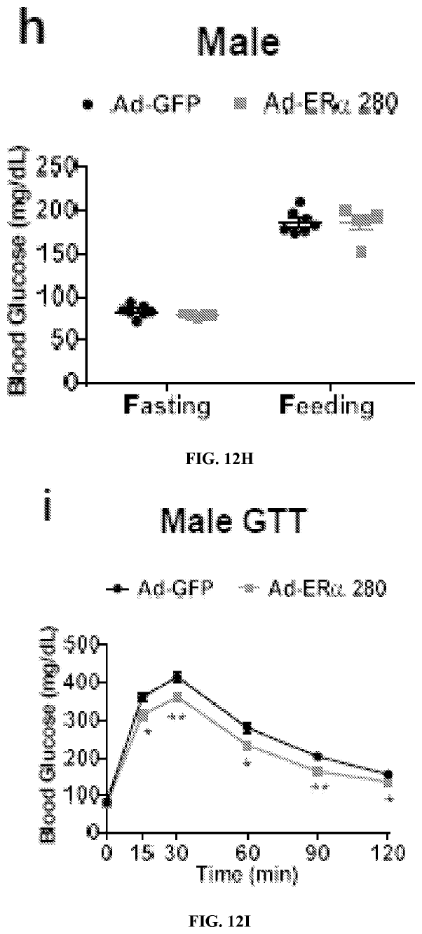

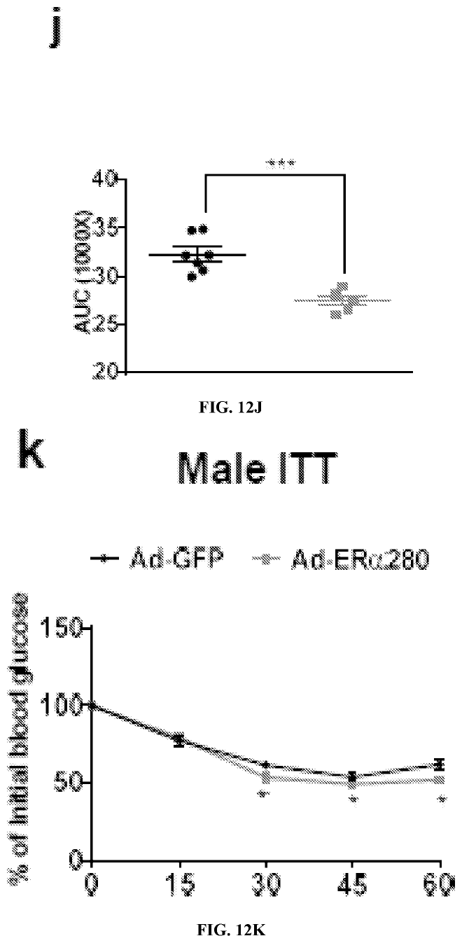

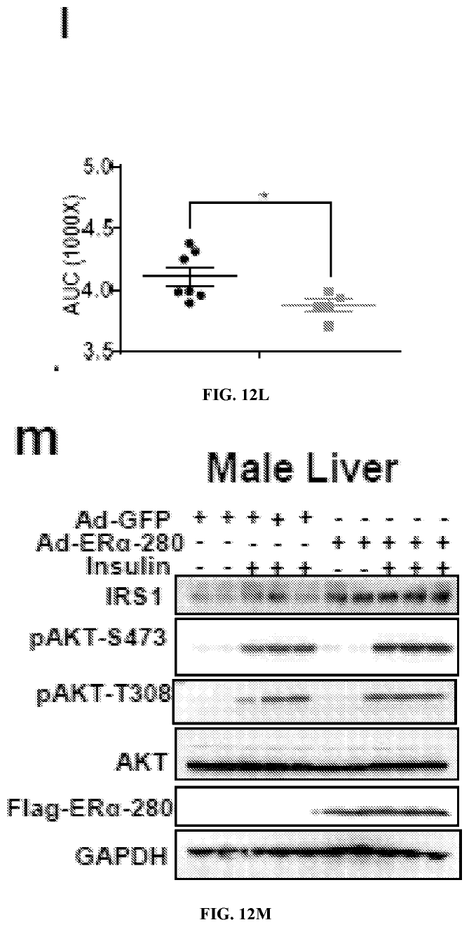

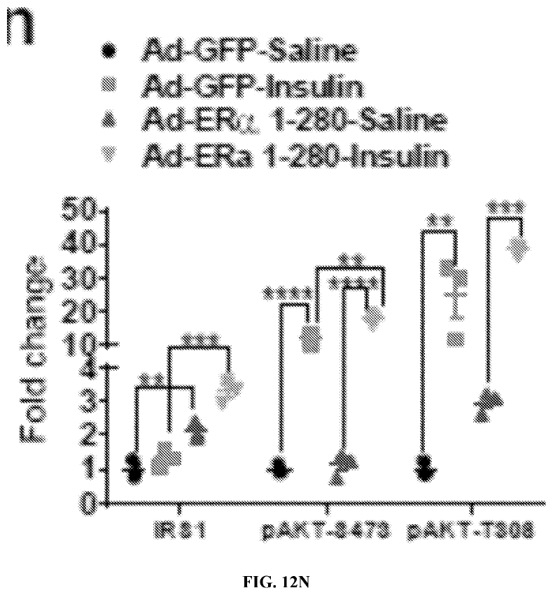

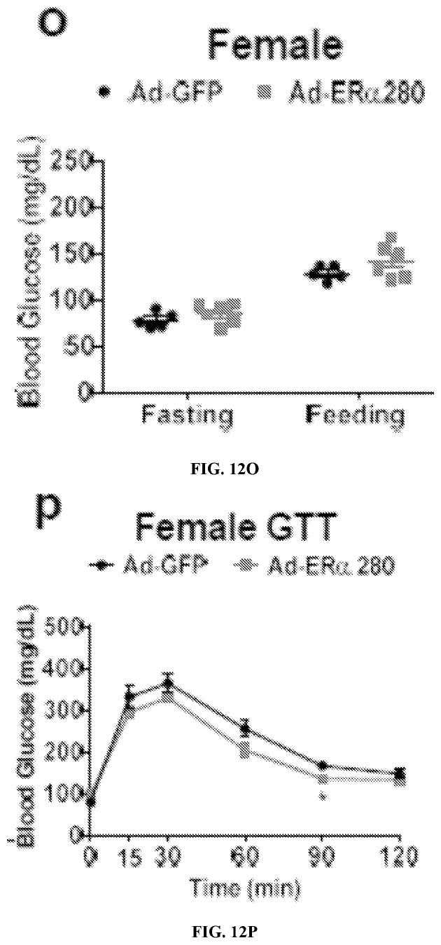

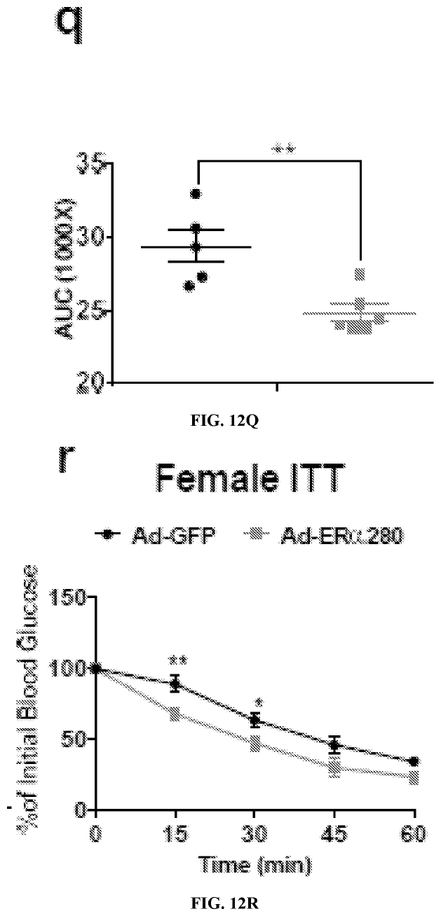

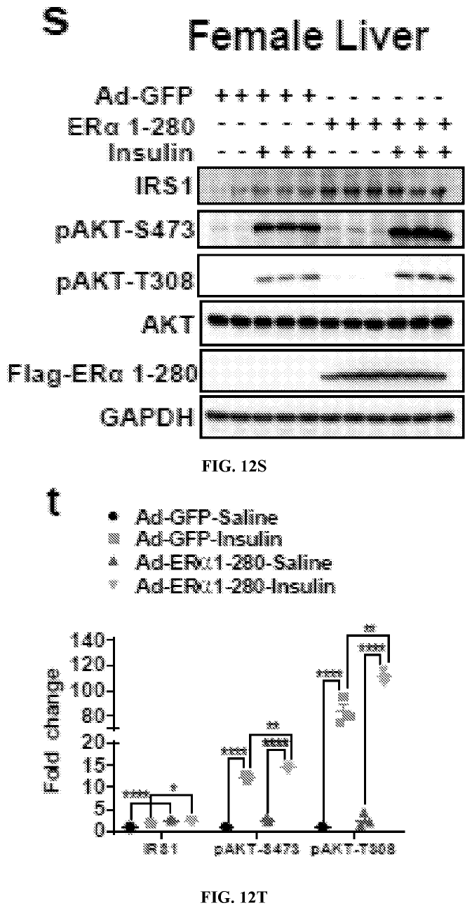

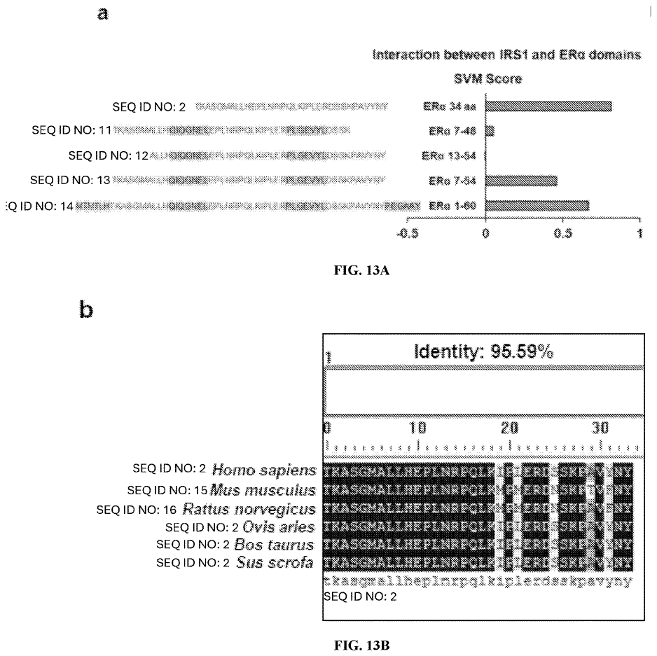

The role of ERα and ERα 1-280 in the regulation of insulin sensitivity in WT mice was detected. Adenovirus (Ad)-mediated ERα overexpression led to a 15% decrease in random feeding blood glucose (Ad-GFP: 196.0±10.5 vs. Ad-ERα: 167.5±4.3 mg/dL; p<0.05) in WT male mice (FIG. 12A). In mice with ERα overexpression, glucose tolerance and insulin sensitivity were significantly improved by 16% and 22%, respectively (p<0.05, FIGS. 12B, 12C, 12D, and 12E). Moreover, hepatic insulin sensitivity was increased by ERα gain-of-function, indicated by increases in IRS1 protein levels by 46% (p<0.01), insulin-induced pAKT-S473 by 55%, and pAKT-T308 by 22% (p<0.01, FIGS. 12F and 12G). Ad-ERα 1-280 was also injected into WT mice and ERα 1-280 overexpression had no significant effect on the blood glucose in both random feeding and overnight fasting male WT mice (FIG. 12H). During the glucose and insulin challenge, glucose disposal rate was significantly increased in ERα 1-280 overexpressed male WT mice (FIGS. 12I, 12J, 12K, and 12L). Accordingly, ERα 1-280 gain-of-function increased IRS1 abundance by 110% and enhanced insulin-induced AKT phosphorylation by ~50% in the liver of male WT mice (p<0.01, FIGS. 12M and 12N). In female WT mice, ERα 1-280 overexpression had no significant effect on the blood glucose under both feeding and fasting conditions (FIG. 12O). Glucose tolerance and insulin sensitivity were significantly improved in female WT mice infected with Ad-ERα 1-280 (FIGS. 12P, 12Q, and 12R). Furthermore, hepatic insulin sensitivity was enhanced by ERα 1-280 overexpression in the liver of female WT mice, as evidenced by a 160% increase in IRS1 protein levels (p<0.0001) and ~20-30% increase in insulin-stimulated AKT phosphorylation (p<0.01, FIGS. 12S and 12T). These results show that ER 1-280 is an important domain to mediate ERα-stimulated insulin sensitivity in vivo and in vitro.

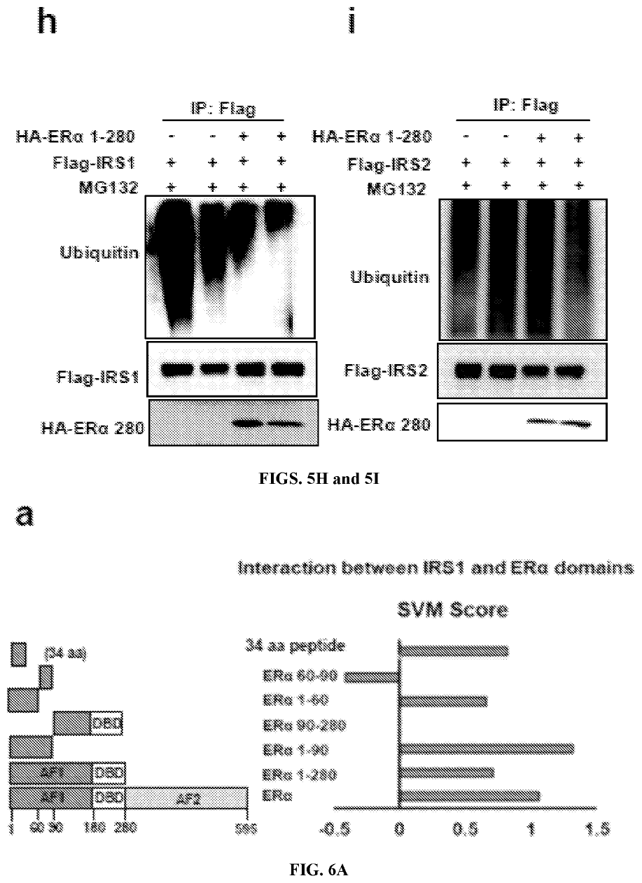

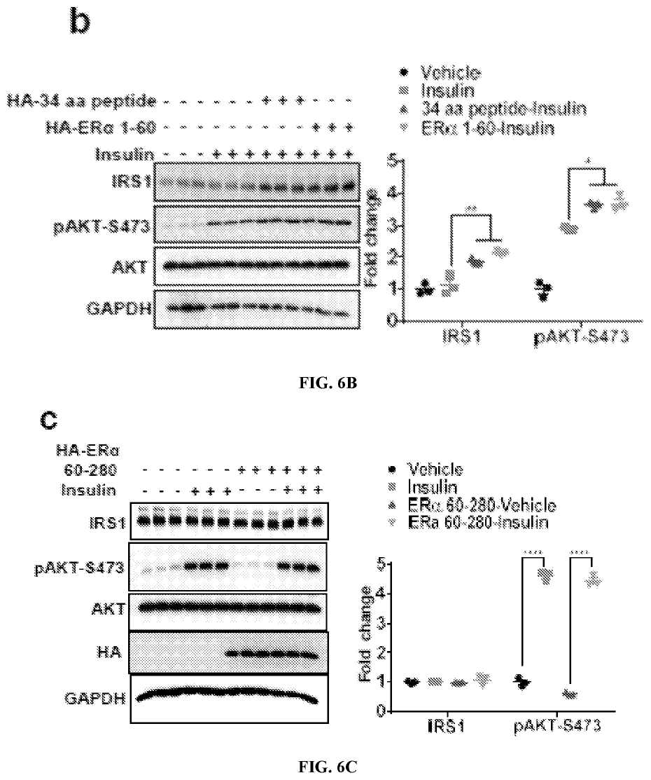

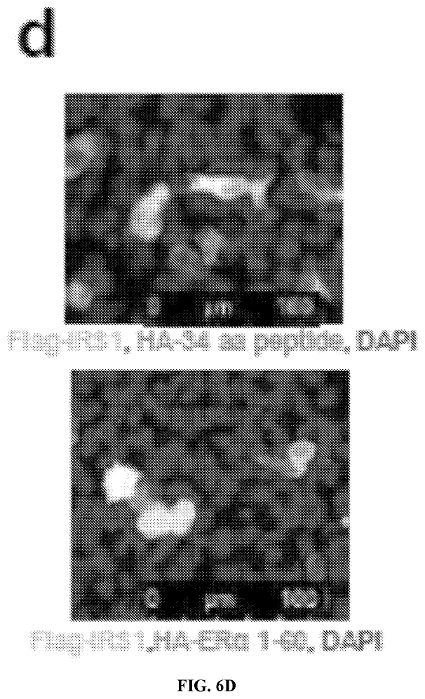

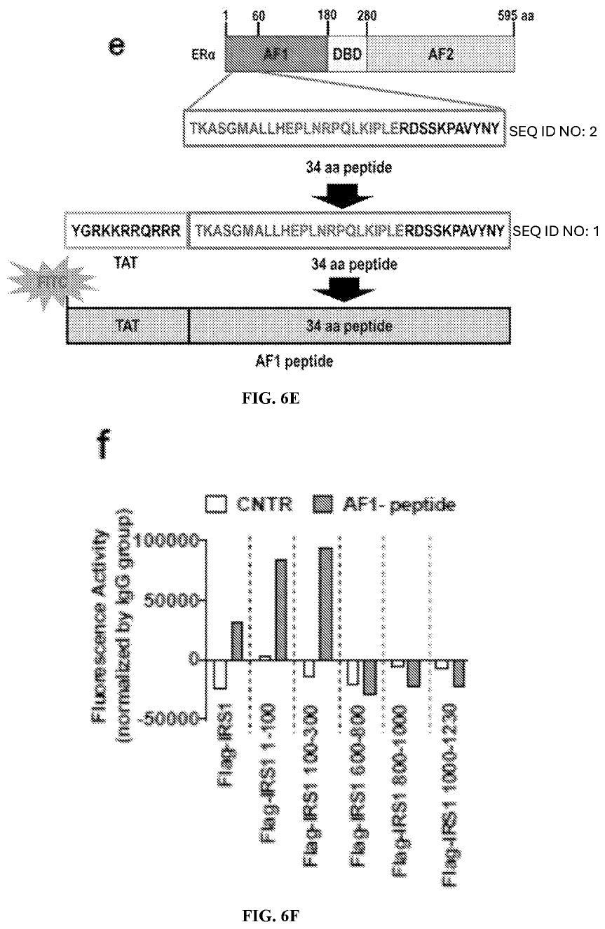

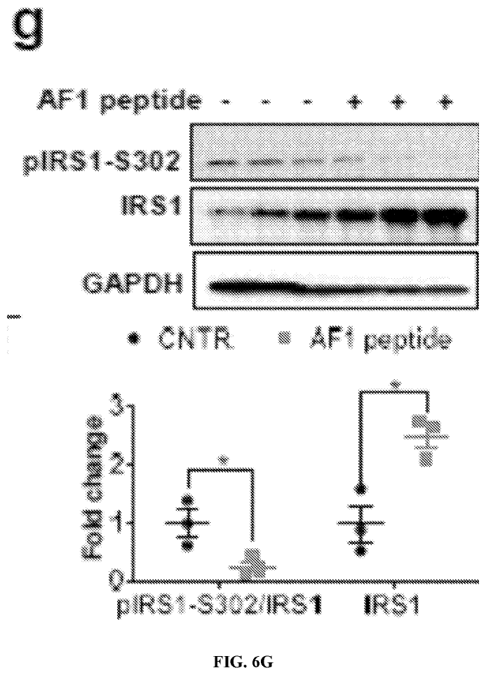

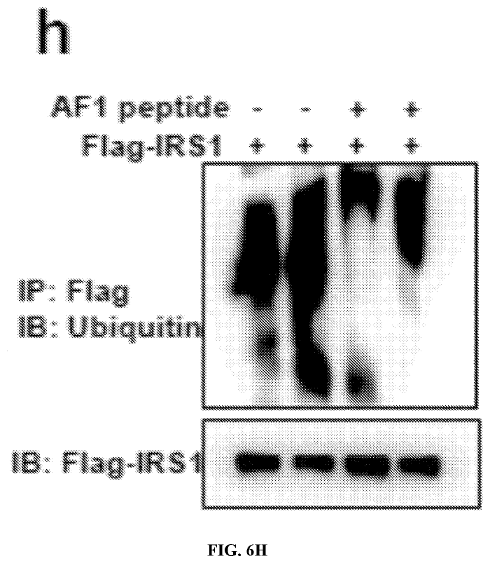

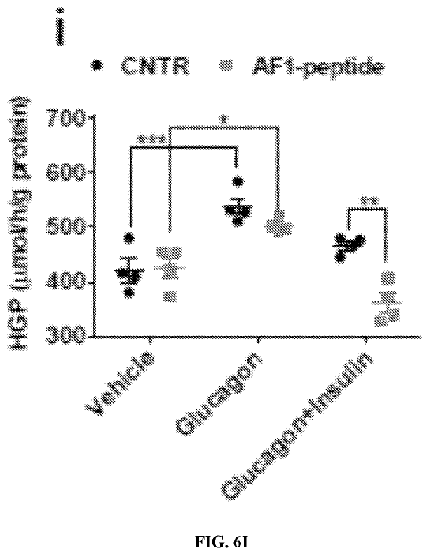

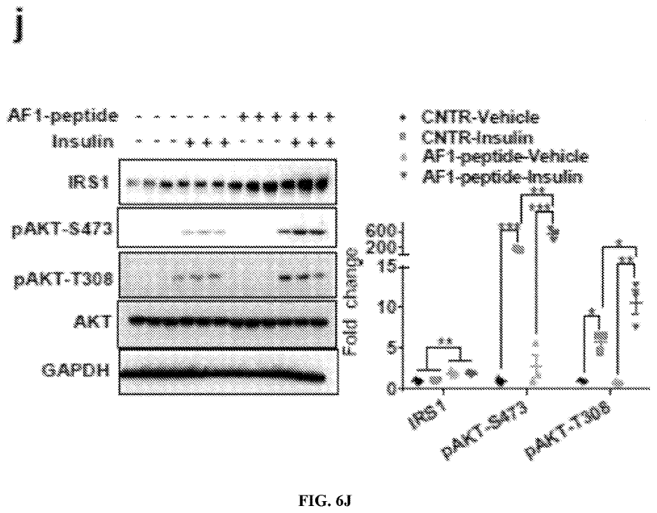

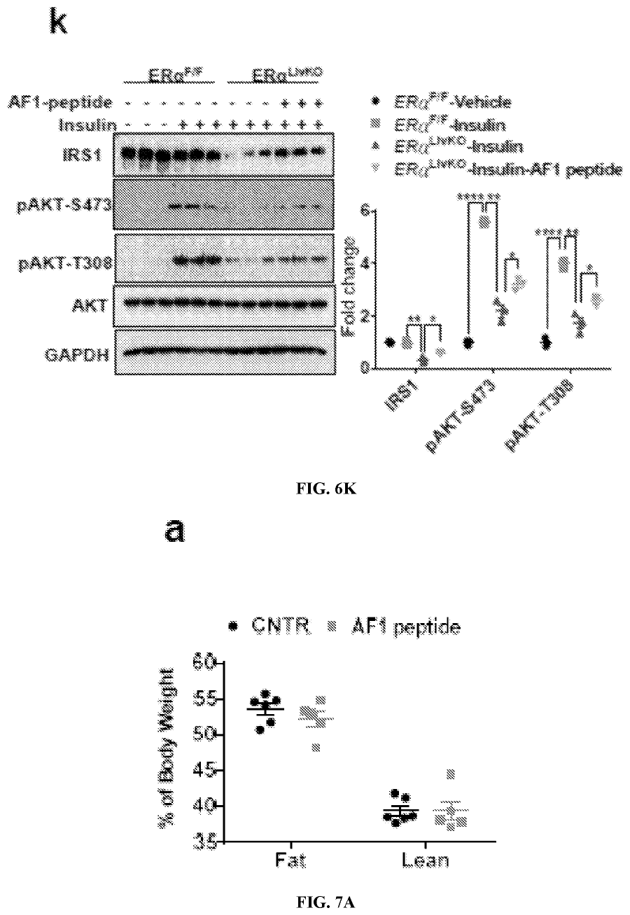

ERα-derived AF1 peptide increases hepatic insulin sensitivity through IRS1. Considering ERα 1-280 is an important domain in the regulation of insulin sensitivity, the amino acid sequence was analyzed with a goal of designing a peptide that functions as an insulin sensitizer. A computational analysis was performed and an interaction score (SVM score) was calculated between ERα domain and IRS1. We set “0” as threshold (SVM score>0 indicates interaction; SVM score<0 indicates no interaction) since it gives a high sensitivity, specificity, and accuracy. The ERα 1-90, but not the 90-280, domain showed a higher interaction score with IRS1, indicating that ERα 1-90 has a higher potential to interact with IRS1. Further analysis of ERα 1-90 domain showed that ERα 1-60 domain and IRS1 had a high interaction score (FIG. 6A). To further shorten the functional peptide, the amino acid of ERα 1-60 domain was also analyzed. The N and C terminals of ERα 1-60 domain (1-6 aa and 55-60 aa) are not essential for its interaction with IRS1, indicated by that ERα 7-54 domain showed a high interaction score with IRS1. However, deletion of ERα 7-12 aa or 49-54 aa impaired its interaction with IRS1, indicated by a low interaction score of ERα 13-54 and 7-48 domains. Finally, the ERα 7-54 domain was narrowed into 34 aa by deleting QIQGENL (SEQ ID NO: 17) and PLGEVYL (SEQ ID NO: 18) sequences, which generates a higher interaction score with IRS1 (FIG. 13A). This ERα-derived 34 aa showed a high similarity (95.59%) among different species (FIG. 13B). To examine the effects of ERα 1-60 and 34-amino acid-peptide on insulin sensitivity, gain-of-function experiments were performed in HepG2 cells. Both overexpression of ERα 1-60 and 34-amino acid-peptide significantly increased IRS1 by ~80% (p<0.01) and enhanced insulin-stimulated AKT phosphorylation by ~26% (p<0.05) in HepG2 cells (FIG. 6B). However, the ERα 60-280 domain had no significant effect on IRS1 protein levels and insulin-induced AKT phosphorylation (FIG. 6C). The ERα 1-60 domain and 34-amino acid-peptide showed colocalization with IRS1, indicating an interaction (FIG. 6D). To further confirm the effect of 34-amino acid-peptide, this peptide was synthesized with a FITC-labelled TAT sequence at the N-terminal of the 34-aa-peptide (termed as AF1 peptide; FIG. 6E). The co-immunoprecipitation assay showed that AF1 peptide interacted with the IRS1 1-300 domain (FIG. 6F). AF1 peptide treatment significantly decreased pIRS1-S302 by 75% (p<0.05) and increased IRS1 protein levels by 50% (p<0.05) in WT primary hepatocytes (FIG. 6G). The ubiquitination of IRS1 was attenuated by AF1 peptide (FIG. 6H). AF1 peptide did not impair glucagon-induced hepatic glucose production (HGP) and significantly enhanced insulin-mediated suppression of HGP by 22% under glucagon challenge (p<0.01) in WT primary hepatocytes (FIG. 6I). Consistently, AF1 peptide increased insulin sensitivity in WT primary hepatocytes, indicated by a 90% increase in IRS1 protein levels (p<0.01) and enhancement in insulin-stimulated AKT phosphorylation at S473 by 210% (p<0.01) and T308 by 82% (p<0.05, FIG. 6J). To detect whether AF1 peptide can rescue ERα deficiency-induced hepatic insulin resistance, ERα deficient primary hepatocytes were treated with the AF1 peptide. Insulin sensitivity was significantly enhanced in ERα deficient primary hepatocytes treated with AF1 peptide, indicated by increases in IRS1, pAKT-S473, and pAKT-T308 protein levels by 80%, 46%, and 50%, respectively (P<0.05). Compared to ERα^{F/F} hepatocytes, the AF1 peptide partially rescued ERα deficiency-induced insulin resistance (FIG. 6K). Collectively, these results indicate that ERα-derived AF1 peptide significantly increases hepatic insulin sensitivity through enhancing IRS1 protein stability.

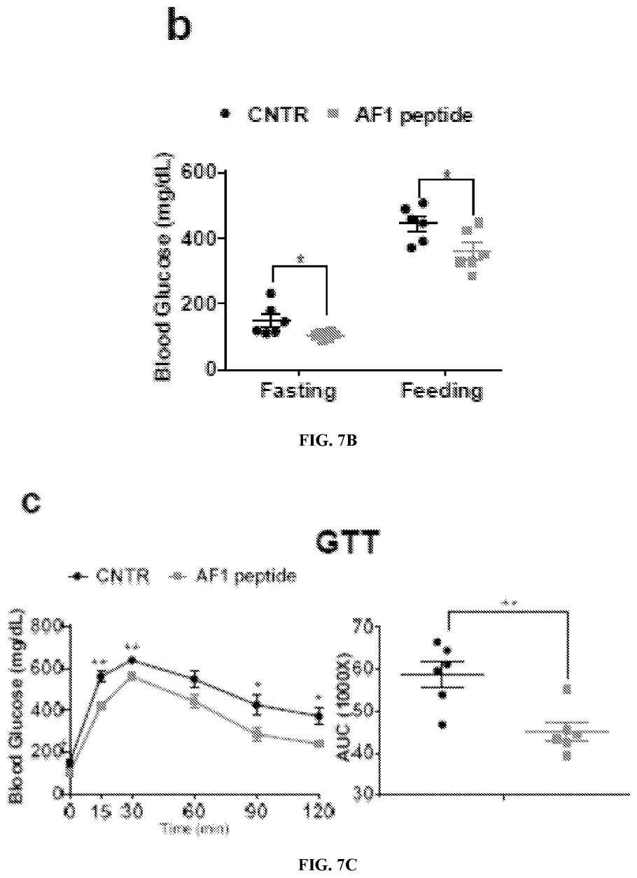

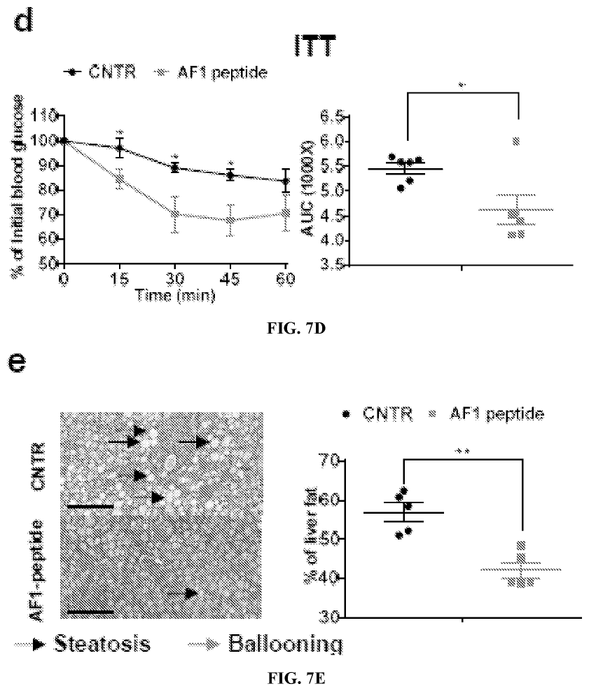

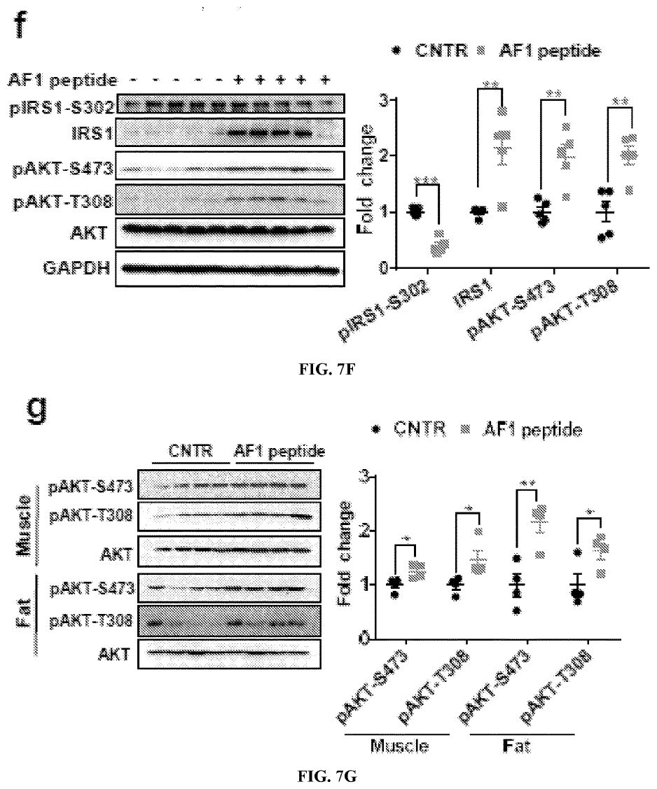

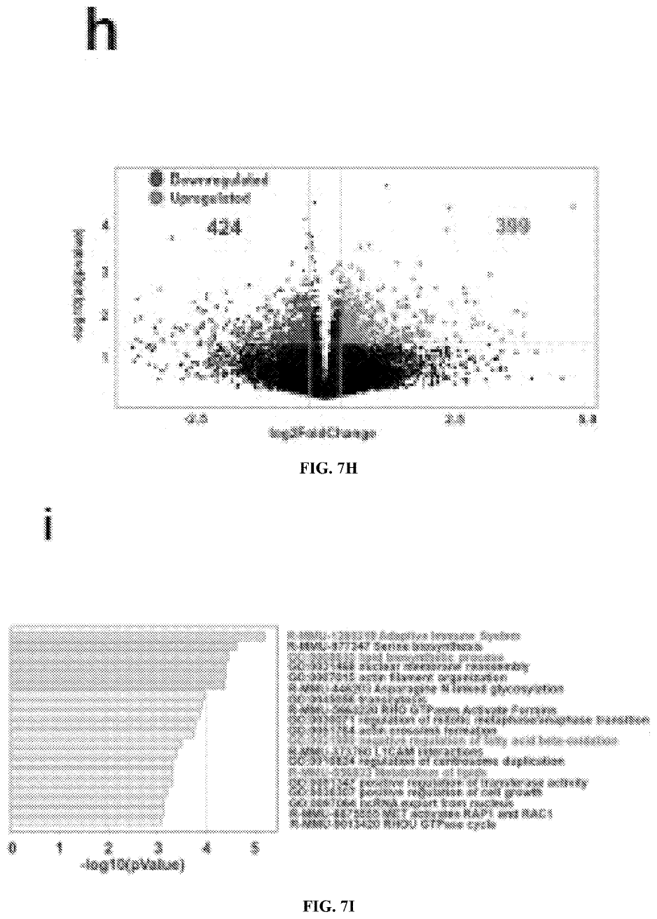

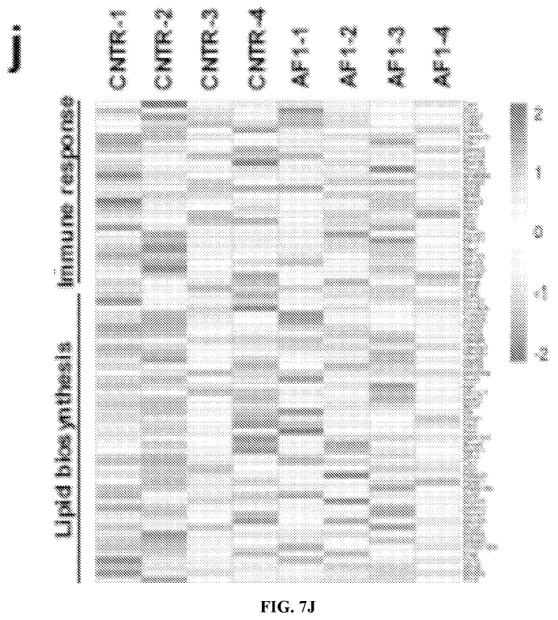

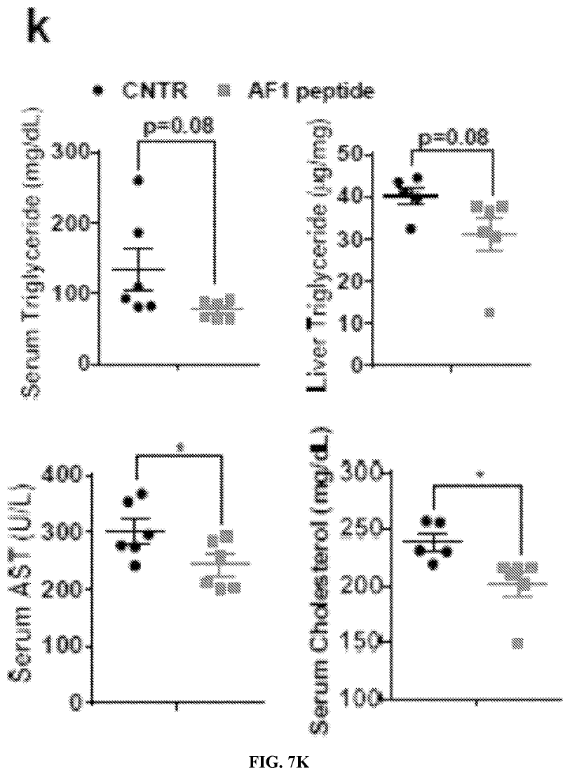

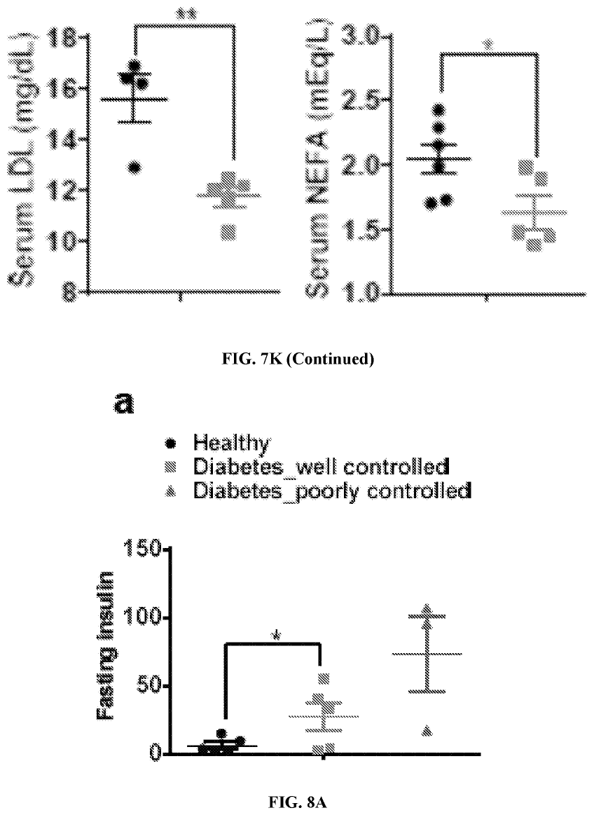

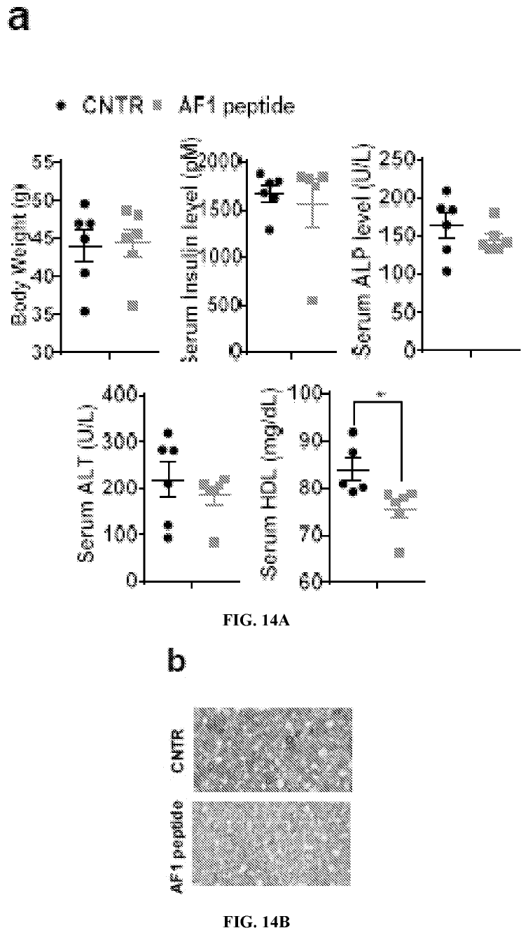

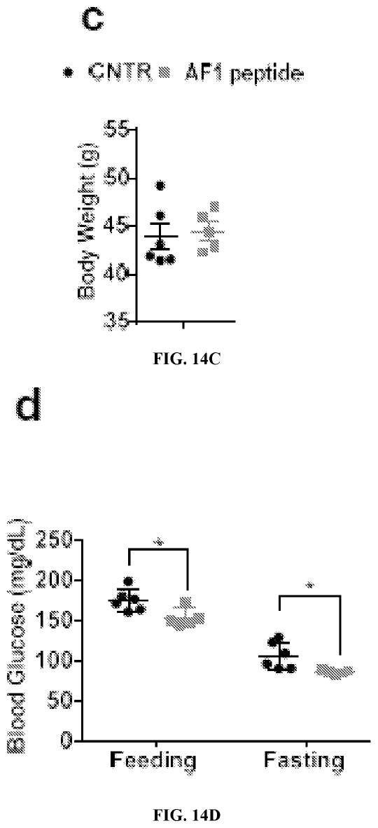

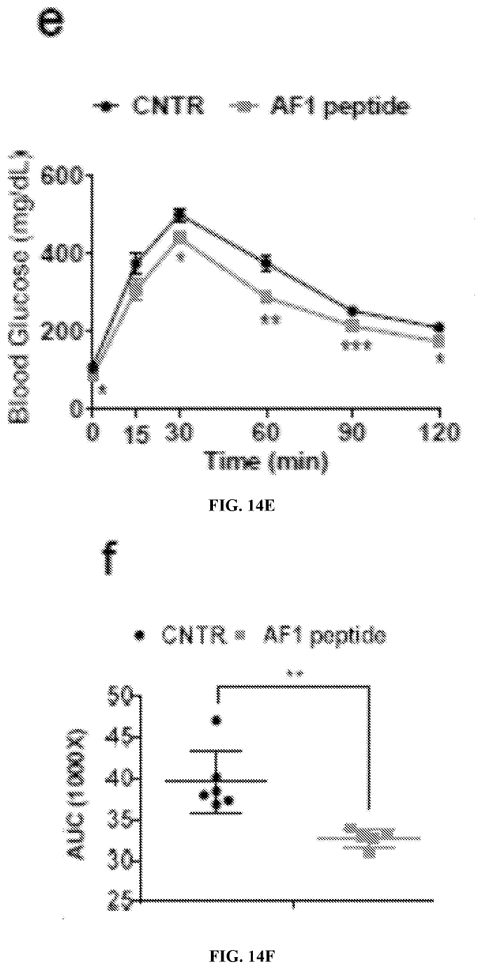

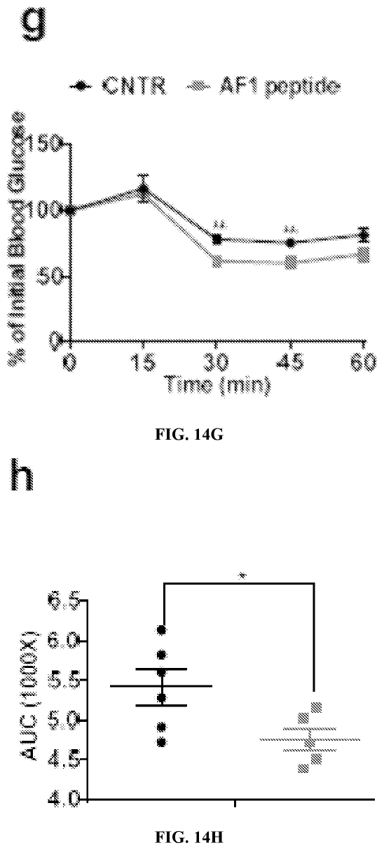

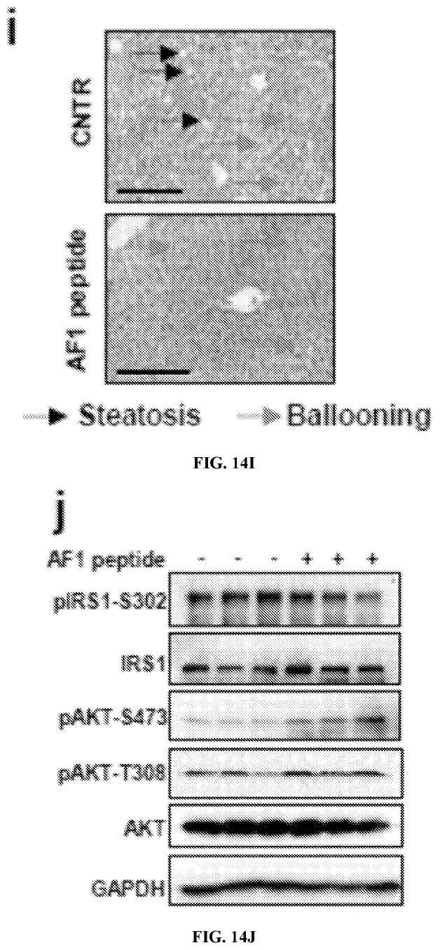

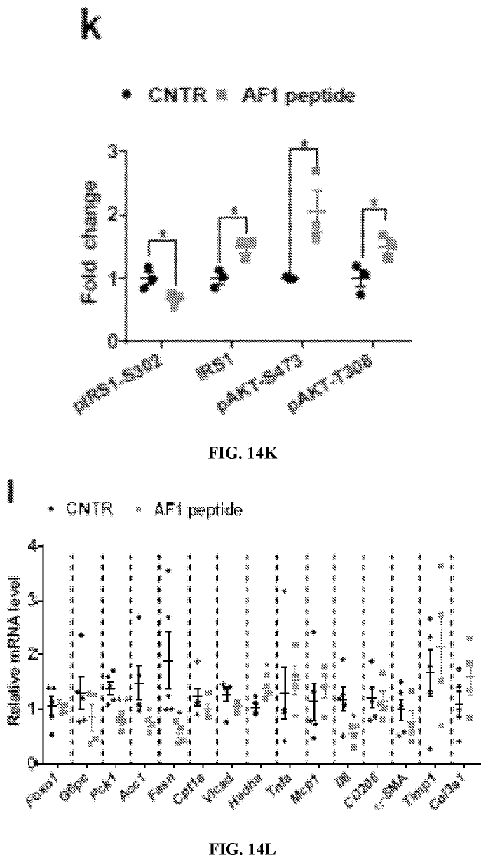

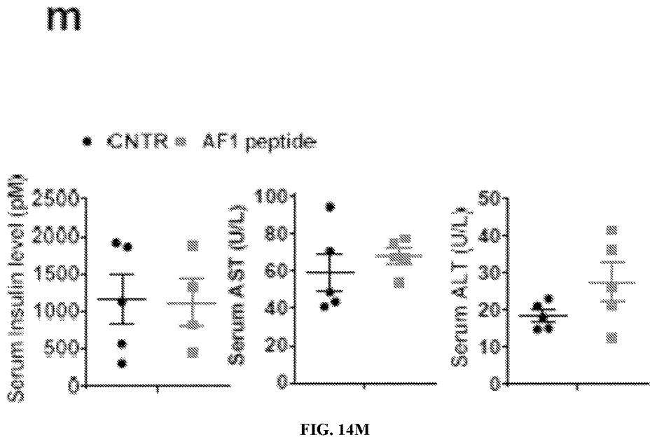

AF1 peptide improves glucose tolerance and insulin sensitivity in diabetic mouse models. To examine the effect of the AF1 peptide on diet-induced glucose dysregulation, AF1 peptide (5 mg/kg body weight, twice per week) was intravenously administered to diet-induced obesity (DIO) or db/db mice for 5 weeks. In db/db mice, AF1 peptide treatment had no significant effect on body weight and body composition (FIG. 7A and FIG. 14A). However, the db/db mice treated with AF1 peptide had a 35% decrease in overnight fasting blood glucose (CNTR: 150.2±19.4 vs. AF1 peptide: 103.8±3.6 mg/dL, p<0.05) and a 22% reduction in random feeding blood glucose (CNTR: 443.7±21.9 vs. AF1 peptide: 360.7±25.1 mg/dL, p<0.05, FIG. 7B). Blood glucose was significantly decreased by AF1 peptide in db/db mice upon glucose or insulin challenge (FIGS. 7C and 7D). Both control and AF1 peptide-treated db/db mice showed hepatocellular ballooning in livers. AF1 peptide significantly decreased liver steatosis, indicated by decreases in liver steatosis structure and liver fat amount (p<0.01; FIG. 7E and FIG. 14B). Hepatic insulin sensitivity was significantly increased by AF1 peptide treatment in db/db mice, indicated by a 61% decrease in pIRS1-S302 (p<0.001) as well as increases in IRS1 by 114%, pAKT-S473 by 98%, and pAKT-T308 by 101% (p<0.01, FIG. 7F). In both skeletal muscle and epididymal fat tissues, pAKT-S473 and pAKT-T308 were significantly increased by AF1 peptide treatment in db/db mice (FIG. 7G). Liver RNA-Seq analysis showed that 399 genes were upregulated and 424 genes downregulated by AF1 peptide treatment in db/db mice (FIG. 7H). Pathway analysis of differentially expressed genes showed that immune response, lipid biosynthesis, and lipid metabolism pathways were significantly attenuated by AF1 peptide treatment (FIGS. 7I and 7J). AF1 peptide treated db/db mice showed a decreasing trend in both serum and liver triglycerides. Serum AST, cholesterol, LDL, HDL, and NEFA levels were significantly reduced in AF1 peptide treated db/db mice. AF1 peptide had a limited effect on serum insulin, ALP, and ALT levels (FIG. 7K and FIG. 14A).

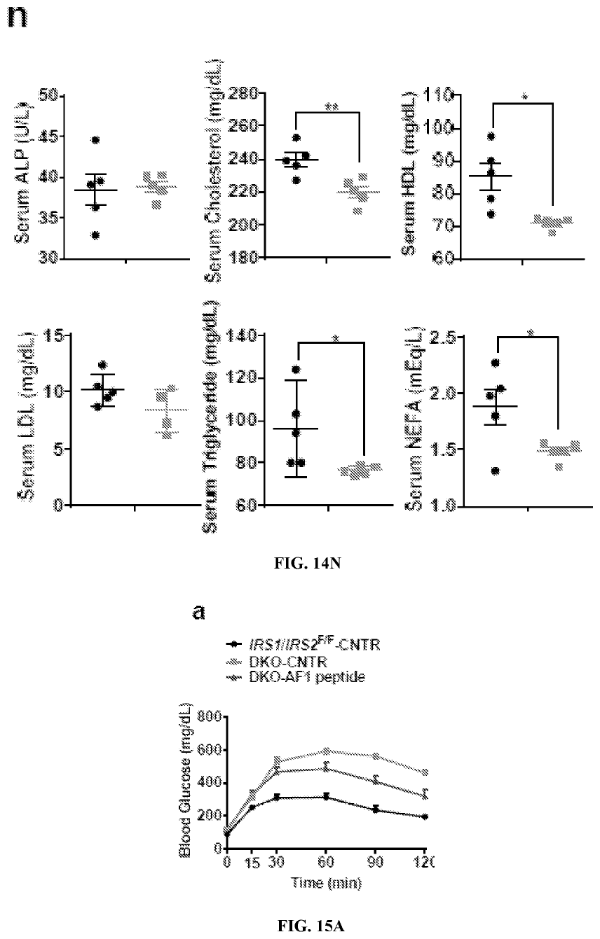

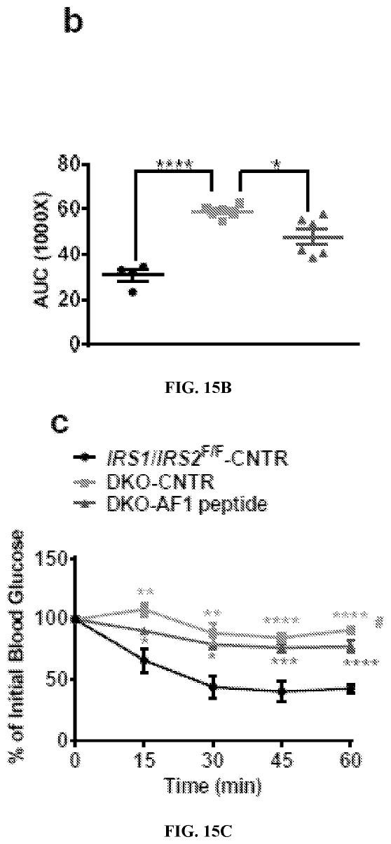

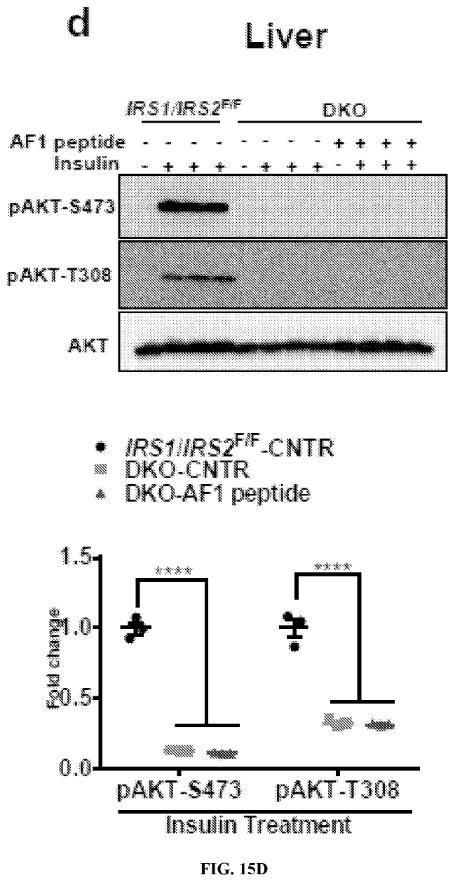

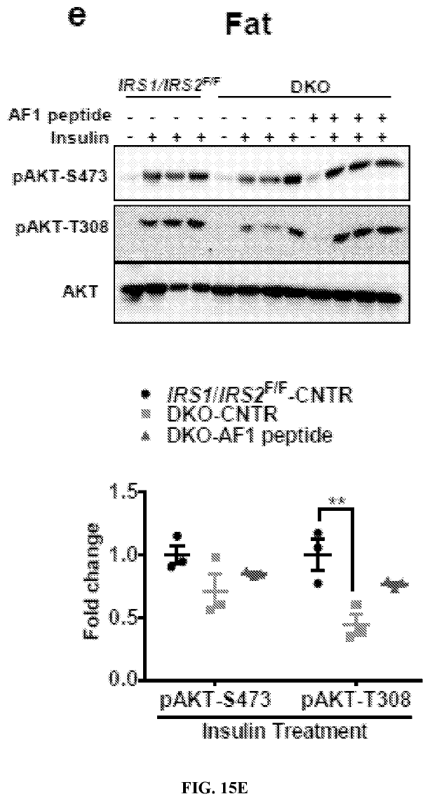

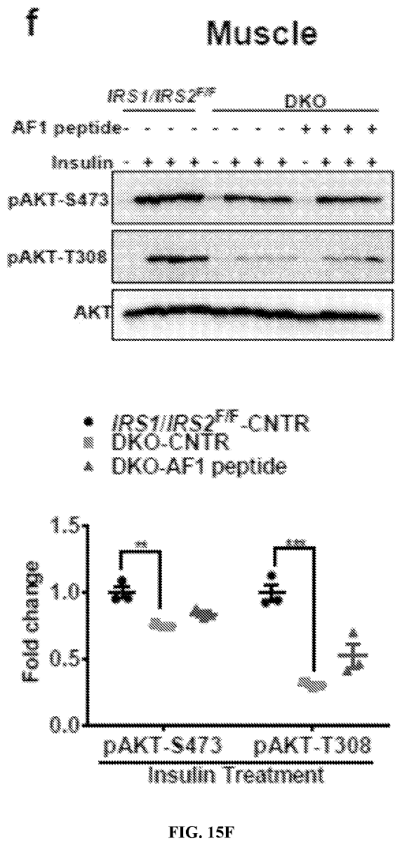

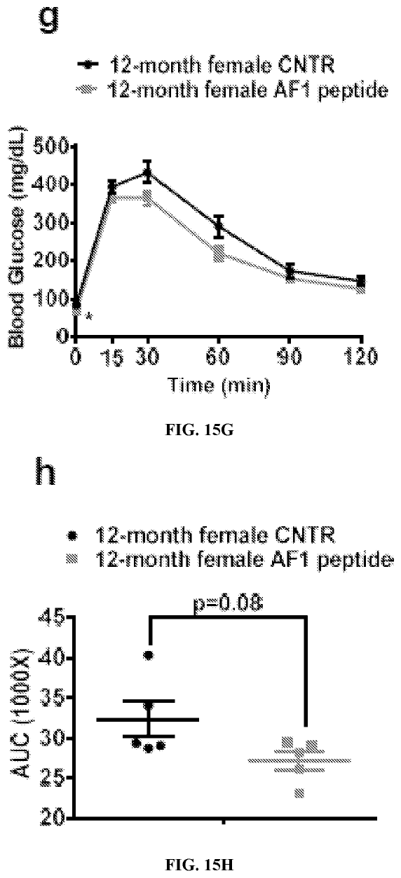

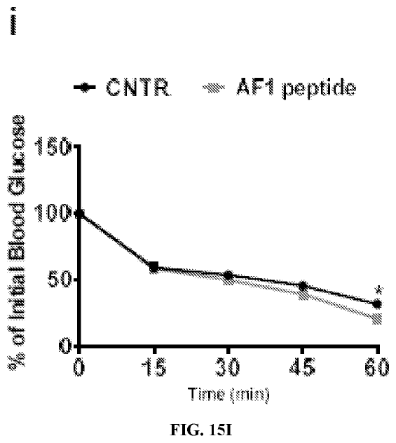

In DIO mice, AF1 peptide treatment did not change body weight (FIG. 14C). However, AF1 peptide treated DIO mice had significant decreases in random feeding blood glucose by 12% (CNTR: 175.5±5.3 vs. AF1 peptide: 154.2±4.7 mg/dL, p<0.05) and overnight fasting blood glucose by 20% (CNTR: 106.2±6.9 vs. AF1 peptide: 85.4±0.2 mg/mL, p<0.05, FIG. 14D). Consistently, glucose tolerance and insulin sensitivity were significantly improved by AF1 peptide in DIO mice (FIGS. 14E, 14F, 14G, and 14H). In the liver of DIO mice, AF1 peptide ameliorated fat accumulation (FIG. 14I) and significantly decreased pIRS1-302 by 32% (p<0.05) and increased IRS1, pAKT-S473, and pAKT-308 by 49%, 100%, and 51%, respectively (p<0.05, FIGS. 14J ad 14K). Gene expression analysis showed that AF1 peptide treatment significantly decreased mRNA expression of gluconeogenic gene (Pck1), lipogenic gene (Fasn), and inflammatory cytokine (Il6) as well as increased mRNA expression of FAO gene (Hadha) in the liver of DIO mice (FIG. 14L). Serum insulin, AST, ALT, and ALP were not changed by AF1 peptide. Serum lipid profile was significantly improved by AF1 peptide treatment, indicated by decreases in cholesterol by 8%, triglyceride by 21%, and NEFA by 21%. AF1 peptide treated DIO mice also showed decreases in both serum HDL and LDL (FIGS. 14M and 14N). The effect of AF1 peptide was further detected in liver-specific IRS1 and IRS2 knockout (DKO) mice. DKO mice showed severe glucose intolerance and insulin resistance and AF1 peptide treatment significantly improved glucose homeostasis and insulin sensitivity in DKO mice (FIGS. 15A, 15B, and 15C). In the liver, insulin-induced AKT phosphorylation was dramatically diminished in DKO mice, which could not be rescued by AF1 peptide treatment (FIG. 12D). In the skeletal muscle and epididymal white adipose tissue (eWAT), DKO mice showed a decrease in insulin-stimulated AKT phosphorylation, compared to control mice, which is partially improved by AF1 peptide treatment (FIGS. 15E and 15F). These results indicate that AF1 peptide improves glucose homeostasis in diabetic mouse models.

Postmenopause in old women is highly associated with increased risk of diabetes. To further examine whether AF1 peptide improves glucose homeostasis in aged females, 12-month-old female mice were treated with 5 mg/kg body weight AF1 peptide via intravenous injection (twice per week) and the AF1 peptide led to a nonsignificant increase in glucose tolerance and modest improvement in insulin sensitivity (FIGS. 15G, 15H, and 15I). These results indicate that AF1 peptide may improve glucose homeostasis in aging females, especially after menopause.

### DISCUSSION

ERα is the major isoform of estrogen receptor in the regulation of energy balance and glucose homeostasis, which is mediated by a non-genomic pathway. It was previously shown that ERα increased insulin sensitivity by activating the AKT-FOXO1 pathway in response to E2. Herein, ERα enhanced insulin action in a ligand-independent manner. Mechanistically, ERα bound to IRS1 and increased its stability through inhibiting IRS1-S302 phosphorylation and ubiquitin-induced degradation. ERα 1-280 domain played an important role in ERα-stimulated insulin sensitivity. Finally, based on the feature of ERα 1-280 domain amino acid sequence, a peptide was designed that acts as an insulin sensitizer, increasing insulin sensitivity and improving glucose homeostasis in obese mice.

The incidence of diabetes in men is much higher than that in women. After menopause, women gradually develop obesity and insulin resistance and are at high risk of type 2 diabetes, but hormone replacement therapy improves insulin sensitivity and glycemic control. These results show that E₂ signaling pathway plays an important role in the regulation of insulin sensitivity and glucose homeostasis. ERα plays a dominant role in mediating the effect of E₂ on the regulation of energy balance and glucose homeostasis. Consistently, hepatic ERα, but not ERβ, deficiency impaired glucose tolerance and insulin sensitivity. E2-ERα signaling exerts its function through both genomic and non-genomic mechanisms. A previous study showed that recovery of ERα non-genomic function rescues ERα deficiency-induced dysregulation of energy metabolism and glucose homeostasis (Park C J, et al. Genetic rescue of nonclassical ERα signaling normalizes energy balance in obese Era-null mutant mice. The Journal of clinical investigation 121, 604-612 (2011)), suggesting that ERα non-genomic pathway mainly mediates its role in metabolic regulation. E₂-ERα signaling stimulates PI3K activity, thereby increasing insulin sensitivity and suppressing HGP. Herein, the present disclosure provides evidence that E₂-ERα activates insulin signaling pathway independent of IRS1 and IRS2. Compared to control mice, DKO female mice showed significantly impaired glucose tolerance and insulin sensitivity upon glucose and insulin challenge. However, glucose handling and insulin sensitivity in DKO female mice were significantly improved, as compared to DKO male mice; such effect is abolished in TKO female mice. Therefore, in addition to the classic insulin signaling pathway (insulin receptor-IRS-PI3K-AKT), E₂-ERα-PI3K-AKT signaling pathway enhances insulin sensitivity independent of IRS. Moreover, hepatic ERα deletion impaired insulin sensitivity and glucose homeostasis in both male and OVX female mice, indicating that ERα also regulates glucose metabolism in a ligand-independent manner. In DKO male mice, hepatic ERα knockout did not further aggravate glucose intolerance and insulin resistance, which suggests that IRS1 and 2 are required for ERα ligand-independent effect. Therefore, ERα controls insulin sensitivity in two major non-genomic pathways: the ligand-dependent E₂-ERα-PI3K-AKT pathway and the ligand-independent ERα-IRS-PI3K-AKT pathway. Estrogen-ERα signaling plays an important role in control of energy homeostasis. In HFD-fed female but not male mice, hepatic ERα deletion led to a significant increase in body weight and fat mass, which can be attributed to estrogen-mediated ERα signaling pathway.

IRS1 and IRS2 are required for insulin-induced carbohydrate and lipid metabolism. A previous study reported that ERα regulated endogenous IRS1 and IRS2 degradation in breast cancer cells (Morelli C, Garofalo C, Bartucci M, Surmacz E. Estrogen receptor-α regulates the degradation of insulin receptor substrates 1 and 2 in breast cancer cells. Oncogene 22, 4007-4016 (2003)), suggesting that IRS proteins may be the target of ERα in regulating glucose homeostasis and insulin sensitivity in mice. Herein, it was found that ERα interacted with both IRS1 and IRS2 but only regulated IRS1 protein abundance, with a limited effect on IRS2 protein levels in mouse primary hepatocytes. The mRNA expression levels of Irs1 and Irs2 were not affected in ERα deficient hepatocytes, showing that ERα controls IRS expression through a post-translational modification. ERα protected against ubiquitination-mediated IRS1 degradation but not IRS2. IRS1 and IRS2 contain a highly similar amino-terminal pleskstrin homology (PH) and phosphostyrosin-binding domain (PTB), whereas the tail region of IRS1 and IRS2 are poorly conserved. A previous study showed that E3 ubiquitin ligase MG53 promoted IRS1 ubiquitination and degradation but had no effect on IRS2 (Song R, et al. Central role of E3 ubiquitin ligase MG53 in insulin resistance and metabolic disorders. Nature 494, 375-379 (2013)). Therefore, the present disclosure found that distinct effects of ERα on IRS1 and IRS2 protein expression can be attributed to the differential regulation of E3 ubiquitin ligase in IRS1 and IRS2. This data showed that E₂ treatment had no effect on the interaction between ERα and IRS1, showing that ERα interacts with IRS1 in a ligand-independent manner. Considering E₂ stimulates ERα nuclear translocation and IRS1 exists in both cytosol and nucleus, it is possible that ERα interacts with IRS1 in both cytosol and nucleus.

Multiple serine phosphorylation sites of IRS1 have been indicated to be correlated to insulin resistance, including S302, S307, S636/639, and S1101. Herein, the present disclosure shows that ERα interacted with the IRS1 PTB (100-300 amino acids) and inhibited IRS1-S302 phosphorylation, which prevents IRS1 degradation. In diabetic mouse liver, IRS1-S302 phosphorylation was significantly increased and suppression of IRS1-302 phosphorylation blocked JNK1-induced insulin resistance. IRS1-S302 phosphorylation, mainly stimulated by mTOR/S6K pathway, impaired its binding to insulin receptor and disrupted insulin signaling. Alanine mutations of IRS1 at S302, S307, and S612 protected against fat-induced insulin resistance in the mouse skeletal muscle. These results indicate that IRS1-302 phosphorylation plays an important role in the development of T2DM. However, a previous study showed that IRS1-S302A knock-in mice had normal glucose homeostasis and muscle insulin sensitivity under physiological condition (Copps K D, Hançer N J, Qiu W, White M F. Serine 302 phosphorylation of mouse insulin receptor substrate 1 (IRS1) is dispensable for normal insulin signaling and feedback regulation by hepatic S6 kinase. Journal of Biological Chemistry 291, 8602-8617 (2016)). Considering the role of IRS1-S302 phosphorylation in insulin resistance, the phenotype of IRS1-S302A knock-in mice under pathological condition should be evaluated. Herein, it was observed that ER enhanced IRS1 protein abundance through inhibiting IRS1 ubiquitination, which is potentially mediated by IRS1-S302 phosphorylation.

Hepatic ERα expression levels were decreased in both mice and humans with diabetes. Interestingly, hepatic ERα expression levels were only significantly decreased in poorly controlled diabetic patients, which can be attributed to hyperinsulinemia-induced reprogramming of liver transcriptomics. These results indicate that hepatic ERα may be a key checkpoint for the progression of diabetes. ERα consists of the N-terminal activation function-1 (AF1) domain, the DNA binding domain (DBD), and the C-terminal activation function-2 (AF2) domain. The AF1 domain is constitutively active, whereas the AF2 domain is located within the hormone binding domain and is more ligand-regulatable. Both the AF1 and AF2 domains contribute to the transcriptional activity of target genes. The AF2 domain, but not AF1 domain, is required by estrogen-mediated prevention of obesity and insulin resistance. Selective activation of the AF1 domain by tamoxifen protects mice from diet-induced obesity, steatosis, and insulin resistance; this result shows that AF1 domain plays an important role in the regulation of metabolic homeostasis in a ligand-dependent manner. Herein, an AF1 domain was identified that mediated the ligand-independent effect of ERα on insulin sensitivity. Deletion of ERα from the liver resulted in hepatic insulin resistance in male, female, and OVX female mice. Overexpression of ERα in the liver of male mice increased hepatic insulin sensitivity, showing that ERα regulates insulin sensitivity in a ligand-independent manner. The individual ERα domains, including AF1, DBD, and AF2, had no significant effect on hepatic insulin sensitivity (Data not shown). However, the AF1-DBD domain, rather than DBD-AF2 domain, copied the effect of ERα on hepatic insulin action in vitro and in vivo. Although the AF1 domain alone showed no effect on insulin sensitivity, it is required to enhance insulin signaling. Recombinant AF1 domains can be made to fold by conjunction with the DBD domain, thereby affecting AF1 domain function and promoting its interaction with other factors in a ligand-independent manner. The conjunction of AF1 with the DBD domain changes AF1 function state and renders its interaction with IRS1, thus increasing IRS1 protein stability. Therefore, the ligand-independent effect of ERα on insulin sensitivity is mainly mediated through the AF1 domain.

Peptides have gained an increased interest in pharmaceutical research due to their high specificity, efficiency, and safety. A number of peptides have been designed to prevent the development of obesity, including glucagon-like peptide-1 (GLP-1), natriuretic peptide, and defensin-derived peptide. Herein, an insulin sensitizing peptide was designed based on the interaction between IRS1 and ERα. The N-terminal of AF1-DBD domain (1-60 aa) exhibited a high interaction score with IRS1 and enhanced insulin sensitivity. The AF1-DBD 1-60 domain was narrowed down and a 34-aa-peptide was screened out as a insulin sensitizer that interacts with IRS1 and increases insulin sensitivity. HIV TAT-derived peptide, a small basic peptide, can deliver target proteins into living cells. Thus, the conjunction of TAT-derived peptide to the N-terminal of 34-aa-peptide (AF1 peptide) was prepared. These results showed that the AF1 peptide interacted with the IRS1 1-300 domain, increased IRS1 protein stability, and enhanced insulin sensitivity. However, the AF1 peptide did not completely normalize ERα deficiency-induced insulin resistance in primary hepatocytes, showing that ERα also mediates hepatic insulin sensitivity through other mechanisms. In the obese mouse model, the AF1 peptide improved glucose tolerance, insulin sensitivity, and serum lipid profiles without affecting body weight. Although the AF1 peptide increased IRS1 protein abundance in the obese mouse liver, other targets regulated by AF1 peptide to mediate its beneficial effects on glucose and lipid homeostasis cannot be ruled out. It has been shown that ERα regulated glucose homeostasis and insulin sensitivity in skeletal muscle and adipose tissue. In skeletal muscle, global ERα knockout significantly decreased insulin-mediated glucose uptake in female mice. Adipose tissue ERα deletion resulted in enlarged adipocytes as well as increased adipose tissue inflammation and fibrosis in both male and female mice. Glucose tolerance and insulin sensitivity were largely impaired in the adipose tissue knockout male mice. Indeed, AF1 peptide significantly increased insulin sensitivity in both skeletal muscle and adipose tissue (eWAT) of db/db mice. In the DKO mouse model, the AF1 peptide had no effect on hepatic insulin sensitivity, indicating that the effect AF1 peptide requires IRS proteins. However, glucose homeostasis was still improved in AF1 peptide treated DKO mice, which is partially attributed to the improvement of insulin sensitivity in adipose tissue and skeletal muscle. These results indicate that AF1 peptide targets adipose tissue and skeletal muscle, thereby regulating insulin sensitivity and glucose homeostasis. Moreover, AF1 peptide improved insulin sensitivity and glucose homeostasis in aging female mice (12-month-old); this result shows that AF1 peptide improves menopause-induced dysregulation of glucose homeostasis. Overall, the AF1 peptide is an effective insulin sensitizer to treat T2DM. Collectively, ERα enhanced insulin sensitivity in a ligand-independent manner. Mechanistically, ERα interacted with IRS1 (100-300 domain), thereby increasing IRS1 protein stability. Based on ERα function, an AF1 peptide was designed that acts as an insulin sensitizer to improve glucose homeostasis and lipid profile in diabetic mouse models.

### Methods

Animal studies. All animal experiments were performed following procedures approved by the Texas A&M University Institutional Animal Care and Use Committee. Mice are housed at 22-24° C. and maintained on a 12 h light/12 h dark cycle with a standard chow diet (58% calories from carbohydrate, 18% from fat, and 24% from protein, 3.1 kcal/g, with a reduced phytoestrogen content; Teklad Diet) ad libitum. Liver-specific ERα (ERα^{LivKO}) or ERβ (ERβ^{LivKO}) knockout mice were generated by crossing ERα or ERβ flox mice (gift from Dr. Yong Xu, Baylor College of Medicine) with Albumin-Cre mice purchased from The Jackson Laboratory (Strain #003574), respectively. Liver IRS1 and IRS2 double knockout (DKO) mice were generated by breeding IRS1 and IRS2 flox (IRS1^{L/L}::IRS2^{L/L}) mice with Albumin-Cre mice. Liver IRS1, IRS2, and ERα triple knockout (TKO) mice were generated by crossing DKO mice with ERα^{LivKO} mice. All mice were on C57B/6J background. Db/db mice were purchased from The Jackson Laboratory (Strain #000697). Female mice at age of 2-month-old underwent a bilateral ovariectomy (OVX) surgery; these mice were used to perform experiments 4 weeks after OVX surgery. All the mice experiments were performed with age- and gender-matched mice with their health status examined daily by the investigators and technicians from Texas A&M University LARR Animal Facility. Littermate controls were used if possible.

Obese mouse models and AF1 peptide treatment. For the high-fat diet (HFD)-induced obese mouse model, the 6-8-week-old male mice were fed with HFD (HFD; 60% kcal from fat, D12492; Research diet, New Brunswick, NJ) for 11 weeks. Then mice received 5 mg/kg body weight control or AF1 peptide through retro orbital injection (twice per week; 5 weeks). For genetic obesity mouse model, the 8-week-old db/db mice were administered with 5 mg/kg body weight control or AF1 peptide through retro orbital injection (twice per week; 5 weeks). Glucose tolerance and insulin tolerance tests were performed in the fourth and fifth week after treatment, respectively. Body composition was measured using EchoMRI™ one day before sacrificing the mice.

Ovariectomy and estrogen replacement. Female mice were randomly assigned to experimental groups. Bilateral ovariectomy surgery was performed in female mice. Placebo or 17β-estradiol (E2) pellet (0.05 mg/pellet, 60-day release; Innovative Research of America, Sarasota, FL) was subcutaneously implanted into ovariectomized female mice. Female mice were subjected to E₂ or placebo pellet implantation at same time of bilateral ovariectomy surgery.

Primary hepatocytes isolation and cell culture. Primary hepatocytes were isolated. Male mice at age of 8-12 weeks were infused with a perfusion buffer A (calcium and magnesium-free HBSS supplemented with 10 mM HEPES, 50 mM EGTA, 1 mM glucose, and 1% penicillin-streptomycin, pH 7.4) through portal vein for 6-8 min. Then perfusion buffer A was changed to buffer B (calcium and magnesium-free HBSS supplemented with 10 mM HEPES, 1 mM CaCl₂), 1 mM glucose, 1% penicillin-streptomycin, and 0.5 mg/mL collagenase II, pH 7.4). When showed signs of cracking at the surface, the liver was transferred into the ice-cold serum-free DMEM. Liver cells were suspended and passed through a 70 μm cell strainer, followed by centrifuge at 1700 rpm for 2 min. Pellets were resuspended with Percoll at final concentration of 36% and centrifuged at 1800 rpm for 6 min. The hepatocyte pellet was washed and suspended with DMEM supplemented with 10% FBS and 1% penicillin-streptomycin in collagen-coated plates. The HepG2 cell line was purchased from Abcam and cultured with DMEM medium supplemented with 10% FBS and 1% penicillin-streptomycin in collagen-coated plates. The human embryo kidney (HEK) 293 cell line was purchased from Sigma and cultured with DMEM medium supplemented with 10% FBS and 1% penicillin-streptomycin. FBS-free DMEM medium was used to culture cells during the experiments.

Glucose and insulin tolerance tests. For glucose tolerance tests, mice were fasted for 16 h and intraperitoneally (i.p.) injected with dextrose (1 g/kg body weight for mice fed with regular diet; 0.5 g/kg body weight for db/db mice or mice fed with HFD). For insulin tolerance tests, mice were fasted for 6 h and i.p. injected with insulin solution (1 U/kg body weight for male mice; 0.75 U/kg body weight for female mice). Blood glucose levels were measured using glucometer at indicated time points.

Hepatic glucose production. The primary mouse hepatocytes were isolated from 8-12-week-old male mice. Cells were resuspended in DMEM medium (2% FBS and 1% penicillin-streptomycin) for 3 h and cultured in HGP buffer (118 mM NaCl, 2.5 mM CaCl2, 4.8 mM KCl, 25 mM NaHCO₃, 1.1 mM KH2PO4, 1.2 mM MgSO4, 10 mM ZnSO4, 0.6% BSA, 10 mM HEPES, 10 mM sodium DL-lactate, and 5 mM pyruvate, pH 7.4). Then cells were pretreated with 50 nM glucagon for 30 min, followed by 10 nM insulin treatment for 3 h, and glucose level in the medium was measured using Amplex Red Glucose Assay. The results were normalized by cell protein abundance.

RNA extraction and quantitative real-time PCR. Total RNA was extracted from livers or primary hepatocytes using TRIzol (Invitrogen, Thermo Fisher Scientific), purified with chloroform, 80% ethanol, isopropanol, and RNA column, eluted with RNase free water, and reverse transcribed into cDNA using iScript Reverse Transcription Supermix (Bio-Rad, Hercules, CA). Quantitative real-time PCR (qPCR) was conducted using using SsoAdvanced Universal SYBR Green Supermix (Bio-Rad, Hercules, CA) on CFX384 real-time PCR detection system (Bio-Rad, Hercules, CA). Q-PCR started with 95° C. incubation for 3 min, followed by 39 cycles of 95° C. for 5 s and 60° C. for 30 s. The threshold cycle (CT) values were obtained and used to calculate the relative transcriptional level of target genes by 2^{-ΔΔCT} method with Cyclophilin (Cyp) as an internal control. Statistical analysis was performed using 2^{-λΔCT}.

Immunofluorescence staining. Cells were fixed with 4% formaldehyde solution, followed by incubation with blocking buffer (1×PBS/5% normal serum/0.3% Triton X-100) for 1 h. After rinsing three times using PBS, cells were incubated with primary antibodies overnight at 4° C. Cells were washed with PBS and incubated with secondary antibodies (anti-mouse Alexa Flour 594 Conjugated) at room temperature 1-2 h. Cells were covered with DAPI-contained mounting medium. Fluorescent signal was observed using Confocal microscopy (Leica).

Hematoxylin and eosin staining and Oil Red O staining. Livers were embedded into paraffin after being fixed by PFA, and then cut into 5 μm sections. After deparaffinization and hydration, liver sections were stained with hematoxylin for 5 min. After differentiation by 1% acid alcohol, liver sections were stained with 1% eosin for 3 min. Finally, liver sections were dehydrated and mounted with mounting media. For Oil Red O staining, liver frozen sections were prepared and fixed in 10% neutral buffered formalin for 10 min after air dry for 1 h. Liver sections were stained in Oil Red O working solution for 15 min after a quick dip in 60% isopropanol. Liver sections were then stained with hematoxylin for 3 min and covered with aqueous mounting gel. Liver sections were observed using slide scanner (Leica).

Adenovirus infection. For ERα and ERα 1-280 overexpression mouse model, 8-12-week-old male mice were administered with adenovirus-HA-ERα and HA-ERα 1-280 (1×10¹⁰ genome copies/mouse) through retro-orbital injection. Two weeks after adenovirus injection, glucose and insulin tolerance tests were performed. For ERα overexpression in primary hepatocytes, 100 MOI adenovirus ERα was used to inject primary mouse hepatocytes for 24 h, followed by 100 nM insulin treatment for 1 h. Cell proteins were collected using lysis buffer for Western blot analysis.

Co-immunoprecipitation and Western blotting analysis. Flag-labelled IRS or truncated IRS proteins were co-transfected into HEEK293 cells with HA-labelled ERα and its truncated proteins for 30 h using lipofectamine 3000 (Thermo Fisher), followed by 10 μM MG132 treatment for 1 h. Cell proteins were extracted using TNE buffer and incubated with primary antibody (Anti-Flag Rabbit) overnight at 4° C. Flag-labelled proteins were immunoprecipitated using Dynabeads Protein G. HA-labelled proteins were detected using Western blotting. For Western blotting, tissues or cells were homogenized in a RIPA buffer supplemented with protease and phosphatase inhibitors. Equal amount of proteins were analyzed using Wester blotting assays. The proteins were separated with Sodium dodecyl-sulfate polyacrylamide gel electrophoresis (SDS-PAGE) system. The gel was transferred onto a 0.45 μm PVDF membrane at 100 V for 2 h at 4° C., followed by blocking in 5% BSA for 1 h at room temperature. The membrane was then incubated with primary antibodies at 4° C. overnight, followed by secondary antibody incubation for 1 h at room temperature. Protein bands were detected using a ChemiDoc image system. Protein signals were quantified using ImageJ 1.53K (National Institutes of Health, Bethesda, Maryland).

Protein interaction prediction. Interaction between IRS1 protein and ERα protein domains was predicted using ProPrInt (URL: webs.iiitd.edu.in/raghava/proprint/submit).

AF1 peptide and IRS1 protein interaction. HEK293 cells were transfected with Flag-IRS1 or Flag-IRS1 truncated domains using lipofectamine 3000 for 30 h, followed by treatment of 10 μM control and AF1 peptides labelled with FITC fluorescence for 3 h. After 10 μM MG132 treatment for 1 h, cell protein was extracted, and Flag-labelled proteins were immunoprecipitated. The intensity of control and AF1 peptide was calculated through measuring FITC fluorescence signal with multiplex reader. The results were normalized using IgG group.

Serum lipid profile analysis. Serum triglyceride, AST, ALT, cholesterol, LDL, HDL, NEFA, and ALP were measured using DxC 700 AU Chemistry Analyzer (Beckman Coulter).

Serum insulin and liver triglyceride analysis. Serum insulin and triglyceride levels were measured by using mouse insulin ELISA kit (Mercodia) and triglyceride assay kit (Abcam), respectively.

RNA-Seq analysis. Total RNA was isolated from frozen liver tissues of db/db mice treated with control and AF1 peptide (n=4 per group). The RNA-Seq was performed at TIGSS molecular genomics core in Texas A&M University. Analysis of RNA-seq data was performed at Texas A&M University high performance research computing institute. Briefly, the STAR aligner program was employed to align RNA sequence reads into mouse genome using STAR. HTseq was used to count the sequences that can be mapped to gene features. DEseq2 was used to normalize the raw read counts and process differential expression gene analysis. The significantly expressed genes were determined by p value less than 0.05. Function analysis of differentially expressed genes was performed using Metascape.

Human liver gene expression analysis. The human liver gene profile dataset was downloaded from NCBI (GSE15653). The differential gene expression was analyzed using GEO2R. Pearson correlation coefficient between liver ERα gene expression level and fasting blood glucose, HbA1C, or HOMA-IR was calculated.

Statistical analysis. Results were shown as mean±SEM, with n representing the number of biological replicates. GraphPad Prism 6.01 was used for statistical analysis and graphs. No samples or data were excluded from the study for statistical purposes. Each in vitro experiment was independently performed twice or three times to ensure reproducibility. Animals were randomly assigned into control and treatment groups in all studies. The correlation coefficient and its significance between two independent variables were evaluated by Person's correlation test. To calculate p value in the studies with two groups, unpaired two-tailed Student's t tests were performed for statistical analysis. To determine the different significance among multiple groups, one-way ANOVA or two-way ANOVA with Tukey's multiple comparisons test was used. p<0.05 was considered to be statistically significant.

Data availability. New generated data, as well as source data and codes have been deposited in Figshare. Raw counts for bulk RNAseq for livers of control and AF1 peptide treated db/db mice can be downloaded on Figshare under the doi.org/10.6084/m9.figshare.25142975. Publicly available Affymetrix human genome array data from healthy and diabetic individuals were downloaded on the Gene Expression Omnibus under the number GSE15653. Source data are provided with this paper and under the doi.org/10.6084/m9.figshare.25142969. Uncropped blots are available at the doi.org/10.6084/m9.figshare.25142933. Source data are provided with this paper.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

SEQUENCES

1. SEQ ID NO: 1-ER synthetic peptide amino acid sequence
YGRKKRRQRRRTKASGMALLHEPLNRPQLKIPLERDSSKPAVYNY
2. SEQ ID NO: 2-AF1 domain portion sequence
TKASGMALLHEPLNRPQLKIPLERDSSKPAVYNY
3. SEQ ID NO: 3-Cell penetrating peptide amino acid sequence
YGRKKRRQRRR
4. SEQ ID NO: 4-ER synthetic peptide nucleic acid sequence
tatggccgcaaaaaacgccgccagcgccgccgcaccaaagcatctgggatggccctactgcatgagcccctgaaccgtccgcagctcaagat
ccccctggagcgggacagcagcaagcccgccgtgtacaactac
5. SEQ ID NO: 5-AF1 domain portion nucleic acid sequence
accaaagcatctgggatggccctactgcatgagcccctgaaccgtccgcagctcaagatccccctggagcgggacagcagcaagcccgccg
tgtacaactac
6. SEQ ID NO: 6-Cell penetrating peptide nucleic acid sequence
tatggccgcaaaaaacgccgccagcgccgccgc
7. SEQ ID NO: 7-AF1 domain amino acid sequence
MTMTLHTKASGMALLHQIQGNELEPLNRPQLKIPLERPLGEVYLDSSKPAVYNYPEGAAYE
FNAAAAANAQVYGQTGLPYGPGSEAAAFGSNGLGGFPPLNSVSPSPLMLLHPPPQLSPFLQ
PHGQQVPYYLENEPSGYTVREAGPPAFYRPNSDNRRQGGRERLASTNDKGSMAMESAK
8. SEQ ID NO: 8-AF1 domain nucleic acid sequence
atgaccatgaccctccacaccaaagcatctgggatggccctactgcatcagatccaagggaacgagctggagcccctgaaccgtccgcagctc
aagatccccctggagcggcccctgggcgaggtgtacctggacagcagcaagcccgccgtgtacaactaccccgagggcgccgcctacgagt
tcaacgccgcggccgccgccaacgcgcaggtctacggtcagaccggcctcccctacggccccgggtctgaggctgcggcgttcggctccaa
cggcctggggggtttccccccactcaacagcgtgtctccgagcccgctgatgctactgcacccgccgccgcagctgtcgcctttcctgcagccc
cacggccagcaggtgccctactacctggagaacgagcccagcggctacacggtgcgcgaggccggcccgccggcattctacaggccaaatt
cagataatcgacgccagggggcagagaaagattggccagtaccaatgacaagggaagtatggctatggaatctgccaag
9. SEQ ID NO: 9-FULL PROTEIN SEQUENCE FOR THE ER ALPHA PROTEIN
MTMTLHTKASGMALLHQIQGNELEPLNRPQLKIPLERPLGEVYLDSSKPAVYNYPEGAAYE
FNAAAAANAQVYGQTGLPYGPGSEAAAFGSNGLGGFPPLNSVSPSPLMLLHPPPQLSPFLQ
PHGQQVPYYLENEPSGYTVREAGPPAFYRPNSDNRRQGGRERLASTNDKGSMAMESAKET
RYCAVCNDYASGYHYGVWSCEGCKAFFKRSIQGNRHNDYMCPATNQCTIDKNRRKSCQA
CRLRKCYEVGMMKGGIRKDRRGGRMLKHKRQRDDGEGRGEVGSAGDMRAANLWPSPL
MIKRSKKNSLALSLTADQMVSALLDAEPPILYSEYDPTRPFSEASMMGLLTNLADRELVHM
INWAKRVPGFVDLTLHDQVHLLECAWLEILMIGLVWRSMEHPGKLLFAPNLLLDRNQGKC
VEGMVEIFDMLLATSSRFRMMNLQGEEFVCLKSIILLNSGVYTFLSSTLKSLEEKDHIHRVL
DKITDTLIHLMAKAGLTLQQQHQRLAQLLLILSHIRHMSNKGMEHLYSMKCKNVVPLYDL
LLEMLDAHRLHAPTSRGGASVEETDQSHLATAGSTSSHSLQKYYITGEAEGFPATV
10. SEQ ID NO: 10-FULL NUCLEIC ACID SEQUENCE FOR THE ER ALPHA PROTEIN
atgaccatgaccctccacaccaaagcatctgggatggccctactgcatcagatccaagggaacgagctggagcccctgaaccgtccgcagctc
aagatccccctggagcggcccctgggcgaggtgtacctggacagcagcaagcccgccgtgtacaactaccccgagggcgccgcctacgagt
tcaacgccgcggccgccgccaacgcgcaggtctacggtcagaccggcctcccctacggccccgggtctgaggctgcggcgttcggctccaa
cggcctggggggtttccccccactcaacagegtgtctccgagcccgctgatgctactgcacccgccgccgcagctgtcgcctttcctgcagccc
cacggccagcaggtgccctactacctggagaacgagcccagcggctacacggtgcgcgaggccggcccgccggcattctacaggccaaatt
cagataatcgacgccagggtggcagagaaagattggccagtaccaatgacaagggaagtatggctatggaatctgccaaggagactcgctact
gtgcagtgtgcaatgactatgcttcaggctaccattatggagtctggtcctgtgagggctgcaaggccttcttcaagagaagtattcaaggtaa
tagacataacgactatatgtgtccagccaccaaccagtgcaccattgataaaaacaggaggaagagctgccaggcctgccggctccgtaaatgc
tacgaagtgggaatgatgaaaggtgggatacgaaaagaccgaagaggagggagaatgttgaaacacaagcgccagagagatgatggggagg
gcaggggtgaagtggggtctgctggagacatgagagctgccaacctttggccaagcccgctcatgatcaaacgctctaagaagaacagcctg
gccttgtccctgacggccgaccagatggtcagtgccttgttggatgctgagcccccgatactctattccgagtatgatcctaccagacccttca
gtgaagcttcgatgatgggcttactgaccaacctggcagacagggagctggttcacatgatcaactgggcgaagagggtgccaggctttgtgga
tttgaccctccatgatcaggtccaccttctagaatgtgcctggctagagatcctgatgattggtctcgtctggcgctccatggagcacccaggg
aagctactgtttgctcctaacttgctcttggacaggaaccagggaaaatgtgtagagggcatggtggagatcttcgacatgctgctggctacat
catctcggttccgcatgatgaatctgcagggagaggagtttgtgtgcctcaaatctattattttgcttaattctggagtgtacacatttctgtc
cagcaccctgaagtctctggaagagaaggaccatatccaccgagtcctggacaagatcacagacactttgatccacctgatggccaaggcaggc
ctgaccctgcagcagcagcaccagcggctggcccagctcctcctcatcctctcccacatcaggcacatgagtaacaaaggcatggagcatctgt
acagcatgaagtgcaagaacgtggtgcccctctatgacctgctgctggagatgctggacgcccaccgcctacatgcgcccactagccgtggagg
ggcatccgtggaggagacggaccaaagccacttggccactgcgggctctacttcatcgcattccttgcaaaagtattacatcacggggg
gggggaggcagagggtttccctgccacggtctga
11. SEQ ID NO: 11-ERα amino acids 7-48
TKASGMALLHQIQGNELEPLNRPQLKIPLERPLGEVYLDSSK
12. SEQ ID NO: 12-ERα amino acids 13-54
ALLHQIQGNELEPLNRPQLKIPLERPLGEVYLDSSKPAVYNY
13. SEQ ID NO: 13-ERα amino acids 7-54
TKASGMALLHQIQGNELEPLNRPQLKIPLERPLGEVYLDSSKPAVYNY
14. SEQ ID NO: 14-ERα amino acids 1-60
MTMTLHTKASGMALLHQIQGNELEPLNRPQLKIPLERPLGEVYLDSSKPAVYNYPEGAAY
15. SEQ ID NO: 15-Mus musculus AF1 peptide
TKASGMALLHEPLNRPQLKMPMERDNSKPTVFNY
16. SEQ ID NO: 16-Rattus norvegicus AFI peptide
TKASGMALLHEPLNRPQLKMPMERDNSKPAVFNY
17. SEQ ID NO: 17-Deleted ERα amino acids
QIQGENL
18. SEQ ID NO: 18-Deleted ERα amino acids
PLGEVYL

1. A synthetic peptide comprising: a peptide from an activation function 1 (AF1) domain of an estrogen receptor alpha (ERα) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids.

2. The synthetic peptide of claim 1, wherein the at least 30 amino acids from the AF1 domain of the ERα protein are selected from the first 60 amino acids of the ERα protein.

3. The synthetic peptide of claim 1, wherein the synthetic peptide comprises 90% sequence identity to SEQ ID NO: 1.

4. (canceled)

5. (canceled)

6. The synthetic peptide of claim 1, wherein the AF1 domain comprises SEQ ID NO: 2, or a variant or fragment thereof.

7. The synthetic peptide of claim 1, wherein the cell penetrating peptide comprises SEQ ID NO: 3, or a variant or fragment thereof.

8. The synthetic peptide of claim 1, wherein the cell penetrating peptide comprises a transactivator of transcription (TAT) peptide.

9. The synthetic peptide of claim 1, wherein the synthetic peptide is further linked to a light emitting molecule.

10. The synthetic peptide of claim 1, wherein the light emitting molecule comprises a fluorescent molecule.

11. The synthetic peptide of claim 1, wherein the light emitting molecule comprises a fluorescein molecule, or a derivative thereof.

12. A nucleic acid encoding the synthetic peptide of claim 1.

13. A pharmaceutical composition for enhancing insulin sensitivity comprising a synthetic peptide comprising:
the synthetic peptide of claim 1; and
a pharmaceutically acceptable carrier selected from an excipient, a diluent, a salt, a buffer, a stabilizer, a solubilizer, a lipid, an emulsion, and nanoparticle.

14. A method of treating or preventing diabetes in a subject in need thereof, the method comprising administering a therapeutically effective amount of a synthetic peptide comprising: a peptide from an activation function 1 (AF1) domain of an estrogen receptor alpha (ERα) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids.

15. The method of claim 14, wherein the synthetic peptide comprises 90% sequence identity to SEQ ID NO: 1.

16. (canceled)

17. (canceled)

18. The method of claim 14, wherein the synthetic peptide interacts with an insulin receptor substrate (IRS) to activate insulin signaling in the subject.

19. The method of claim 18, wherein the IRS comprises an insulin receptor substrate 1 (IRS1).

20. The method of claim 14, wherein the synthetic peptide enhances insulin sensitivity in the subject.

21. (canceled)

22. (canceled)

23. (canceled)

24. The method of claim 14, wherein the cell penetrating peptide directs the synthetic peptide into a cell.

25. The method of claim 24, wherein the cell is a liver cell, a muscle cell, or an adipose cell.

26. (canceled)

27. (canceled)

28. (canceled)

29. The method of claim 14, wherein the type of diabetes is Type II diabetes.

30. A method of enhancing insulin sensitivity in a subject, the method comprising administering a therapeutically effective amount of a synthetic peptide comprising: a peptide from an activation function 1 (AF1) domain of an estrogen receptor alpha (ERα) protein and a cell penetrating peptide; wherein the cell penetrating peptide is linked to the amino terminus of the peptide from the AF1 domain of the ERα protein, and wherein the peptide from the AF1 domain of the ERα protein comprises at least 30 amino acids.

31.-46. (canceled)


SEQUENCE LISTING (18 sequences)

SEQ ID NO: 1 - protein, 45 aa, synthetic construct
  REGION 1..45: note = Description of sequence: ER synthetic peptide amino acid sequence
YGRKKRRQRRRTKASGMALLHEPLNRPQLKIPLERDSSKPAVYNY

SEQ ID NO: 2 - protein, 34 aa, synthetic construct
  REGION 1..34: note = Description of sequence: AF1 domain portion sequence
TKASGMALLHEPLNRPQLKIPLERDSSKPAVYNY

SEQ ID NO: 3 - protein, 11 aa, synthetic construct
  REGION 1..11: note = Description of sequence: Cell penetrating peptide amino acid sequence
YGRKKRRQRRR

SEQ ID NO: 4 - DNA, 135 bp, synthetic construct
  misc_feature 1..135: note = Description of sequence: ER synthetic peptide nucleic acid sequence
  source 1..135: mol_type = other DNA
tatggccgcaaaaaacgccgccagcgccgccgcaccaaagcatctgggatggccctactgcatgagcccctgaaccgtccgcagctcaagatccccctggagcgggacagcagcaagcccgccgtgtacaactac

SEQ ID NO: 5 - DNA, 102 bp, synthetic construct
  misc_feature 1..102: note = Description of sequence: AF1 domain portion nucleic acid sequence
  source 1..102: mol_type = other DNA
accaaagcatctgggatggccctactgcatgagcccctgaaccgtccgcagctcaagatccccctggagcgggacagcagcaagcccgccgtgtacaactac

SEQ ID NO: 6 - DNA, 33 bp, synthetic construct
  misc_feature 1..33: note = Description of sequence: Cell penetrating peptide nucleic acid sequence
  source 1..33: mol_type = other DNA
tatggccgcaaaaaacgccgccagcgccgccgc

SEQ ID NO: 7 - protein, 180 aa, synthetic construct
  REGION 1..180: note = Description of sequence: AF1 domain amino acid sequence
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAK

SEQ ID NO: 8 - DNA, 540 bp, synthetic construct
  misc_feature 1..540: note = Description of sequence: AF1 domain nucleic acid sequence
  source 1..540: mol_type = other DNA
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaag

SEQ ID NO: 9 - protein, 597 aa, Homo sapiens
  REGION 1..597: note = Description of sequence: FULL PROTEIN SEQUENCE FOR THE ER ALPHA PROTEIN
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGNRHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 10 - DNA, 1794 bp, Homo sapiens
  misc_feature 1..1794: note = Description of sequence: FULL NUCLEIC ACID SEQUENCE FOR THE ER ALPHA PROTEIN
  source 1..1794: mol_type = genomic DNA
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggtaatagacataacgactatatgtgtccagccaccaaccagtgcaccattgataaaaacaggaggaagagctgccaggcctgccggctccgtaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagcccccgatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 11 - protein, 42 aa, synthetic construct
  REGION 1..42: note = Description of sequence: ER-alpha amino acids 7-48
TKASGMALLHQIQGNELEPLNRPQLKIPLERPLGEVYLDSSK

SEQ ID NO: 12 - protein, 42 aa, synthetic construct
  REGION 1..42: note = Description of sequence: ER-alpha amino acids 13-54
ALLHQIQGNELEPLNRPQLKIPLERPLGEVYLDSSKPAVYNY

SEQ ID NO: 13 - protein, 48 aa, synthetic construct
  REGION 1..48: note = Description of sequence: ER-alpha amino acids 7-54
TKASGMALLHQIQGNELEPLNRPQLKIPLERPLGEVYLDSSKPAVYNY

SEQ ID NO: 14 - protein, 60 aa, synthetic construct
  REGION 1..60: note = Description of sequence: ER-alpha amino acids 1-60
MTMTLHTKASGMALLHQIQGNELEPLNRPQLKIPLERPLGEVYLDSSKPAVYNYPEGAAY

SEQ ID NO: 15 - protein, 34 aa, synthetic construct
  REGION 1..34: note = Description of sequence: AF1 peptide
TKASGMALLHEPLNRPQLKMPMERDNSKPTVFNY

SEQ ID NO: 16 - protein, 34 aa, Rattus norvegicus
  REGION 1..34: note = Description of sequence: AFI peptide
TKASGMALLHEPLNRPQLKMPMERDNSKPAVFNY

SEQ ID NO: 17 - protein, 7 aa, synthetic construct
  REGION 1..7: note = Description of sequence: Deleted ER-alpha amino acids
QIQGENL

SEQ ID NO: 18 - protein, 7 aa, synthetic construct
  REGION 1..7: note = Description of sequence: Deleted ER-alpha amino acids
PLGEVYL